SEQUENCE LISTING (92 sequences)

SEQ ID NO: 1 - DNA, 98 bp, Artificial Sequence
  Synthetic construct
tggcaggatatattgtggtgtaaacaaattgacgcttagacaacttaataacacattgcggacgtttttaatgtactgattaatggcgcgccctcgag

SEQ ID NO: 2 - DNA, 5 bp, Artificial Sequence
  Synthetic construct
agagg

SEQ ID NO: 3 - DNA, 51 bp, Artificial Sequence
  Synthetic construct
acacgctgaaatcaccagtctctctctacaaatctatctctggcgccaaaa

SEQ ID NO: 4 - DNA, 157 bp, Artificial Sequence
  Synthetic construct
tggcaggatatattgtggtgtaaacaaattgacgcttagacaacttaataacacattgcggacgtttttaatgtactgattaatggcgcgccctcgagagaggacacgctgaaatcaccagtctctctctacaaatctatctctggcgccaaaaatg

SEQ ID NO: 5 - DNA, 51 bp, Artificial Sequence
  Synthetic construct
acacgctgaaatcaccagtctctctctacaaatctatctctgagctcaaaa

SEQ ID NO: 6 - DNA, 157 bp, Artificial Sequence
  Synthetic construct
tggcaggatatattgtggtgtaaacaaattgacgcttagacaacttaataacacattgcggacgtttttaatgtactgattaatggcgcgccctcgagagaggacacgctgaaatcaccagtctctctctacaaatctatctctgagctcaaaaatg

SEQ ID NO: 7 - DNA, 56 bp, Artificial Sequence
  Synthetic construct
agaggacacgctgaaatcaccagtctctctctacaaatctatctctgagctcaaca

SEQ ID NO: 8 - DNA, 157 bp, Artificial Sequence
  Synthetic construct
tggcaggatatattgtggtgtaaacaaattgacgcttagacaacttaataacacattgcggacgtttttaatgtactgattaatggcgcgccgtcgacagaggacacgctgaaatcaccagtctctctctacaaatctatctctgagctcaacaatg

SEQ ID NO: 9 - DNA, 32 bp, Artificial Sequence
  Synthetic construct
tggcaggatatattgtggtgtaaacaaattga

SEQ ID NO: 10 - DNA, 6 bp, Artificial Sequence
  Synthetic construct
gagagg

SEQ ID NO: 11 - DNA, 92 bp, Artificial Sequence
  Synthetic construct
tggcaggatatattgtggtgtaaacaaattgagagaggacacgctgaaatcaccagtctctctctacaaatctatctctggcgccaaaaatg

SEQ ID NO: 12 - DNA, 27 bp, Artificial Sequence
  Synthetic construct
tggcaggatatattgtggtgtaaacga

SEQ ID NO: 13 - DNA, 87 bp, Artificial Sequence
  Synthetic construct
tggcaggatatattgtggtgtaaacgagagaggacacgctgaaatcaccagtctctctctacaaatctatctctggcgccaaaaatg

SEQ ID NO: 14 - DNA, 25 bp, Artificial Sequence
  Synthetic construct
tggcaggatatattgtggtgtaaac

SEQ ID NO: 15 - DNA, 28 bp, Artificial Sequence
  Synthetic construct
tggcaggatatattgtggtgtaaacatg

SEQ ID NO: 16 - protein, 332 aa, Homo sapiens
AAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 17 - DNA, 1155 bp, Artificial Sequence
  Synthetic construct
atgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgtcgacatcggtaccccgtcttgatga

SEQ ID NO: 18 - protein, 857 aa, Leishmania major
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVVEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRR

SEQ ID NO: 19 - DNA, 2574 bp, Artificial Sequence
  Synthetic construct
atgggtaagcgtaagggcaacagccttggtgattctggttctgctgctaccgcttctagagaggcttctgctcaagctgaagatgctgcttctcagaccaagactgctagccctcctgctaaggttatcctgcttcctaagaccttgaccgacgagaaggactttatcgggatcttcccttttccgttctggcctgtgcatttcgtgcttactgttgtggctcttttcgtgctggctgcttcttgctttcaggctttcaccgtgaggatgatcagcgtgcagatctacggttacctgatccacgagttcgacccgtggtttaattacagggctgccgagtacatgtctacccatggttggtctgctttcttcagctggttcgactacatgagctggtatcctcttggtaggcctgtgggttctactacttatcctggacttcagcttaccgctgtggctattcatagagctttggctgctgctggcatgccgatgtctcttaacaatgtgtgcgtgctgatgcctgcatggttcggtgctattgctactgctactttggccttctgtacctacgaggcttcaggttctactgttgctgctgcagctgctgctctgagcttctctattattcctgctcacctgatgcggagcatggctggtgaatttgacaacgagtgcattgctgtggctgctatgcttctgactttctactgctgggtgagatcccttaggaccagatcttcttggcctattggtgtgcttaccggtgttgcttacggttacatggctgcagcttggggcggttacattttcgtgttgaacatggtggctatgcacgccggcattagctctatggttgattgggctcgtaatacttacaacccgagccttcttagggcttacacccttttctacgtggtgggaaccgctattgctgtttgtgttcctcctgtgggcatgagccctttcaagtctcttgaacagcttggtgctctgctggtgcttgttttcttgtgcggacttcaggtttgcgaggtgttgagagctagagctggtgttgaggttaggtccagggctaacttcaagatcagagtgagggtgttctccgttatggctggcgttgcagctcttgctatttctgtgcttgctcctaccggttacttcggtcctttgtctgttagggtgagagccttgttcgttgagcataccaggactggtaaccctctggttgattctgttgctgagcatcagcctgcttctccagaggctatgtgggcttttcttcatgtgtgcggtgtgacttggggtctgggttctattgtgttggctgtgtctaccttcgtgcactacagcccttctaaggtgttctggcttctgaactctggcgccgtgtactacttctctactaggatggctaggctcctgcttctttctggacctgctgcttgtcttagcaccggtattttcgtgggcaccattcttgaagctgccgtgcagttgtctttctgggattctgatgctaccaaggccaaaaagcagcaaaagcaggctcagaggcatcagagaggtgctggtaaaggttctggtagggatgacgctaagaatgctactaccgctcgggctttctgtgatgtgtttgctggttcttctctggcttggggtcaccgtatggtgctttctattgcaatgtgggctcttgtgactaccaccgccgtttctttcttctcctccgaattcgcttcccacagcactaagttcgctgagcagtcaagcaacccgatgattgtgttcgctgctgttgtgcagaatcgtgctactggcaagcctatgaacctgctggtggatgattacctgaaggcttacgagtggctgagggattctactcctgaggatgctagagttctcgcttggtgggattacggctaccagattaccggtattggcaacaggacctctctggctgatggtaatacttggaaccacgagcacattgccaccatcggtaagatgcttactagccctgttgtcgaggctcactctcttgttaggcacatggctgattacgtgctgatttgggctggtcagtctggcgatcttatgaagtctcctcacatggctaggatcggcaactctgtgtaccacgatatctgccctgatgatcctctttgccagcagttcggtttccaccggaatgattactctcggcctactcctatgatgcgggcttctcttctttacaaccttcacgaggctggtaagcggaaaggtgttaaggtgaacccgagcttgttccaagaggtgtacagctctaagtacggcctggtgaggatcttcaaggtgatgaatgtgagcgccgagagcaagaagtgggttgcagatcctgctaatagggtgtgccatcctcctggttcttggatttgtcctggtcagtaccctccggccaaagaaattcaagagatgctggctcatagggtgccgttcgatcaggttaccaacgctgatcggaagaacaacgtggggtcttatcaagaggagtacatgcggaggatgcgtgagtctgagaatagaaggtaa

SEQ ID NO: 20 - protein, 586 aa, Artificial Sequence
  Synthetic construct
MRSASNSNAPNKQWRNWLPLFVALVIIAEFSFLVRLDVAEVRDNDHPDHSSRELSKILAKLERLKQQNEDLRRMAESLRIPEGPIDQGPAIGRVRVLEEQLVKAKEQIENYKKQTRNGLGKDHEILRRRIENGAKELWFFLQSELKKLKNLEGNELQRHADEFLLDLGHHERSIMTDLYYLSQTDGAGDWREKEAKDLTELVQRRITYLQNPKDCSKAKKLVCNINKGCGYGCQLHHVVYCFMIAYGTQRTLILESQNWRYATGGWETVFRPVSETCTDRSGISTGHWSGEVKDKNVQVVELPIVDSLHPRPPYLPLAVPEDLADRLVRVHGDPAVWWVSQFVKYLIRPQPWLEKEIEEATKKLGFKHPVIGVHVRRTDKVGTEAAFHPIEEYMVHVEEHFQLLARRMQVDKKRVYLATDDPSLLKEAKTKYPNYEFISDNSISWSAGLHNRYTENSLRGVILDIHFLSQADFLVCTFSSQVCRVAYEIMQTLHPDASANFHSLDDIYYFGGQNAHNQIAIYAHQPRTADEIPMEPGDIIGVAGNHWDGYSKGVNRKLGRTGLYPSYKVREKIETVKYPTYPEAEK

SEQ ID NO: 21 - DNA, 1761 bp, Artificial Sequence
  Synthetic construct
atgaggtctgcttctaattctaacgctccaaacaagcaatggaggaactggcttccacttttcgtggctcttgtgatcatcgctgaattctctttcttggttagattggacgttgcagaggtgagggacaacgaccacccagatcactcatctagggagttgtctaaaatccttgctaaattggaaaggttgaaacaacaaaatgaggacttgaggaggatggctgagtctttgagaatcccagagggacctatcgaccaaggaccagcaatcggtagggtgagagtgttggaggagcagcttgttaaggcaaaggagcaaatcgaaaactacaagaagcagactaggaacggattgggaaaggaccacgaaatccttaggaggagaatcgagaacggagctaaggaactttggtttttccttcaatcagagttgaagaagttgaagaatttggaaggtaacgagttgcagagacacgctgacgagttccttcttgatttgggtcaccacgagaggtcaatcatgactgacttgtactatttgtctcagactgacggtgctggagactggagagagaaggaggctaaggacttgactgagcttgtgcagaggagaattacatatcttcaaaacccaaaagattgttcaaaagcaaagaagttggtgtgcaatatcaacaagggatgcggatacggatgtcagttgcaccacgttgtgtactgcttcatgattgcttacggaactcagaggactttgattcttgaatctcaaaactggaggtacgctacaggtggatgggaaacagtgttcaggccagtgtctgagacatgcacagacaggtctggtatctcaacaggtcactggtctggagaggtgaaggacaagaacgtgcaggtggttgagttgcctatcgttgactcattgcacccaaggccaccttacttgccacttgcagttcctgaggacttggctgacaggcttgttagggtgcatggagatcctgcagtgtggtgggtgtcacagtttgtgaagtaccttatcagaccacagccatggttggagaaagagatcgaggaggcaactaagaagcttggtttcaaacatccagtgatcggagtgcacgtgaggaggactgacaaggtgggaactgaagcagcattccaccctattgaggagtacatggtgcacgtggaggagcactttcagttgcttgcaaggaggatgcaggtggacaaaaagagggtgtaccttgctacagatgacccatctcttcttaaagaggctaagactaaataccctaattatgagttcatctcagacaactctatttcatggtcagctggattgcataatagatatactgaaaactcacttaggggagttattttggatattcatttcctttctcaggctgatttcttggtttgtactttctcttcacaagtttgtagagtggcttacgagatcatgcagacacttcacccagatgcttctgctaatttccactctttggacgatatttattatttcggtggtcaaaatgcacataaccaaattgcaatttacgctcatcagccaaggactgctgacgagattccaatggagcctggagacatcatcggtgtggcaggaaaccactgggatggttactcaaagggagtgaacaggaaattgggtagaactggtctttatccttcttacaaggtgagggaaaagatcgagacagtgaaataccctacatacccagaggcagagaagtga

SEQ ID NO: 22 - DNA, 148 bp, Artificial Sequence
  Synthetic construct
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactg

SEQ ID NO: 23 - DNA, 25 bp, Artificial Sequence
  Synthetic construct
tggcaggatatattgtggtgtaaac

SEQ ID NO: 24 - DNA, 162 bp, Artificial Sequence
  Synthetic construct
agattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 25 - DNA, 25 bp, Artificial Sequence
  Synthetic construct
tgacaggatatattggcgggtaaac

SEQ ID NO: 26 - DNA, 51 bp, Artificial Sequence; identical to SEQ ID NO: 3
  Synthetic construct

SEQ ID NO: 27 - DNA, 325 bp, Cauliflower mosaic virus
aacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtg

SEQ ID NO: 28 - DNA, 92 bp, Cauliflower mosaic virus
atatctccactgacgtaagggatgacgcacaatcccactatccttcgcaagacccttcctctatataaggaagttcatttcatttggagagg

SEQ ID NO: 29 - DNA, 519 bp, Artificial Sequence
  Synthetic construct
atggaaagggctattcagggaaatgatgctagagagcaggctaattctgaaagatgggatggtggatctggtggaactacttctccattcaagcttccagatgagtctccatcttggactgagtggaggcttcataacgatgagactaactccaatcaggataacccactcggattcaaagaatcttggggattcggaaaggttgtgttcaagcgttaccttaggtatgataggactgaggcttcacttcatagggttctcggatcttggactggtgattctgttaactacgctgcttctcgtttttttggattcgatcagatcggatgcacttactctattaggttcaggggagtgtctattactgtttctggtggatctaggactcttcaacacctttgcgagatggctattaggtctaagcaagagcttcttcagcttgctccaattgaggttgagtctaacgtttcaagaggatgtccagaaggtactgagactttcgagaaagaatccgagtga

SEQ ID NO: 30 - DNA, 53 bp, Artificial Sequence
  Synthetic construct
aaattgacgcttagacaacttaataacacattgcggacgtttttaatgtactg

SEQ ID NO: 31 - DNA, 7 bp, Artificial Sequence
  Synthetic construct
aaattga

SEQ ID NO: 32 - DNA, 30 bp, Artificial Sequence
  Synthetic construct
attaatggcgcgccctcgaggccccgtacg

SEQ ID NO: 33 - DNA, 20 bp, Artificial Sequence
  Synthetic construct
attaatggcgcgccctcgag

SEQ ID NO: 34 - DNA, 2 bp, Artificial Sequence
  Synthetic construct
ga

SEQ ID NO: 35 - DNA, 24 bp, Artificial Sequence
  Synthetic construct
attaatggcgcgccctcgaggccc

SEQ ID NO: 36 - DNA, 325 bp, Artificial Sequence; identical to SEQ ID NO: 27
  Synthtic construct

SEQ ID NO: 37 - DNA, 92 bp, Artificial Sequence; identical to SEQ ID NO: 28
  Synthetic construct

SEQ ID NO: 38 - DNA, 6 bp, Artificial Sequence
  Synthetic construct
gagagg

SEQ ID NO: 39 - DNA, 51 bp, Artificial Sequence; identical to SEQ ID NO: 3
  Synthetic construct

SEQ ID NO: 40 - protein, 21 aa, Artificial Sequence
  Synthetic construct
MAKTNLFLFLIFSLLLSLSSA

SEQ ID NO: 41 - protein, 28 aa, Homo sapiens
MHSKVTIICIRFLFWFLLLCMLIGKSHT

SEQ ID NO: 42 - DNA, 2665 bp, Artificial Sequence
  Synthetic construct
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgattaatggcgcgccctcgaggccccgtacgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctggcgccaaaaatgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgtcgacatcggtaccccgtcttgatgaagatcttccggatcgataatgaaatgtaagagatatcatatataaataataaattgtcgtttcatatttgcaatcttttttttacaaacctttaattaattgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttgctagcggccggccttaattaaagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 43 - DNA, 904 bp, Artificial Sequence
  Synthetic construct
tggcaggatatattgtggtgtaaacaaattgacgcttagacaacttaataacacattgcggacgtttttaatgtactgattaatggcgcgccctcgaggccccgtacgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctggcgccaaaaatg

SEQ ID NO: 44 - DNA, 2009 bp, Artificial Sequence
  Synthetic construct
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgattaatggcgcgccctcgagtgtgatatctccactgacgtaagggatgacgcacaatcccactatccttcgcaagacccttcctctatataaggaagttcatttcatttggagaggacacgctgaaatcaccagtctctctctacaaatctatctctggcgccaaaaatgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgtcgacatcggtaccccgtcttgatgaagatcttccggatcgataatgaaatgtaagagatatcatatataaataataaattgtcgtttcatatttgcaatcttttttttacaaacctttaattaattgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttgctagcggccggccttaattaaagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 45 - DNA, 248 bp, Artificial Sequence
  Synthetic construct
tggcaggatatattgtggtgtaaacaaattgacgcttagacaacttaataacacattgcggacgtttttaatgtactgattaatggcgcgccctcgagtgtgatatctccactgacgtaagggatgacgcacaatcccactatccttcgcaagacccttcctctatataaggaagttcatttcatttggagaggacacgctgaaatcaccagtctctctctacaaatctatctctggcgccaaaaatg

SEQ ID NO: 46 - DNA, 1918 bp, Artificial Sequence
  Synthetic construct
ctgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgattaatggcgcgccctcgagagaggacacgctgaaatcaccagtctctctctacaaatctatctctggcgccaaaaatgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgtcgacatcggtaccccgtcttgatgaagatcttccggatcgataatgaaatgtaagagatatcatatataaataataaattgtcgtttcatatttgcaatcttttttttacaaacctttaattaattgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttgctagcggccggccttaattaaagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 47 - DNA, 157 bp, Artificial Sequence; identical to SEQ ID NO: 4
  Synthetic construct

SEQ ID NO: 48 - DNA, 1853 bp, Artificial Sequence
  Synthetic construct
ctgatgggctgcctgtatcgagtggtgattttgtgccgagctgccggtcggggagctgttggctggctggtggcaggatatattgtggtgtaaacaaattgagagaggacacgctgaaatcaccagtctctctctacaaatctatctctggcgccaaaaatgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgtcgacatcggtaccccgtcttgatgaagatcttccggatcgataatgaaatgtaagagatatcatatataaataataaattgtcgtttcatatttgcaatcttttttttacaaacctttaattaattgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttgctagcggccggccttaattaaagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 49 - DNA, 92 bp, Artificial Sequence; identical to SEQ ID NO: 11
  Synthetic construct

SEQ ID NO: 50 - DNA, 1848 bp, Artificial Sequence
  Synthetic construct
ctgatgggctgcctgtatcgagtggtgattttgtgccgagctgccggtcggggagctgttggctggctggtggcaggatatattgtggtgtaaacgagagaggacacgctgaaatcaccagtctctctctacaaatctatctctggcgccaaaaatgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgtcgacatcggtaccccgtcttgatgaagatcttccggatcgataatgaaatgtaagagatatcatatataaataataaattgtcgtttcatatttgcaatcttttttttacaaacctttaattaattgtatgtatgacattttcttcttgttatattagggggaaataatgttaaataaaagtacaaaataaactacagtacatcgtactgaataaattacctagccaaaaagtacacctttccatatacttcctacatgaaggcattttcaacattttcaaataaggaatgctacaaccgcataataacatccacaaatttttttataaaataacatgtcagacagtgattgaaagattttattatagtttcgttatcttgctagcggccggccttaattaaagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagg

SEQ ID NO: 51 - DNA, 87 bp, Artificial Sequence; identical to SEQ ID NO: 13
  Synthetic construct

SEQ ID NO: 52 - DNA, 1155 bp, Artificial Sequence
  Synthetic construct
atgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggttgccgcagccatcggtcagtcctctggtgaactccgtaccggtggtgctcgtcctccaccgccgctgggtgcatctagccagccgcgtccgggtggcgacagctctccggttgtggattctggcccaggtccagcttctaacctgacgtctgttccggttccacataccaccgcgctcagcctgccggcgtgcccggaagaatctccgctgctggtaggccctatgctcatcgaattcaacatgccggtagacctggaactcgttgcgaagcagaacccgaacgtaaagatgggtggtcgctacgcccctcgtgattgcgtttccccgcacaaggtggccatcatcattcctttccgtaaccgtcaagagcacctgaaatactggctgtactacctgcacccggttctgcagcgtcagcagctcgactacggtatctacgttatcaaccaggcgggtgacaccatctttaaccgcgctaaactgctgaacgtgggtttccaggaggcgctcaaggattacgactacacctgcttcgttttctctgacgttgacctgatcccgatgaatgatcacaacgcctaccgttgcttttctcaaccacgtcacatctctgttgcgatggacaaattcggtttctctctcccgtatgtacagtacttcggtggcgtgtctgccctctctaagcagcaattcctgacgatcaacggtttcccgaacaattactggggttggggtggtgaagacgatgatatcttcaaccgcctcgtattccgcggtatgtctatcagccgtccgaatgcggtcgtgggccgctgccgtatgatccgtcacagccgtgacaagaagaacgagccgaacccgcagcgctttgaccgtatcgcgcacaccaaagaaactatgctgtctgacggcctgaactctctcacgtaccaagttctcgacgtacagcgttacccgctgtatacccagatcaccgttgacatcggaaccccgtcttgatga

SEQ ID NO: 53 - protein, 383 aa, Artificial Sequence
  Synthetic construct
MIHTNLKKKFSLFILVFLLFAVICVWKKGSDYEALTLQAKEFQMPKSQEKVAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 54 - DNA, 153 bp, Artificial Sequence
  Synthetic construct
atgattcacacgaacctgaagaagaagttcagcctcttcatcctggttttcctgctcttcgcggtaatctgcgtttggaagaagggttctgactacgaagccctcaccctccaggcgaaggaattccagatgccgaagtctcaggagaaggtt

SEQ ID NO: 55 - protein, 51 aa, Artificial Sequence
  Synthetic construct
MIHTNLKKKFSLFILVFLLFAVICVWKKGSDYEALTLQAKEFQMPKSQEKV

SEQ ID NO: 56 - DNA, 20 bp, Artificial Sequence
  Synthetic construct
attaatggcgcgccctcgag

SEQ ID NO: 57 - DNA, 98 bp, Artificial Sequence
  Synthetic construct
tggcaggatatattgtggtgtaaacaaattgacgcttagacaacttaataacacattgcggacgtttttaatgtactgattaatggcgcgccgtcgac

SEQ ID NO: 58 - DNA, 20 bp, Artificial Sequence
  Synthetic construct
attaatggcgcgccgtcgac

SEQ ID NO: 59 - DNA, 41 bp, Cauliflower mosaic virus
acacgctgaaatcaccagtctctctctacaaatctatctct

SEQ ID NO: 60 - DNA, 620 bp, Arabidopsis thaliana
attgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaaaa

SEQ ID NO: 61 - DNA, 178 bp, Arabidopsis thaliana
attgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaagctcataaaaa

SEQ ID NO: 62 - DNA, 623 bp, Arabidopsis thaliana
agaattgcctcgtcgtcttcagcttcatcggccgttgcatttcccggcgataagagagagaaagaggagaaagagtgagccagatcttcatcgtcgtggttcttgtttcttcctcgatctctcgatcttctgcttttgcttttccgattaaggtaattaaaacctccgatctacttgttcttgtgttggatctcgattacgatttctaagttaccttcaaaagttgtttccgatttgattttgattggaatttagatcggtcaaactattggaaatttttgatcctggcaccgattagctcaacgattcatgtttgacttgatcttgcgttgtatttgaaatcgatccggatcctttcgcttcttctgtcaataggaatctgaaatttgaaatgttagttgaagtttgacttcagattctgttgatttattgactgtaacattttgtcttccgatgagtatggattcgttgaaatctgctttcattatgattctattgatagatacatcatacattgaattgaatctactcatgaatgaaaagcctggtttgattaagaaagtgttttcggttttctcgatcaagattcagatctttatgtttttgattgcagatcgtagacc

SEQ ID NO: 63 - DNA, 162 bp, Arabidopsis thaliana
agaattgcctcgtcgtcttcagcttcatcggccgttgcatttcccggcgataagagagagaaagaggagaaagagtgagccagatcttcatcgtcgtggttcttgtttcttcctcgatctctcgatcttctgcttttgcttttccgattaagatcgtagacc

SEQ ID NO: 64 - protein, 477 aa, Artificial Sequence
  Synthetic construct
MAKTNLFLFLIFSLLLSLSSAQVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 236 aa, Artificial Sequence
  Synthetic construct
MAKTNLFLFLIFSLLLSLSSAEIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLRSPVTKSFNRGEC

SEQ ID NO: 66 - DNA, 1434 bp, Artificial Sequence
  Synthetic construct
atggctaaaactaatctgttcctttttcttattttctctttactcttgtccctcagttctgctcaggttcagttagttcaatctggcgcagaggtaaagaaacctggagctagtgtgaaagttagttgccaagctagcggatacaggttctctaattttgttatccactgggtccgtcaggctcctggacagagattcgaatggatggggtggattaatccttacaatggaaacaaggagtttagcgcaaaatttcaagatagagttactttcaccgccgatacaagcgctaatacagcctatatggaattgagatcattacgatctgctgacactgcagtctattactgcgccagggtcggcccatactcctgggatgactctcctcaagataattattacatggacgtgtggggtaagggtacaaccgtcatagtttcatctgcatccactaagggtcctagtgtttttcctctggcaccatcttcaaagtctacatctggcgggacagctgcacttggatgccttgtgaaggattattttcctgaaccagtaacagttagctggaactccggtgctttgacttcaggcgttcatacttttcctgcagtacttcagagtagtggattgtatagcttgtctagcgtcgttactgtgccttcctcttcccttgggacacaaacatacatttgcaatgttaaccataaaccatctaatactaaggttgacaagaaagccgagcctaaatcttgtgataagactcatacttgtcctccatgtcctgcccctgagttgctgggaggtccatccgtatttctcttccctccaaagccaaaggatactttgatgattagtcggacacctgaagtgacctgtgtcgtggtagacgtttcacatgaagatccagaagttaaatttaattggtacgtggatggagttgaggtgcataacgctaaaactaagcctagggaagagcaatataattcaacctacagagttgtgtcagtcttaacagtgcttcaccaagattggttaaacggtaaggaatataagtgcaaagtttcaaataaggctcttcctgctccaatagaaaagaccatttctaaagctaagggacaacctcgagaacctcaggtatataccctccctccaagtcgtgacgaattgacaaaaaaccaggtttctttgacctgtttggttaaaggtttttatcctagtgatatcgctgtggagtgggagtctaatggtcagcctgagaataactataagactactcctccagtcctcgatagcgatggttcattctttctttactctaaattgactgtagataaaagcagatggcaacaggggaacgtgttctcatgttcagttatgcacgaggcactgcacaatcattatactcaaaagtctctgtcattgagtcctggttga

SEQ ID NO: 67 - DNA, 711 bp, Artificial Sequence
  Synthetic construct
atggctaagactaacttgtttctctttttgatcttctcattgcttctctccttaagctctgctgaaatagttcttacacaatcaccaggaactcttagtttaagtcctggcgagcgggctaccttttcttgccgaagttcccactctatcagatcaagacgagttgcatggtatcaacacaagccaggacaagctccaagattagtgattcatggtgtaagcaatagggcttctgggatatctgatcgtttctcaggctcaggttcaggtacagactttacattgaccattaccagggttgagccagaggatttcgctctttactattgtcaggtttatggcgcaagttcttacacttttgggcagggaaccaaactggaaaggaaaaggactgtggctgcaccttctgtgttcatttttcctccatccgatgaacaactgaagtccggtactgccagtgttgtctgtctcttgaataacttttacccaagagaggctaaggttcagtggaaagttgataacgcccttcaatctggaaatagccaagaaagtgtaacagagcaggactctaaggattccacatattctctttcttcaacacttacactgagcaaagcagattacgaaaaacataaggtctatgcatgcgaagtcacacatcagggacttagatctcctgtgactaagagcttcaatcgtggtgagtgttga

SEQ ID NO: 68 - protein, 475 aa, Artificial Sequence
  Synthetic construct
MAKTNLFLFLIFSLLLSLSSAQVQLVQSGSGVKKPGASVRVSCWTSEDIFERTELIHWVRQAPGQGLEWIGWVKTVTGAVNFGSPDFRQRVSLTRDRDLFTAHMDIRGLTQGDTATYFCARQKFYTGGQGWYFDLWGRGTLIVVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 229 aa, Artificial Sequence
  Synthetic construct
MAKTNLFLFLIFSLLLSLSSAEIVLTQSPGTLSLSPGETASLSCTAASYGHMTWYQKKPGQPPKLLIFATSKRASGIPDRFSGSQFGKQYTLTITRMEPEDFARYYCQQLEFFGQGTRLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 1428 bp, Artificial Sequence
  Synthetic construct
atggctaaaacaaatctctttttattcttgattttctcccttttactttccttatcaagcgctcaagtgcaactcgttcagtctgggtctggagttaagaaacctggcgccagtgtgagggtttcatgttggacttccgaggacatttttgaacgtactgaacttattcactgggttagacaagctccaggtcaagggttggagtggattggctgggtcaagacagtaactggagctgtcaattttggatctccagatttcagacaacgagtgagcttgacacgggatagagatctttttacagcacatatggatataagaggtttgacacagggagacaccgctacatacttttgcgcaaggcagaaattctatactggaggtcagggctggtatttcgatttatggggtaggggaaccctgatcgtagtatcaagtgctagtactaagggaccaagcgtttttcctttagccccaagttctaagtccactagtggaggtaccgcagctcttggttgtttagtcaaagattatttcccagagccagttaccgtgagttggaacagtggtgctttgactagtggagtccatacattcccagctgttttgcaatctagtggattgtattcactctctagtgtggttaccgtgccatcatcaagtttaggaacacaaacatatatatgcaatgtgaatcataaaccaagcaacactaaagttgataagaaagtggaaccaaagtcatgcgacaaaacacatacttgtcctccatgccctgcacctgaattattgggaggtcctagtgtttttttatttccacctaaaccaaaagatacccttatgatttctaggacaccagaagttacttgtgtcgtggtcgatgtgtcccatgaagatccagaagttaaattcaattggtatgtggatggtgttgaagtgcataacgctaagactaagcctagggaggaacaatataattcaacttatagagtcgttagtgtccttactgtcctccaccaagattggttgaatggaaaggagtataaatgcaaagtctcaaataaggctctcccagcacctatcgaaaaaaccatatccaaggccaaaggacaacctagagagcctcaagtttatacacttcctccatctagggaagaaatgacaaagaaccaagtgagccttacatgtctcgttaagggtttctatcctagtgacattgccgttgaatgggagagtaatggacaacctgagaacaattataagactacacctccagtcttggatagtgatggttctttctttttgtattctaaattaactgttgacaaatcaagatggcaacagggaaatgttttttcatgttctgtcatgcacgaggctcttcacaatcattatactcaaaaatcacttagccttagcccaggataa

SEQ ID NO: 71 - DNA, 690 bp, Artificial Sequence
  Synthetic construct
atggctaaaacaaatctctttttattcttgattttctcccttttactttccttatcaagcgctgagatagttttaacacaaagccctggcaccctttctctatctccaggtgaaactgcttcgctttcatgcactgctgccagttatggacatatgacatggtatcaaaagaaacctggacagccgccaaagttgcttatctttgcaaccagtaaacgtgcatctggtattcccgatcgattctccggttcacagttcggcaagcagtatactctcacgattactaggatggaacctgaagactttgctagatactactgtcaacagttggagtttttcgggcaaggaacaagactggagatcagaaggaccgtggctgcaccaagtgtgttcatatttcctccatccgatgaacaattgaagagtggtaccgcaagcgtcgtgtgtttattgaataacttttacccaagggaagccaaagttcaatggaaagttgataatgctctccaaagtggaaactcacaagaaagtgttacagagcaagactcaaaagattccacttatagcttatcatctacacttacactctcaaaagcagactatgaaaaacacaaagtctacgcttgcgaagtcactcatcaaggactttcttcaccagttacaaagagtttcaatagaggagagtgttaa

SEQ ID NO: 72 - protein, 482 aa, Artificial Sequence
  Synthetic construct
MAKTNLFLFLIFSLLLSLSSAQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 234 aa, Artificial Sequence
  Synthetic construct
MAKTNLFLFLIFSLLLSLSSASDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVLGQPKAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 1449 bp, Artificial Sequence
  Synthetic construct
atggctaaaacaaatctctttttattcttgattttctcccttttactttccttatcaagcgctcaaatgcagttgcaagaatctggtcctggacttgttaaacctagcgagactttgtcattaacatgctctgtctcaggtgccagtatttctgatagttactggtcatggatacggagaagtccaggtaaaggactcgagtggattgggtatgtgcacaagtctggtgatacaaattactcacctagtcttaagtccagagtcaatttgagccttgacacctccaagaatcaagtttctttgagcttagtggctgcaaccgctgcagattctggaaaatactattgtgctaggactctgcatgggcgacgtatctacggcattgttgcttttaacgaatggtttacttatttctatatggatgtttggggcaacggtactcaagtaacagtatcaagtgctagtactaagggaccaagcgtttttcctttagccccaagttctaagtccactagtggaggtaccgcagctcttggttgtttagtcaaagattatttcccagagccagttaccgtgagttggaacagtggtgctttgactagtggagtccatacattcccagctgttttgcaatctagtggattgtattcactctctagtgtggttaccgtgccatcatcaagtttaggaacacaaacatatatatgcaatgtgaatcataaaccaagcaacactaaagttgataagagagtggaaccaaagtcatgcgacaaaacacatacttgtcctccatgccctgcacctgaattattgggaggtcctagtgtttttttatttccacctaaaccaaaagatacccttatgatttctaggacaccagaagttacttgtgtcgtggtcgatgtgtcccatgaagatccagaagttaaattcaattggtatgtggatggtgttgaagtgcataacgctaagactaagcctagggaggaacaatataattcaacttatagagtcgttagtgtccttactgtcctccaccaagattggttgaatggaaaggagtataaatgcaaagtctcaaataaggctctcccagcacctatcgaaaaaaccatatccaaggccaaaggacaacctagagagcctcaagtttatacacttcctccatctagggaagaaatgacaaagaaccaagtgagccttacatgtctcgttaagggtttctatcctagtgacattgccgttgaatgggagagtaatggacaacctgagaacaattataagactacacctccagtcttggatagtgatggttctttctttttgtattctaaattaactgttgacaaatcaagatggcaacagggaaatgttttttcatgttctgtcatgcacgaggctcttcacaatcattatactcaaaaatcacttagccttagcccaggataa

SEQ ID NO: 75 - DNA, 705 bp, Artificial Sequence
  Synthetic construct
atggctaaaacaaatctctttttattcttgattttctcccttttactttccttatcaagcgcttctgacatatccgtcgcacctggagagacagctcgtatcagctgcggtgaaaaatcattagggagcagagccgttcaatggtatcaacatagggctggtcaggcaccatctttgatcatttacaacaatcaagatcggccatcaggtattcctgaacgattttctggttctcctgattcaccatttggaacaactgctaccctcactattacaagtgttgaagctggggacgaggctgattactattgtcacatatgggatagtagagtgccaaccaagtgggtattcggcggaggcactactcttactgttctgggacagccaaaggctgcaccaagtgtgttcatatttcctccatccgatgaacaattgaagagtggtaccgcaagcgtcgtgtgtttattgaataacttttacccaagggaagccaaagttcaatggaaagttgataatgctctccaaagtggaaactcacaagaaagtgttacagagcaagactcaaaagattccacttatagcttatcatctacacttacactctcaaaagcagactatgaaaaacacaaagtctacgcttgcgaagtcactcatcaaggactttcttcaccagttacaaagagtttcaatagaggagagtgttaa

SEQ ID NO: 76 - DNA, 78 bp, Artificial Sequence
  Synthetic construct
tggcaggatatattgtggtgtaaacaaattgacgcttagacaacttaataacacattgcggacgtttttaatgtactg

SEQ ID NO: 77 - DNA, 20 bp, Artificial Sequence
  Synthetic construct
attaatggcgcgccgtcgac

SEQ ID NO: 78 - DNA, 254 bp, Artificial Sequence
  Synthetic construct
gatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcg

SEQ ID NO: 79 - DNA, 552 bp, Artificial Sequence
  Synthetic construct
tcagatctcagtaactggaagaactggtcttggtggaactggaagtgagaaatcgagctgccagaatccaacatcatgccaatttccgtgcttaaaaccagcagctctaagcattcctcttggagcatatccaagagcctcatgcattctaacagatggatcgtttgggagtccaatcacagcaacaacagacttgaatccttgagcctcaagagacttgagaaggtgagtgtaaagagtagatccaagtccagtcctctgatgtcttggtgaaacgtaaacagtggactcagcagtccaatcataagcattcctagccttccatggtccagcataagcaattccagcaacttcaccatcaacttcagcaacaagccatggatacctttccctgagcctaacaagatcatcagtccattcttgtggctcttgtggttcagtcctaaagttcacagtggaagtctcaatgtagtggttcacaatagtgcacacagctggcatatcagcttcagtagcccttctaatatcagctggccttctttctggagacat

SEQ ID NO: 80 - DNA, 6 bp, Artificial Sequence
  Synthetic construct
ggatcc

SEQ ID NO: 81 - DNA, 207 bp, Artificial Sequence
  Synthetic construct
tgcagattatttggattgagagtgaatatgagactctaattggataccgaggggaatttatggaacgtcagtggagcatttttgacaagaaatatttgctagtgatagtgaccttaggcgacttttgaacgcgcaataatggtttctgacgtatgtgcttagctcattaaactccagaaacccgcggctcagtggctccttcaacgt

SEQ ID NO: 82 - DNA, 18 bp, Artificial Sequence
  Synthetic construct
gggcccggcgccgctagc

SEQ ID NO: 83 - DNA, 54 bp, Artificial Sequence
  Synthetic construct
tattcccttgttctacaggtgggcgcctgattaccaaaacttgcaacttgaaaa

SEQ ID NO: 84 - DNA, 6 bp, Artificial Sequence
  Synthetic construct
actagt

SEQ ID NO: 85 - DNA, 349 bp, Artificial Sequence
  Synthetic construct
aagataacgaaactataataaaatctttcaatcactgtctgacatgttattttataaaaaaatttgtggatgttattatgcggttgtagcattccttatttgaaaatgttgaaaatgccttcatgtaggaagtatatggaaaggtgtactttttggctaggtaatttattcagtacgatgtactgtagtttattttgtacttttatttaacattatttccccctaatataacaagaagaaaatgtcatacatacaattaattaaaggtttgtaaaaaaaagattgcaaatatgaaacgacaatttattatttatatatgatatctcttacatttcattatcgatccgga

SEQ ID NO: 86 - DNA, 6 bp, Artificial Sequence
  Synthetic construct
ctcgag

SEQ ID NO: 87 - DNA, 1002 bp, Artificial Sequence
  Synthetic construct
tcatcaagacggggttccgatgtcaacggtgatctgggtatacagcgggtaacgctgtacgtcgagaacttggtacgtgagagagttcaggccgtcagacagcatagtttctttggtgtgcgcgatacggtcaaagcgctgcgggttcggctcgttcttcttgtcacggctgtgacggatcatacggcagcggcccacgaccgcattcggacggctgatagacataccgcggaatacgaggcggttgaagatatcatcgtcttcaccaccccaaccccagtaattgttcgggaaaccgttgatcgtcaggaattgctgcttagagagggcagacacgccaccgaagtactgtacatacgggagagagaaaccgaatttgtccatcgcaacagagatgtgacgtggttgagaaaagcaacggtaggcgttgtgatcattcatcgggatcaggtcaacgtcagagaaaacgaagcaggtgtagtcgtaatccttgagcgcctcctggaaacccacgttcagcagtttagcgcggttaaagatggtgtcacccgcctggttgataacgtagataccgtagtcgagctgctgacgctgcagaaccgggtgcaggtagtacagccagtatttcaggtgctcttgacggttacggaaaggaatgatgatggccaccttgtgcggggaaacgcaatcacgaggggcgtagcgaccacccatctttacgttcgggttctgcttcgcaacgagttccaggtctaccggcatgttgaattcgatgagcatagggcctaccagcagcggagattcttccgggcacgccggcaggctgagcgcggtggtatgtggaaccggaacagacgtcaggttagaagctggacctgggccagaatccacaaccggagagctgtcgccacccggacgcggctggctagatgcacccagcggcggtggaggacgagcaccaccggtacggagttcaccagaggactgaccgatggctgcggc

SEQ ID NO: 88 - DNA, 153 bp, Artificial Sequence
  Synthetic construct
aaccttctcctgagacttcggcatctggaattccttcgcctggagggtgagggcttcgtagtcagaacccttcttccaaacgcagattaccgcgaagagcaggaaaaccaggatgaagaggctgaacttcttcttcaggttcgtgtgaatcat

SEQ ID NO: 89 - DNA, 6 bp, Artificial Sequence
  Synthetic construct
aagctt

SEQ ID NO: 90 - DNA, 25 bp, Artificial Sequence
  Synthetic construct
tgacaggatatattggcgggtaaac

SEQ ID NO: 91 - DNA, 129 bp, Artificial Sequence
  Synthetic construct
tgacaggatatattggcgggtaaacctaagagaaaagagcgtttattagaataatcggatatttaaaagggcgtgaaaaggtttatccgttcgtccatttgtatgtgcatgccaaccacagggttcccc

SEQ ID NO: 92 - DNA, 1858 bp, Artificial Sequence
  Synthetic construct
aagataacgaaactataataaaatctttcaatcactgtctgacatgttattttataaaaaaatttgtggatgttattatgcggttgtagcattccttatttgaaaatgttgaaaatgccttcatgtaggaagtatatggaaaggtgtactttttggctaggtaatttattcagtacgatgtactgtagtttattttgtacttttatttaacattatttccccctaatataacaagaagaaaatgtcatacatacaattaattaaaggtttgtaaaaaaaagattgcaaatatgaaacgacaatttattatttatatatgatatctcttacatttcattatcgatccggaggtacctcatcaccttctattctcagactcacgcatcctccgcatgtactcctcttgataagaccccacgttgttcttccgatcagcgttggtaacctgatcgaacggcaccctatgagccagcatctcttgaatttctttggccggagggtactgaccaggacaaatccaagaaccaggaggatggcacaccctattagcaggatctgcaacccacttcttgctctcggcgctcacattcatcaccttgaagatcctcaccaggccgtacttagagctgtacacctcttggaacaagctcgggttcaccttaacacctttccgcttaccagcctcgtgaaggttgtaaagaagagaagcccgcatcataggagtaggccgagagtaatcattccggtggaaaccgaactgctggcaaagaggatcatcagggcagatatcgtggtacacagagttgccgatcctagccatgtgaggagacttcataagatcgccagactgaccagcccaaatcagcacgtaatcagccatgtgcctaacaagagagtgagcctcgacaacagggctagtaagcatcttaccgatggtggcaatgtgctcgtggttccaagtattaccatcagccagagaggtcctgttgccaataccggtaatctggtagccgtaatcccaccaagcgagaactctagcatcctcaggagtagaatccctcagccactcgtaagccttcaggtaatcatccaccagcaggttcataggcttgccagtagcacgattctgcacaacagcagcgaacacaatcatcgggttgcttgactgctcagcgaacttagtgctgtgggaagcgaattcggaggagaagaaagaaacggcggtggtagtcacaagagcccacattgcaatagaaagcaccatacggtgaccccaagccagagaagaaccagcaaacacatcacagaaagcccgagcggtagtagcattcttagcgtcatccctaccagaacctttaccagcacctctctgatgcctctgagcctgcttttgctgctttttggccttggtagcatcagaatcccagaaagacaactgcacggcagcttcaagaatggtgcccacgaaaataccggtgctaagacaagcagcaggtccagaaagaagcaggagcctagccatcctagtagagaagtagtacacggcgccagagttcagaagccagaacaccttagaagggctgtagtgcacgaaggtagacacagccaacacaatagaacccagaccccaagtcacaccgcacacatgaagaaaagcccacatagcctctggagaagcaggctgatgctcagcaacagaatcaaccagagggttaccagtcctggtatgctcaacgaacaaggctctcaccctaacagacaaaggaccgaagtaaccggtaggagcaagcacagaaatagcaagagctgcaacgccagccataacggagaacaccctcactctgatcttgaagttagc

(19) United States
(12) Patent Application Publication
(10) Pub. No.: US 2022/0135992 A1
(43) Pub. Date: May 5, 2022

(54) T-DNA VECTORS WITH ENGINEERED 5' SEQUENCES UPSTREAM OF POST-TRANSLATIONAL MODIFICATION ENZYMES AND METHODS OF USE THEREOF

(71) Applicant: PlantForm Corporation, Toronto (CA)

(72) Inventors: Michael D. McLean, Guelph (CA); John D. Cossar, Guelph (CA); Wing-Fai Cheung, Scarborough (CA); Haifeng Wang, Puslinch (CA)

(21) Appl. No.: 17/435,946

(22) PCT Filed: Feb. 27, 2020
(86) PCT No.: PCT/CA2020/050260

§ 371 Date: Sep. 2, 2021

Related U.S. Application Data
(60) Provisional application No. 62/814,374, filed on Mar. 6, 2019.

(51) Int. Cl. C12N 15/82; C12N 9/12; C12P 21/00

(52) U.S. Cl. CPC C12N 15/8245; C12N 9/1241; C12P 21/005; C12N 15/8258; C12Y 207/07012

(57) ABSTRACT

Plant T-DNA expression vectors with engineered 5′ sequences for driving transcription of genes encoding post-translational modification enzymes are provided. Also methods of optimizing expression and glycosylation of recombinant protein produced in plants by utilizing plant T-DNA expression vectors with engineered 5′ sequences for driving transcription of genes encoding post-translational modification enzymes.

### CROSS-REFERENCE TO RELATED APPLICATIONS

This disclosure claims the benefit of U.S. provisional application No. 62/814,374 filed Mar. 6, 2019, the contents of which are incorporated herein by reference in their entirety.

### FIELD

The present disclosure relates to plant T-DNA expression vectors with engineered 5′ sequences for driving transcription of genes encoding proteins such as post-translational modification enzymes. The disclosure also relates to methods of controlling glycosylation of recombinant protein produced in plants by utilizing plant T-DNA expression vectors with engineered 5′ sequences for driving transcription of genes encoding post-translational modification enzymes.

### BACKGROUND

Production of valuable recombinant proteins in plants often involves more than just insertion of genes encoding these proteins (i.e., “target” proteins) into plants and allowing sufficient time for expression of the target proteins prior to their subsequent extraction and purification. Many target proteins, such as therapeutic antibodies, serum proteins and enzymes intended for replacement therapies are post-translationally modified by the addition of glycans, i.e., sugar moieties. These modifications are known to affect both the specific functional activities of these molecules as well as their residence times in the serum of treated patients (i.e., pharmacokinetics).

A plant-based production method for valuable recombinant proteins should therefore be capable of optimal post-translational glycosylation of target proteins. This will ensure that recombinant protein products have appropriate functional activities and pharmacokinetic properties.

Indeed, most therapeutic protein drugs, also known as biologics (MCLEAN AND HALL 2012), exist as mixtures of glycoproteins that are identical in amino acid sequence composition yet variable in the amounts of different glycan moieties which they possess due to activities of multiple post-translational modification enzymes. The complex nature of these glycoprotein mixtures creates tremendous challenges for pharmaceutical scientists developing novel production systems for the manufacture of biosimilar versions of these drugs, as innovator biologic drugs each possess their own characteristic amounts of various glycan species. It is inherently difficult to match glycan species compositions between production systems, and this difficulty increases if a novel production system is inherently different from an innovator drug production system. Such will be the case for biosimilar production systems using plant-based expression, as most biologic drugs are produced using mammalian CHO (Chinese hamster ovary), or SP2 and NSO (both murine) cell-based expression systems.

Reduced expression of transgenes encoding post-translational modification enzymes allows for greater control of post-translational modification activities, resulting in less complex mixtures of glycans with little to no incompletely processed glycans on plant produced recombinant target glycoproteins (KALLOLIMATH et al. 2017). Accordingly, a number of attempts have been made to reduce the complexity of glycans, the composition of these glycans, and the level of aglycosylation on recombinant target proteins using transient expression processes in plants.

However, complete glycosylation is still not achieved due in part to the fact that transient expression processes have an inherent difficulty overcoming such problems as simultaneous transient expression of target proteins and of post-translational modification enzymes. Thus, some target protein is produced before post-translational modification enzyme activities commence, resulting in populations of target proteins that have appreciable amounts of aglycosylated glycans or with incompletely matured glycans.

New plant expression vectors, systems and methods are therefore needed to generate stable transgenic host plants for the production of recombinant proteins with glycan profiles that are similar to those of innovator biologic drugs such as therapeutic antibodies, serum proteins and enzymes intended for replacement therapies.

### SUMMARY

The inventors have shown that T-DNA vectors with engineered 5′ sequences upstream of a post-translational modification enzyme coding sequence allow control of the transcriptional activity of the post-translational modification enzyme.

In particular, the present inventors have shown that plant expression vectors comprising a nucleic acid molecule encoding a post-translational modification enzyme, wherein the vector lacks a traditional promoter sequence for the nucleic acid molecule can be used for producing recombinant proteins in plants with optimized glycosylation patterns. The inventors have also shown that plant expression vectors comprising a nucleic acid molecule encoding a post-translational modification enzyme, wherein the vector lacks both a traditional promoter sequence and a 5′ untranslated region (5′UTR) sequence for the nucleic acid molecule can be used for producing recombinant proteins in plants with optimized glycosylation patterns.

Accordingly, the disclosure provides a plant T-DNA vector comprising a T-DNA region flanked by a Left Border sequence and a Right Border sequence, wherein the T-DNA region comprises a nucleic acid molecule encoding a protein of interest, optionally a post-translational modification (PTM) enzyme, and wherein the T-DNA region lacks a traditional promoter sequence for the nucleic acid molecule. In one embodiment, the T-DNA region lacks both a traditional promoter sequence and a 5′ untranslated region (5′UTR) sequence for the nucleic acid molecule.

The disclosure also provides a plant T-DNA vector comprising a T-DNA region flanked by a Left Border sequence and a Right Border sequence, wherein the T-DNA region comprises a nucleic acid molecule encoding a protein of interest, optionally a post-translational modification (PTM) enzyme, and wherein

(a) the ATG start of the translation codon of the nucleic acid sequence encoding the protein of interest is directly adjacent to the Left Border sequence or the Right Border sequence;

(b) the ATG start of the translation codon of the nucleic acid sequence encoding the protein of interest is within 10, 9, 8, 7, 6, 5 or fewer nucleotides of the Left Border sequence or the Right Border sequence;

(c) the ATG start of the translation codon of the nucleic acid sequence encoding the protein of interest is directly adjacent to a UTR sequence, and the UTR sequence is directly adjacent to the Left Border sequence or the Right Border sequence; or

(d) the ATG start of the translation codon of the nucleic acid sequence encoding the protein of interest is directly adjacent to a UTR sequence, and the UTR sequence is separated by an upstream sequence of 100 base pairs or less from the Left Border sequence or the Right Border sequence.

In one embodiment, the upstream sequence comprises a fragment of a promoter sequence. Optionally, the fragment consists of no more than 3, 4, 5, 6, 7, 8, 9, 10, 15 or 20 contiguous base pairs of the promoter sequence.

In another embodiment,

(a) the left border sequence comprises or consists of a sequence as set out in SEQ ID No:23, or a sequence having at least 50, 60, 70, 75, 80, 85, 90, 95 or 99% sequence identity to SEQ ID No: 23.

(b) the right border sequence comprises or consists of SEQ ID No: 25, or a sequence having at least 50, 60, 70, 75, 80, 85, 90, 95 or 99% sequence identity to SEQ ID No: 25 and/or

(c) the UTR sequence comprises or consists of SEQ ID NO: 3, 5, 7 or 39, or a sequence having at least 50, 60, 70, 75, 80, 85, 90, 95 or 99% sequence identity to SEQ ID NO: 3, 5, 7 or 39.

In another embodiment, the post-translational modification enzyme catalyzes the addition of oligosaccharide, galactose, fucose and/or sialic acid to a protein.

In another embodiment, the post-translational modification enzyme is GaIT, STT3D, FucT, a sialic acid synthesis enzyme or a transferase enzyme.

In another embodiment, the post-translational modification enzyme is GaIT, optionally human GaIT.

In another embodiment, the T-DNA region further comprises a second nucleic acid molecule encoding a recombinant protein.

In another embodiment, the recombinant protein is an antibody or fragment thereof. Optionally, the antibody or fragment thereof is trastuzumab or adalimumab.

In another embodiment, the recombinant protein is a therapeutic enzyme, optionally butyrylcholinesterase.

In another embodiment, the recombinant protein is a vaccine or a Virus Like Particle.

The disclosure also provides a kit comprising (a) a plant T-DNA vector as described herein and (b) a plant expression vector comprising a second nucleic acid molecule encoding a recombinant protein.

The disclosure also provides a genetically modified plant comprising a plant T-DNA vector as described herein.

In one embodiment, the plant or plant cell further comprises a nucleic acid sequence encoding a recombinant protein.

In another embodiment, the plant or plant cell is a tobacco plant or plant cell, optionally a Nicotiana plant or plant cell.

The disclosure also provides a method of obtaining a stable transgenic host plant comprising (a) introducing a plant T-DNA vector as described herein into a plant or plant cell and (b) selecting a transgenic plant with a stable expression of the first nucleic acid molecule. Also provided is a stable transgenic host plant obtained by the method. Optionally, the stable transgenic plant comprises a T-DNA insertion of the nucleic acid molecule at a single locus or at more than one locus. The transgenic plant may be heterozygous or homozygous for the T-DNA insertion.

The disclosure also provides a method of optimizing expression and/or glycosylation of a recombinant protein produced in a plant or plant cell, the method comprising:

(a) introducing into the plant or plant cell a plant T-DNA vector as described herein,

(b) introducing a second nucleic acid molecule encoding the recombinant protein into the plant or plant cell, and

(c) growing the plant or plant cell to obtain a plant that expresses the recombinant protein.

The disclosure also provides a method of increasing the amount of galactosylation on a recombinant protein produced in a plant or plant cell, the method comprising:

(a) introducing into the plant or plant cell a plant T-DNA vector as described herein,

(b) introducing a second nucleic acid molecule encoding the recombinant protein into the plant or plant cell, and

(c) growing the plant or plant cell to obtain a plant that expresses the recombinant protein, and wherein the post-translational modification enzyme is GaIT.

In one embodiment, the recombinant protein has a higher amount of galactosylation compared to the recombinant protein produced in a control plant or plant cell. Optionally, the control plant or plant cell is a plant or plant cell that expresses the post-translational modification enzyme behind a strong or intermediate strength promoter and/or is a wild-type plant or plant cell or a plant or plant cell genetically engineered for knock-out or knock-down of beta-1,2-xylosyltransferase and/or alpha-1,3-fucosyltransferase activities.

The disclosure also provides a method of increasing the amount of alpha-1,6-fucosylated glycans on a recombinant protein produced in a plant or plant cell, the method comprising:

(a) introducing into the plant or plant cell a plant T-DNA as described herein,

(b) introducing a second nucleic acid molecule encoding the recombinant protein into the plant or plant cell, and

(c) growing the plant or plant cell to obtain a plant that expresses the recombinant protein, and wherein the post-translational modification enzyme is an alpha-1,6-FucT.

In one embodiment, the recombinant protein has a higher amount of alpha-1,6-fucosylated glycans compared to the recombinant protein produced in a control plant or plant cell. Optionally, the control plant or plant cell is a plant or plant cell that expresses the post-translational modification enzyme behind a strong or intermediate strength promoter and/or is a wild-type plant or plant cell or a plant or plant cell genetically engineered for knock-out or knock-down of beta-1,2-xylosyltransferase and/or alpha-1,3-fucosyltransferase activities.

The disclosure also provides a method of decreasing the proportion of aglycosylation on recombinant protein produced in a plant or plant cell, the method comprising:

(a) introducing into the plant or plant cell a plant T-DNA vector as described herein,

(b) introducing a second nucleic acid molecule encoding the recombinant protein into the plant or plant cell, and

(c) growing the plant or plant cell to obtain a plant that expresses the recombinant protein,

and wherein the post-translational modification enzyme is STT3D.

In one embodiment, wherein the recombinant protein has a lower proportion of aglycosylated protein compared to the recombinant protein produced in a control plant or plant cell. Optionally, the control plant or plant cell is a plant or plant cell that expresses the post-translational modification enzyme behind a strong or intermediate strength promoter and/or is a wild-type plant or plant cell or a plant or plant cell genetically engineered for knock-out or knock-down of beta-1,2-xylosyltransferase and/or alpha-1,3-fucosyltransferase activities.

In another embodiment, introducing the plant T-DNA vector results in the stable integration of the nucleic acid molecule into the genome of the plant or plant cell. Optionally, the nucleic acid molecule is stably integrated at a single locus or at more than one locus in the genome of the plant or plant cell.

In another embodiment, the plant or plant cell is homozygous or heterozygous for the T-DNA insertion of the nucleic acid molecule.

In another embodiment, introducing the plant T-DNA vector results in the transient expression of the nucleic acid molecule in the plant or plant cell.

The disclosure also provides a recombinant protein produced by a plant or plant cell as described herein, or by a method as described herein.

Other features and advantages of the present disclosure will become apparent from the following detailed description. It should be understood, however, that the detailed description and the specific Examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The disclosure will now be described in relation to the drawings in which:

FIG. 1 shows schematic diagrams of plasmid pPFC0058 plus T-DNA regions of three other vivoXPRESS® expression vectors. (1A) Schematic of pPFC0058. LB, T-DNA left border sequence; term., transcriptional terminator; t′mab LC; trastuzumab light chain coding sequence; EE35S, double-enhancer Cauliflower Mosaic Virus (CaMV) 35S promoter; t′mab HC, trastuzumab heavy chain coding sequence; P19, tombusvirus P19 protein coding sequence; RB, T-DNA right border sequence; plasmid backbone. (1B) Schematic of T-DNA region of pPFC1433, including the double-enhancer version of the Cauliflower Mosaic Virus (CaMV) 35S promoter driving transcription of a chimeric human beta-1,4-galactosyltransferase (GaIT) coding sequence (SEQ ID Nos: 52 and 53; (STRASSER et al. 2009). This sequence includes 51 N-terminal amino acids from the cytoplasmic transmembrane stem region of a rat alpha-2,6-sialyltranferase (SEQ ID NO: 54 and 55). (1C) Schematic of T-DNA region of pPFC1434 including the same promoter driving transcription of a chimeric human alpha-1,6-fucosyltransferase (FucT) coding sequence (SEQ ID Nos: 21 and 22). This sequence includes a 39-aa putative signal peptide from a N. benthamiana FucT1 gene. (1D) Schematic of T-DNA region of pPFC1480 including the same promoter driving transcription of a Leishmania major oligosaccharyltransferase (STT3D).

FIG. 2 shows expression of trastuzumab antibody from vivoXPRESS® expression vector PFC0058 in transient co-expression treatments alone and in treatments involving PFC1506: double-enhancer 35S promoter (EE35S) driving transcription of a green fluorescent protein (GFP) coding sequence (CDS); PFC1433 (described in FIG. 1); PFC1458: a 4-nt frame-shift mutant of PFC1433 produced by Klenow fill-in of a unique Agel site at codons 64 and 65 of the hGaIT CDS; PFC1452: an expression vector involving the Arabidopsis ACT2 promoter (A_{N} et al. 1996) driving transcription of hGaIT (see schematic diagram, FIG. 4B); PFC1459, 4-nt Agel-mediated frame-shift mutant of PFC1452.

FIG. 3 shows expression of Ranibizumab antibody from vivoXPRESS® expression vector PFC2211 in transient co-expression treatments involving PFC1433; PFC1434, EE35S-FucT; PFC1480, EE35S-STT3D; and PFC1435, EE35S-P19.

FIG. 4 shows hGaIT expression vectors. T-DNA regions for vivoXPRESS vectors containing chimeric human galactosyltransferase under control of Cauliflower Mosaic Virus (CaMV) 35S promoter, or deletions thereof, or of Arabidopsis thaliana Act2 promoter. LB, functional 25-nt left border sequence; LB-rem., remnant Agrobacterium sequence associated with LB sequence; MCS, multi-cloning site; 35S_Enhancer, enhancer sequence of CaMV 35S promoter; 35S-basal P, basal promoter sequence of CaMV 35S promoter; 5′UTR, 5′ untranslated region; chimeric hGaIT CDS, coding sequence for chimeric human galactosyltransferase; rbcT, Rubisco terminator; RB, right border; ATG, methionine start-of-translation codon; E_rem., remnant enhancer sequence; P_rem., remnant basal promoter sequence. (A) pPFC1433, containing double-enhancer version of CaMV 35S promoter driving GaIT transcription; (B) pPFC1452, containing Act2 promoter driving GaIT; (C) pPFC1483, basal 35S promoter driving GaIT; (D) pPFC1484, 5′ UTR version 1 preceding GaIT; (E) pPFC1490, 5′ UTR version 2 preceding GaIT; (F) pPFC1492, 5′ UTR version 3 preceding GaIT; (G) pPFC1491, no-promoter/no-UTR preceding GaIT.

FIG. 5 shows expression of trastuzumab antibody in treatments involving hGaIT expression vectors described in FIGS. 4 and 5. This involved expression of trastuzumab from vivoXPRESS® vector pPFC0058 with simultaneous expression of hGaIT from one of seven vectors each having different promoters. Each treatment involved co-infiltration of N. benthamiana KDFX plants with two Agrobacterium strains: pPFC0058 and one hGaIT vector, each at an OD₆₀₀ of 0.2 according to published methods (GARABAGI et al. 2012a; GARABAGI et al. 2012b). Green leaf biomass was harvested (excluding leaf midribs) 7 days post infiltration (dpi). Trastuzumab amounts were measured using Pall::ForteBio BLItz instrumentation (https://www.fortebio.com/blitz.html), and expression is reported as mg trastuzumab/kg green biomass. Four biological replicates were performed for each treatment, and standard errors are presented on each histogram bar.

FIG. 6 shows galactosylation of trastuzumab for the experimental treatments of FIG. 5. This involved SDS-PAGE (reduced) and Western blot analysis of trastuzumab samples purified using antibody spintrap columns from GE Healthcare (catalog number 28-4083-47). Samples were applied to 10% SDS-PAGE gels, electrophoresed and stained or transferred to blotting membrane according to the method of Grohs et al. (GRos et al. 2010). The left side of the figure shows a western immunoblot and the right side shows equal loading of antibody samples on SDS-PAGE gel. The western immunoblot was probed using biotinylated Ricinus communis Agglutinin I (RCA; Vector Labs, catalog number B-1085), followed by horseradish peroxidase conjugated streptavidin (HRP; BioLegend, catalog number 405210); chemiluminescent signal development used the SuperSignal West Pico Chemiluminescent Substrate (ThermoFisher, catalog number 34080) and standard procedures recommended by commercial vendors. Vector treatments are given above gel and immunoblot images. MW given on left in kilo Daltons (kD). Left, immunoblot probed with RCA lectin; right, Coomassie blue stained SDS-PAGE gel.

FIG. 7 shows schematic diagrams for T-DNA regions of four alpha-1,6-fucosyltransferase expression vectors. The amino acid sequence of a putative signal peptide (SP) from the Nicotiana benthamiana fucosyltransferase-1 (GenBank: ABU48860.1) was added to the 547 C-terminal amino acids of human alpha-1,6-fucosyltransferase (hFucT; NCBI Reference Sequence: NP_835368.1) and codon-optimization for expression in Nicotiana benthamiana was determined (undisclosed PlantForm procedures). This sequence was synthesized and assembled into expression vectors downstream of promoters or without a promoter using standard procedures. T-DNA regions for vivoXPRESS vectors containing chimeric human alpha-1,6-fucosyltransferase under control of Cauliflower Mosaic Virus (CaMV) 35S promoter, or deletions thereof, or of Arabidopsis thaliana Act2 promoter are provided. LB, functional 25-nt left border sequence; LB-rem., remnant Agrobacterium sequence associated with LB sequence; MCS, multi-cloning site; 35S_Enhancer, enhancer sequence of CaMV 35S promoter; 35S-basal P, basal promoter sequence of CaMV 35S promoter; 5′UTR, 5′ untranslated region; FT-FUT8, chimeric hFucT, coding sequence; rbcT, Rubisco terminator; RB, right border; ATG, methionine start-of-translation codon; E_rem., remnant enhancer sequence; P_rem., remnant basal promoter sequence. (A) pPFC1434, containing double-enhancer version of CaMV 35S promoter driving hFucT transcription; (B) pPFC1455, containing Act2 promoter driving hFucT; (C) pPFC1485, basal 35S promoter driving hFucT; (D) pPFC1486, 5′ UTR preceding hFucT. See also Table 4 which details the sequence differences in the LB to ATG start of translation codon regions between the four FucT plasmids of FIG. 7 and the four related hGaIT plasmids of FIG. 4.

FIG. 8 shows expression of trastuzumab antibody in treatments involving hFucT expression vectors described in FIG. 7. As with FIG. 5, this involved expression of trastuzumab from pPFC0058 with simultaneous expression of hFucT from one of four vectors each having different promoters. Each treatment involved co-infiltration of N. benthamiana KDFX plants with two Agrobacterium strains: pPFC0058 and one hFucT vector, each at an OD₆₀₀ of 0.2 according to published methods (GARABAGI et al. 2012a; GARABAGI et al. 2012b). Green leaf biomass was harvested (excluding leaf midribs) 7 days post infiltration (dpi). Trastuzumab amounts were measured using Pall:ForteBio BLItz instrumentation (https://www.fortebio.com/blitz.html), and expression is reported as mg trastuzumab/kg green biomass. Four biological replicates were performed for each treatment, and standard errors are presented on each histogram bar.

FIG. 9 shows alpha-1,6-fucosylation of trastuzumab for the experimental treatments of FIG. 9. As with FIG. 6, this involved SDS-PAGE (reduced) and Western blot analysis of trastuzumab samples purified using antibody spintrap columns from GE Healthcare (catalog number 28-4083-47). Samples were applied to 10% SDS-PAGE gels, electrophoresed and stained or transferred to blotting membrane according to the method of Grohs et al. (GRoHs et al. 2010). The right side of the figure shows a western immunoblot and the left side shows equal loading of antibody samples on SDS-PAGE gel. The western immunoblot was probed using biotinylated Aleuria aurantia Lectin (AAL; Vector Labs, catalog number B-1395), followed by horseradish peroxidase conjugated streptavidin (HRP; BioLegend, catalog number 405210); chemiluminescent signal development used the SuperSignal West Pico Chemiluminescent Substrate (ThermoFisher, catalog number 34080) and standard procedures recommended by commercial vendors. Vector treatments are given above gel and immunoblot images. MW given on left in kilo Daltons (kD). Left, immunoblot probed with RCA lectin; right, Coomassie blue stained SDS-PAGE gel.

FIG. 10 shows STT3D expression vectors. T-DNA regions for vivoXPRESS vectors containing coding sequence for Leishmania major STT3D oligosaccharyltransferase under control of Cauliflower Mosaic Virus (CaMV) 35S basal promoter, or deletions thereof. LB, functional 25-nt left border sequence; LB-rem., remnant Agrobacterium sequence associated with LB sequence; MCS, multi-cloning site; E-rem., enhancer sequence remnant of CaMV 35S promoter; 35S-basal P, basal promoter sequence of CaMV 35S promoter; 5′UTR, 5′ untranslated region; STT3D CDS, STT3D coding sequence; nosT, nopaline synthase terminator; RB, right border; ATG, methionine start-of-translation codon; P_rem., remnant basal promoter sequence. (A) pPFC1487, containing basal 35S promoter driving STT3D transcription; (B) pPFC1488, 5′ UTR preceding STT3D; (C) pPFC1494, no-promoter/no-UTR preceding STT3D. See also Table 6 which details the sequence differences in the LB to ATG start of translation codon regions between the three STT3D plasmids of FIG. 10 and the three related hGaIT plasmids of FIG. 4.

FIG. 11 shows expression of trastuzumab antibody in treatments involving STT3D expression vectors described in FIG. 10. As with FIG. 5, this involved expression of trastuzumab from pPFC0058 with simultaneous expression of STT3D from one of three vectors each having different promoters or entirely lacking a promoter and 5′UTR. Each treatment involved co-infiltration of N. benthamiana KDFX plants with two Agrobacterium strains: pPFC0058 and one STT3D vector, each at an OD₆₀₀ of 0.2 according to published methods (GARABAGI et al. 2012a; GARABAGI et al. 2012b). Green leaf biomass was harvested (excluding leaf midribs) 7 days post infiltration (dpi). Trastuzumab amounts were measured using Pall::ForteBio BLItz instrumentation (https://www.fortebio.com/blitz.html), and expression is reported as mg trastuzumab/kg green biomass. Three biological replicates were performed for each treatment, and standard errors are presented on each histogram bar. n=3.

FIG. 12 shows the proportion of aglycosylated trastuzumab heavy chains (HC) as determined for the experiment of FIG. 11, for which weak cation exchange high performance liquid chromatography (WCX-HPLC) was used to determine the proportion of glycosylated, hemi-glycosylated, and aglycosylated monoclonal antibody (mAb). 10 μL of sample at −1.8 mg/mL was injected at a flow rate of 1 mL/min into an Agilent Bio Mab, NPS, SS column (4.6×250 mm, 5 μm, P/N 5190-2405; Agilent). Agilent ChemStation software was used to calculate the peak areas of these peaks, the percent aglycosylated HC was then summarized as shown in the figure.

FIG. 13 shows schematic diagrams of three vivoXPRESS® expression vectors designed for development of stable transgenic plant lines expressing (A) hGaIT from a promoter and 5′UTR-lacking gene (PFC1403); (B) STT3D from a basal-35S promoter (PFC1404); and (C) hGaIT from a promoter and 5′UTR-lacking gene along with STT3D from a basal-35S promoter (PFC1405). LB, T-DNA left border region; nosT, nopaline synthase gene terminator sequence; PFC synthetic sequence: PAT, synthetic DNA sequence for phosphinothricin acetyl transferase; nosP, nopaline synthase gene promoter sequence; “no promoter, no UTR” hGaIT chimeric gene, gene sequence for hGaIT lacking promoter and UTR sequences; RB, T-DNA right border sequence; rbcT, ribulose-1,5-bisphosphate carboxylase-oxidase gene terminator sequence; PFC synthetic cds (coding sequence): hGaIT (SEQ ID No: 17); CTS, cytoplasmic transmembrane stem region sequence; PFC synthetic cds: LmSTT3D (SEQ ID No: 21); CaMV basal 35S P, basal sequence of cauliflower mosaic virus 35S promoter; N. benth. rep., repetitive DNA sequence taken from genome of N. benthamiana.

FIG. 14 shows an RCA lectin-based screen for transgenic plant line(s) having GaIT activity. Primary transgenic plants produced with vivoXPRESS® T-DNA vector PFC1403 were self-pollinated and T₁ seed sets were collected. Two to six T₁ plants from 20 such seed sets were grown to 5-6 weeks of age and infiltrated with trastuzumab vector PFC0058. Antibody was purified 7 days post-infiltration by Protein A (SpinTrap) and 3 μg samples were electrophoresed under denaturing conditions through SDS-PAGE gels, which were either stained with Coomassie blue (to confirm equivalent loading; left panel) or blotted to PVDF membrane and probed with RCA lectin for presence galactose due to post-translational modification (right panel), as described in Methods. To each gel and blot, antibody produced in KDFX plants was applied as a negative control; antibody produced in KDFX plants treated with vector PFC1403 for transient co-expression of GaIT was applied as a positive control. T₁ sibling plants from primary transgenic plant number 1403-25 produced antibody that was galactosylated, as seen in the right panel above. Two more such sets of stained-gels and probed-blots were produced; however, these are not presented as no other T₁ sibling plant families produced antibody that was galactosylated.

FIGS. 15A and 15B show Coomassie blue-stained SDS-PAGE gels (left) and RCA lectin-probed western blots (right) of trastuzumab antibody purified from T2 sibling plants of self pollinated T1 transgenic plants 1403-25-xx (where xx=01, 07, 11, 16, 19, 21, 24, 25, 54, 55). KDFX plant sample (negative control) and positive control sample from T1 sibling plants from T0 plant 1403-25 (from experiment shown in FIG. 14) were applied to each gel and on each western blot; also, a molecular weight size standard is present in the left-most lane of each Coomassie blue-stained gel.

### DETAILED DESCRIPTION

Better control for addition of sugars to valuable therapeutic proteins can be achieved by varying the expression strengths of genes that encode enzymes responsible for key glycosylation activities in plants genetically engineered for this purpose. The present disclosure describes T-DNA vectors with engineered 5′ sequences upstream of a post-translational modification enzyme coding sequence. These vectors allow control of the transcriptional activity of the post-translational modification enzyme.

The vectors described herein can be used for transient expression of the encoded post-translational modification enzyme in plants which are further engineered to produce recombinant proteins. These vectors can also be used for the generation of stable transgenic host plants that express transgene-encoded post-translational modification enzymes with reduced activities. In both cases, the goal is to produce recombinant proteins in plants with defined glycosylation.

### Compositions of Matter

### Vectors

Accordingly, the present disclosure provides plant expression vectors comprising a nucleic acid molecule encoding a post-translational modification enzyme, wherein the vector lacks a traditional promoter sequence for the nucleic acid molecule.

The present disclosure also provides plant expression vectors comprising a nucleic acid molecule encoding a post-translational modification enzyme, wherein the vector lacks both a traditional promoter sequence and a 5′ untranslated region (5′UTR) sequence for the nucleic acid molecule.

As used herein, the term “vector” or “expression vector” means a nucleic acid molecule, such as a plasmid, comprising regulatory elements and a site for introducing transgenic DNA, which is used to introduce the transgenic DNA into a plant or plant cell. Regulatory elements include promoters, 5′ and 3′ untranslated regions (UTRs) and terminator sequences or truncations thereof.

Various vectors useful for expression in plants are well known in the art. Examples of plant expression vectors contemplated by the present disclosure include, but are not limited to, T-DNA expression vectors. T-DNA expression vectors are based on the Ti plasmid of Agrobacterium tumefaciens. A T-DNA expression vector includes both a T-DNA region and a “maintenance” region required for maintaining the plasmid in the Agrobacterium cell line. The maintenance region consists of one or more selectable marker genes (beta lactamase, neomycin phosphotransferase, others); one or more origins of replication (on). The T-DNA region is a stretch of DNA flanked by Left Border and Right Border sequences at either end, and which can integrate, in full or in part, into the plant genome.

Specific examples of vector systems useful in the methods of the present disclosure include, but are not limited to, the Magnifection (Icon Genetics), pEAQ (Lomonosoff), Geminivirus (Arizona State U.), vivoXPRESS® vector systems, and vector systems based on pBIN19 (BEVAN 1984).

In one embodiment, the T-DNA region comprises a nucleic acid molecule encoding a protein of interest.

In one embodiment, the protein of interest is a post-translational modification enzyme.

As used herein, the term “nucleic acid molecule” means a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present disclosure may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

As used herein, the term “post-translational modification enzyme” refers to an enzyme which has post-translational modification activity. Post-translational modification of proteins refers to the chemical changes proteins may undergo after translation. Post-translational modification enzymes can catalyze these changes by recognizing specific target sequences in specific proteins. Examples of post-translational modifications include, but are not limited to, the addition of oligosaccharides, galactose, fucose and/or sialic acid to the translated protein.

In one embodiment of the disclosure, the post-translational modification enzyme is beta-1,4-galactosyltransferase (GaIT), a single subunit protist oligosaccharyltransferase (OST), STT3D, alpha-1,6-fucosyltransferase (FucT), mannosidase I (MI), mannosidase II (MII), β-1,2-GlcNAc transferase I (GnTI), 8-1,2-GlcNAc transferase II (GnTII), UDP-Galactose transporter (HuGT1), a sialic acid synthesis enzyme or a transferase enzyme. The post-translational modification enzyme may be obtained from any species or source.

The term “GaIT” as used herein refers to a galactosyltransferase protein which is encoded by a GaIT gene. The term “GaIT” includes GaIT from any species or source. The term also includes sequences that have been modified from any of the known published sequences of GaIT genes or proteins. The GaIT gene or protein may have any of the known published sequences for GaIT which can be obtained from public sources such as GenBank. The human genome includes a number of GaIT genes including human beta-1,4-galactosyltransferase. An example of the human sequence for the functional domain (enzymatic domain) of beta-1,4-galactosyltransferase include the amino acid sequence set out in SEQ ID NO: 16. “GaIT” also refers to a protein comprising, consisting of, or consisting essentially of, an amino acid sequence having at least 50, 60, 70, 75, 80, 85, 90, 95 or 99% sequence identity to SEQ ID NO: 16, while retaining GaIT function.

As used herein, the term “GaIT” includes a chimeric protein comprising GaIT, or a functional domain thereof. An example of a chimeric protein comprising GaIT is set out in SEQ ID NO: 17.

SEQ ID NO: 17 contains a 332 amino acid sequence from the C-terminus of the Homo sapiens beta-1,4-galactosyltransferase 1 (NCBI Reference Sequence: NP_001488.2). This 332 amino acid sequence is the functional (i.e., enzymatic) domain of this protein. The coding sequence for the first 66 amino acids of the human protein is not incorporated into the chimeric hGaIT coding sequence; instead, the coding sequence for the rat alpha 2,6-sialyltransferase 1 CTS (cytoplasmic transmembrane stem) region (NCBI Reference Sequence: NP_001106815.1) has been incorporated to encode the N-terminal 51 amino acids of the chimeric protein. Accordingly, in another embodiment, the post-translational modification enzyme is a protein comprising, consisting of, or consisting essentially of, an amino acid sequence having at least 50, 60, 70, 75, 80, 85, 90, 95 or 99% sequence identity to SEQ ID NO: 17, while retaining GaIT function.

The term “OST” as used herein refers to an oligosaccharyltransferase which is encoded by an OST gene. In one embodiment, the term “OST” includes OST from any species or source. The term also includes sequences that have been modified from any of the known published sequences of OST genes or proteins. The OST gene or protein may have any of the known published sequences for DST's which can be obtained from public sources such as GenBank. In one embodiment, the OST protein is STT3D from Leishmania major (LmSTT3D; GenBank XP_003722509). See also Nasab et al., 2008. An example of the Leishmania sequence for STT3D includes the amino acid sequence set out in SEQ ID NO: 18 and the nucleic acid sequence set out in SEQ ID: 19. “STT3D” also refers to a protein having at least 50, 60, 70, 75, 80, 85, 90, 95 or 99% sequence identity to SEQ ID NO: 18, while retaining STT3D function. The STT3D gene includes sequences having at least 50, 60, 70, 75, 80, 85, 90, 95 or 99% sequence identity to SEQ ID NO: 19, where the sequence encodes for a protein having STT3D function. As used herein, the term “STT3D” includes a chimeric protein comprising STT3D, or a functional domain thereof.

The term “FucT” as used herein refers to a fucosyltransferase protein which is encoded by a FucT gene. The term “FucT” includes FucT from any species or source and includes alpha-1,2-fucosyltransferases, alpha-1,3-fucosyltransferases, alpha-1,4-fucosyltransferases and alpha-1,6-fucosyltransferases. The term also includes sequences that have been modified from any of the known published sequences of FucT genes or proteins. The FucT gene or protein may have any of the known published sequences for FucT which can be obtained from public sources such as GenBank. The human genome includes a number of FucT genes including human fucosyltransferase. An example of a human fucosyltransferase is Homo sapiens alpha-1,6-fucosyltransferase isoform a (NCBI: NP_835368.1). “FucT” also refers to a protein having at least 50, 60, 70, 75, 80, 85, 90, 95 or 99% sequence identity to Homo sapiens alpha-1,6-fucosyltransferase isoform a (NCBI: NP_835368.1), while retaining FucT function.

As used herein, the term “FucT” includes a chimeric protein comprising FucT, or a functional domain thereof. An example of a chimeric protein comprising FucT is set out in SEQ ID NO: 20.

SEQ ID NO: 20 contains a 547 amino acid sequence from the C-terminus of the Homo sapiens alpha-1,6-fucosyltransferase isoform a (NCBI: NP_835368.1). This 547 amino acid sequence is the functional (i.e., enzymatic) domain of this protein. The coding sequence for the first 29 amino acids of the human protein is not incorporated into the chimeric FucT coding sequence; instead, the coding sequence for the signal peptide of the N. benthamiana fucosyltransferase 1 (NCBI: ABU48860.1) has been incorporated to encode the N-terminal 39 amino acids of the chimeric protein.

In one embodiment, the protein of interest is a protein that has a deleterious effect on plant growth and/or metabolism (i.e., a protein toxic to plants). In another embodiment, the protein of interest is a protease enzyme. In another embodiment, the protein of interest is a protein with regulatory function (for example, a transcriptional activator), a substrate transporter, a component of a plant stress response system (for example a heat shock chaperone), or an epigenetic regulator (for example, a histone methyl transferase/demethylase or a DNA methyl transferase/demethylase). In another embodiment, the protein of interest is a transgene encoded protein involved in genome editing, an RNA-guided DNA endonuclease associated with the CRISPR adaptive immunity system (for example, Cas9), a meganuclease, a zinc finger nuclease, or a transcription activator-like effector based nuclease (TALEN).

As described herein, the inventors have shown that engineering the 5′ sequences upstream of a post-translational modification enzyme can result in reduced expression strength and therefore resulting in reduced activities of these enzymes. In particular, the inventors have shown that a T-DNA vector where the vector lacks, or has an absence of, a traditional promoter sequence that would normally direct transcription of the post-translational modification enzyme coding sequence leads to reduced, but not absent, expression of the enzyme. The inventors have shown that a T-DNA vector where the vector has only a small fragment (for example, 3, 4, 5, 6, 7, 8, 9, 10, 15 or 20 contiguous base pairs) of a promoter sequence encoding the post-translational modification enzyme leads to reduced expression of the enzyme. Reduced activity of post-translational modification enzymes can help to optimize glycosylation of recombinant protein produced in plants.

Some post-translational modification enzymes, when expressed without traditional promoters, may still require further weakening of expression. In such cases, it is possible to remove the untranslated region (UTR; i.e., the DNA sequence 5′ of the ATG start of translation codon to the start of transcription). In these cases, the ATG start of translation codon is positioned immediately adjacent to either the left border (LB) or the right border (RB) regions of the T-DNA vector.

In one embodiment of the present disclosure, a T-DNA vector is provided having a T-DNA region. As used herein, the term “T-DNA region” refers to a stretch of DNA flanked by “Left border (LB)” and “Right border (RB)” sequences at either end and which can integrate into the plant genome.

As used herein, the terms “left border sequence” or “LB sequence” (also referred to herein as a “functional LB sequence”) and “right border sequence” or “RB sequence” (also referred to herein as a “functional RB sequence”) refers to short sequences, for example 20-30, optionally 23-26 or 25 bp sequences, that flank the T-DNA region. The LB and RB sequences are the cis elements required to direct T-DNA processing; any DNA between the LB and RB sequences may be transferred to the plant cell. The LB and RB sequences can comprise similar, although not necessarily identical, sequences. LB and RB sequences are well-known in the art (see for example, Yadav, N S et al., 1982 and Zupan and Zampbryski, 1995). In one embodiment, the LB sequence comprises or consists of a sequence as set out in SEQ ID No: 1 or a sequence having at least 50, 60, 70, 75, 80, 85, 90, 95 or 99% sequence identity to SEQ ID No: 1. In another embodiment, the RB sequence comprises or consists of a sequence as set out in SEQ ID No: 25 or a sequence having at least 50, 60, 70, 75, 80, 85, 90, 95 or 99% sequence identity to SEQ ID Nos: 25. In another embodiment, the LB or RB sequence is a border sequence provided in Slightom et al (1986, The Journal of Biological Chemistry 261, 108-121), the contents of which is incorporated herein in its entirety.

The term “left border region” and “right border region” as used herein refers to a sequence that includes the LB or RB sequence, respectively, and optionally also includes left border or right border associated sequences and/or at least one multiple cloning site. For example, with respect to vector PFC1450, the left border sequence is SEQ ID NO: 14/SEQ ID NO: 23 and the left border region includes the LB sequence as well as 73 nucleotides of LB associated sequence and a multiple cloning site (SEQ ID NO: 56). With respect to vectors PFC1491 and PFC1494, the left border region consists of only the LB sequence (SEQ ID NO: 14/SEQ ID NO: 23). In the vectors described herein, the T-DNA region comprises a nucleic acid sequence encoding a post-translational modification enzyme. The post-translational modification enzyme is optionally downstream of the LB or the RB sequence.

The vectors described herein do not contain a traditional promoter sequence driving the expression of the post-translational modification enzyme. As is well known in the art, a “promoter” is a promoter is a region of DNA that initiates transcription of a particular gene. As used herein, the expression “traditional promoter” refers to a known promoter sequence. Rather, in one embodiment, in the vectors described herein, the vector has an absence of any promoter sequence driving the expression of the post-translational modification enzyme. In another embodiment, the vector comprises a fragment of a promoter sequence. Further, some of the vectors described herein also do not contain an untranslated region (UTR) on the 5′ side of the nucleic acid sequence encoding a post-translational modification enzyme.

Thus, in one embodiment, the T-DNA region comprises a nucleic acid sequence encoding a post-translational modification enzyme that is directly adjacent to the “left border (LB)” or “right border (RB)” sequence. As used herein, the term “directly adjacent” means that there are no intervening nucleic acids between the two sequences. In these embodiments, the ATG start of translation codon of the nucleic acid sequence encoding a post-translational modification enzyme is positioned immediately adjacent to either the left border (LB) or the right border (RB) sequence. Examples of vectors where the nucleic acid sequence encoding a post-translational modification enzyme is directly adjacent to the border sequence include PFC1491 and PFC1494. In another embodiment, the T-DNA region comprises a nucleic acid sequence encoding a post-translational modification enzyme that is separated from the left border (LB) or right border (RB) sequence by 10 or less, 9 or less, 8 or less, 7 or less, 6 or less or 5 or less nucleotides. In a further embodiment, the T-DNA region comprises a nucleic acid sequence encoding a post-translational modification enzyme that is separated from the left border (LB) or right border (RB) sequence by one or more restriction sites. For example, vectors PFC1405 and PFC1403 have a 6-nt HindIII site between the RB sequence and the ATG start site.

In another embodiment, the T-DNA region comprises an untranslated region (UTR) on the 5′ side of the nucleic acid sequence encoding a post-translational modification enzyme. This untranslated region is also referred to as a 5′UTR sequence or a leader sequence. In some embodiments, the UTR is directly adjacent to, and upstream of the post-translational modification enzyme. Examples of vectors where the UTR is directly adjacent to, and upstream of, the post-translational modification enzyme include PFC1484, PFC1486, PFC1488, PFC1490 and PFC1492.

Examples of 5′ UTR sequences include the CaMV 35S UTR (GenBank Sequence ID: gi|58815|V00140.1; SEQ ID NO: 59), the Arabidopsis Act2 UTR (GenBank Sequence ID: U41998.1; SEQ ID NOs: 60 and 61) and the Arabidopsis Act8 UTR (GenBank Sequence ID: ATU42007; SEQ ID NOs: 62 and 63). In one embodiment, the UTR sequence comprises or consists of the sequence set out as SEQ ID NO: 3, 5, 7 or 39, or a sequence having at least 50, 60, 70, 75, 80, 85, 90, 95 or 99% sequence identity to SEQ ID NO: 3, 5, 7 or 39.

In other embodiments, the nucleic acid encoding the post-translational modification enzyme or the 5′UTR sequence is separated from the left or right border sequence by an upstream sequence of 100 base pairs or less. In one embodiment, the nucleic acid encoding post-translational modification enzyme or the 5′UTR sequence is separated from the left or right border sequence by an upstream sequence of 90, 80, 70, 60, 50, 40, 30, 20, 15, 10, 6 or 5 base pairs or less. This, in one embodiment, the T-DNA region comprises an upstream sequence.

In one embodiment, the upstream sequence comprises or consists of at least one fragment of a promoter. As used herein, the term “fragment of a promoter” refers to no more than 3, 4, 5, 6, 7, 8, 9, 10, 15 or 20 contiguous nucleic acid residues of a promoter sequence. The fragment is optionally from the 5′ end or 3′ end of the promoter sequence, or from any intervening sequence. The promoter is optionally the 35S promoter or the ACT2 promoter. On some embodiments, the upstream sequence comprises or consists of at least one, at least two or at least three fragments of a promoter. The fragments may be of identical or differing sequences.

In one embodiment, the upstream sequence comprises or consists of a fragment of the 35S basal promoter as set out in SEQ ID No: 2 or 10, or a sequence having at least 50, 60, 70, 75, 80, 85, 90, 95 or 99% sequence identity to SEQ ID NO: 2 or 10. In another embodiment, the upstream sequence comprises or consists of a fragment of the 35S basal promoter as set out in SEQ ID NO: 37, or a sequence having at least 50, 60, 70, 75, 80, 85, 90, 95 or 99% sequence identity to SEQ ID NO: 37.

In another embodiment, the upstream sequence comprises or consists of SEQ ID NO: 2 or SEQ ID NO: 10 or a sequence having at least 50, 60, 70, 75, 80, 85, 90, 95 or 99% to SEQ ID NO: 2 or 10.

Examples of vectors where the nucleic acid encoding post-translational modification enzyme or the 5′UTR sequence is separated from the border region by an upstream sequence comprising a fragment of a promoter include PFC1484, PFC1486, PFC1488, PFC1490 and PFC1492.

In one embodiment, a T-DNA vector is provided comprising a sequence comprising, consisting of, or consisting essentially of, from 5′ to 3′ (i) SEQ ID NO: 1, or a sequence having at least 50, 60, 70, 75, 80, 85, 90, 95 or 99% to SEQ ID NO:1, (ii) SEQ ID NO: 2, or a sequence having at least 50, 60, 70, 75, 80, 85, 90, 95 or 99% to SEQ ID NO: 2, (iii) SEQ ID NO: 3 or a sequence having at least 50, 60, 70, 75, 80, 85, 90, 95 or 99% to SEQ ID NO: 3, and (iv) a sequence encoding a post-translational modification enzyme, optionally GaIT. In one embodiment, the sequence encoding GaIT is SEQ ID NO: 17, or a sequence having at least 50, 60, 70, 75, 80, 85, 90, 95 or 99% to SEQ ID NO: 17. An example of such a T-DNA vector is PFC1484.

In another embodiment, a T-DNA vector is provided comprising a sequence comprising, consisting of, or consisting essentially of, from 5′ to 3′ (i) SEQ ID NO: 1, or a sequence having at least 50, 60, 70, 75, 80, 85, 90, 95 or 99% to SEQ ID NO:1 (ii) SEQ ID NO: 2, or a sequence having at least 50, 60, 70, 75, 80, 85, 90, 95 or 99% to SEQ ID NO: 2, (iii) SEQ ID NO: 5 or a sequence having at least 50, 60, 70, 75, 80, 85, 90, 95 or 99% to SEQ ID NO: 5, and (iv) a sequence encoding a post-translational modification enzyme, optionally FucT. In one embodiment, the sequence encoding FucT is SEQ ID No: 21, or a sequence having at least 50, 60, 70, 75, 80, 85, 90, 95 or 99% to SEQ ID NO: 21. An example of such a T-DNA vector is PFC1486.

In another embodiment, a T-DNA vector is provided comprising a sequence comprising, consisting of, or consisting essentially of, from 5′ to 3′ (i) SEQ ID NO: 57, or a sequence having at least 50, 60, 70, 75, 80, 85, 90, 95 or 99% to SEQ ID NO:57, (ii) SEQ ID NO: 7 or a sequence having at least 50, 60, 70, 75, 80, 85, 90, 95 or 99% to SEQ ID NO: 7, and (iii) a sequence encoding a post-translational modification enzyme, optionally STT3D. In one embodiment, the sequence encoding STT3D is SEQ ID NO: 19, or a sequence having at least 50, 60, 70, 75, 80, 85, 90, 95 or 99% to SEQ ID NO: 19. An example of such a T-DNA vector is PFC1488.

In another embodiment, a T-DNA vector is provided comprising a sequence comprising, consisting of, or consisting essentially of, from 5′ to 3′ (i) SEQ ID NO: 9, or a sequence having at least 50, 60, 70, 75, 80, 85, 90, 95 or 99% to SEQ ID NO:9, and (ii) SEQ ID NO: 10, or a sequence having at least 50, 60, 70, 75, 80, 85, 90, 95 or 99% to SEQ ID NO: 10, (iii) SEQ ID NO: 3 or a sequence having at least 50, 60, 70, 75, 80, 85, 90, 95 or 99% to SEQ ID NO: 3, and (iv) a sequence encoding a post-translational modification enzyme, optionally GaIT. In one embodiment, the sequence encoding GaIT is SEQ ID NO: 17, or a sequence having at least 50, 60, 70, 75, 80, 85, 90, 95 or 99% to SEQ ID NO: 17. An example of such a T-DNA vector is PFC1490.

In another embodiment, a T-DNA vector is provided comprising a sequence comprising, consisting of, or consisting essentially of, from 5′ to 3′ (i) SEQ ID NO: 12, or a sequence having at least 50, 60, 70, 75, 80, 85, 90, 95 or 99% to SEQ ID NO:12, (ii) SEQ ID NO: 10, or a sequence having at least 50, 60, 70, 75, 80, 85, 90, 95 or 99% to SEQ ID NO: 10, (iii) SEQ ID NO: 3 or a sequence having at least 50, 60, 70, 75, 80, 85, 90, 95 or 99% to SEQ ID NO: 3, and (iv) a sequence encoding a post-translational modification enzyme, optionally GaIT. In one embodiment, the sequence encoding GaIT is SEQ ID NO: 17, or a sequence having at least 50, 60, 70, 75, 80, 85, 90, 95 or 99% to SEQ ID NO: 17. An example of such a T-DNA vector is PFC1492.

In another embodiment, a T-DNA vector is provided comprising a sequence comprising, consisting of, or consisting essentially of, from 5′ to 3′ (i) SEQ ID NO: 14, or a sequence having at least 50, 60, 70, 75, 80, 85, 90, 95 or 99% to SEQ ID NO:14 and (ii) a sequence encoding GaIT. In one embodiment, the sequence encoding GaIT is SEQ ID NO: 17, or a sequence having at least 50, 60, 70, 75, 80, 85, 90, 95 or 99% to SEQ ID NO: 17. An example of such a T-DNA vector is PFC1491.

In another embodiment, a T-DNA vector is provided comprising a sequence comprising, consisting of, or consisting essentially of, from 5′ to 3′ (i) SEQ ID NO: 14, or a sequence having at least 50, 60, 70, 75, 80, 85, 90, 95 or 99% to SEQ ID NO:14, and (ii) a sequence encoding a post-translational modification enzyme, optionally STT3D. In one embodiment, the sequence encoding STT3D is SEQ ID NO: 19, or a sequence having at least 50, 60, 70, 75, 80, 85, 90, 95 or 99% to SEQ ID NO: 19. An example of such a T-DNA vector is PFC1494.

In one embodiment, the T-DNA region is oriented from the LB sequence to the RB sequence, where the LB sequence is upstream of the RB sequence. In another embodiment, the T-DNA region is oriented from the RB sequence to the LB sequence, where the RB sequence is upstream of the LB sequence. Examples of T-DNA vectors oriented with the RB sequence upstream of the LB region sequence P1403 and P1405. This approach (RB sequence upstream of the LB sequence) can be particularly useful when using the vectors to generate stable plant lines. T-DNAs are directionally inserted into the genome, such that the RB sequence is inserted first and the remainder follows. Published data show that there can be truncations towards the LB sequence end. Thus without being bound by theory, having the RB sequence adjacent to, or close to, the ATG start codon, may help to promote the integrity of the integration.

In another embodiment, a T-DNA vector is provided comprising a sequence comprising, consisting of, or consisting essentially of, from 5′ to 3′ (i) SEQ ID NO: 91, or a sequence having at least 50, 60, 70, 75, 80, 85, 90, 95 or 99% to SEQ ID NO: 91, (ii) SEQ ID No: 89 or a sequence having at least 50, 60, 70, 75, 80, 85, 90, 95 or 99% to SEQ ID NO: 89, and (iii) a sequence encoding a post-translational modification enzyme, optionally GaIT. In such an embodiment, the sequence encoding GaIT comprises SEQ ID NO: 88 plus SEQ ID No: 87 or a sequence having at least 50, 60, 70, 75, 80, 85, 90, 95 or 99% to SEQ ID NO: 88 plus a sequence having at least 50, 60, 70, 75, 80, 85, 90, 95 or 99% to SEQ ID NO: 87. Examples of such T-DNA vectors include PFC1403 and PFC1405.

The T-DNA region optionally includes other regulatory elements, including but not limited to, a terminator sequence for the nucleic acid sequence encoding a post-translational modification enzyme, a 5′ untranslated region (5′UTR), a Kozak box, a TATA box, a CAAT box and one or more enhancers and/or a 3′ UTR. In some embodiments, the T-DNA region comprises a selectable marker useful for making stable transgenic plants (for example, a marker conferring phosphinothricin acetyl transferase (PAT) resistance, also known as Basta® resistance).

In another embodiment, the T-DNA region contains a nucleic acid sequence comprising coding sequences for more than one post-translational modification enzyme between the LB and RB sequences, optionally two or three nucleic acid molecule encoding post-translational modification enzymes. In such an embodiment, the post-translational modification enzymes may be the same or a different enzyme. In such an embodiment, the expression of at least one nucleic acid molecule is not driven by a traditional promoter sequence, but instead has an upstream sequence as described herein.

In one embodiment, in addition to the post-translational modification enzyme, the T-DNA region further comprises a sequence that encodes another recombinant protein, which can be expressed in and isolated from a plant or plant cell. In other embodiments, a second nucleic acid molecule that encodes a recombinant protein is expressed from a separate vector.

As used herein, the term “recombinant protein” means any polypeptide that can be expressed in a plant cell, wherein said polypeptide is encoded by DNA introduced into the plant cell via use of an expression vector.

In one embodiment, the recombinant protein is an antibody or antibody fragment. In a specific embodiment, the antibody is trastuzumab or a modified form thereof, consisting of 2 heavy chains (HC) and 2 light chains (LC). Trastuzumab (Herceptin® Genentech Inc., San Francisco, Calif.) is a humanized murine immunoglobulin G 1 K antibody that is used in the treatment of metastatic breast cancer.

In another embodiment, the antibody is adalimumab (trade name Humira®).

Where the recombinant protein is an antibody or antibody fragment, a nucleic acid encoding the heavy chain and a nucleic acid encoding the light chain may be present in the same vector or on different vectors. As used herein, the term “antibody fragment” includes, without limitation, Fab, Fab′, F(ab′)₂, scFv, dsFv, ds-scFv, dimers, minibodies, diabodies, and multimers thereof and bispecific antibody fragments.

In another embodiment, the recombinant protein is an enzyme such as a therapeutic enzyme. In a specific embodiment, the therapeutic enzyme is butyrylcholinesterase. Butyrylcholinesterase (also known as pseudocholinesterase, plasma cholinesterase, BCHE, or BuChE) is a non-specific cholinesterase enzyme that hydrolyses many different choline esters. In humans, it is found primarily in the liver and is encoded by the BCHE gene. It is being developed as an antidote to organophosphate nerve-gas poisoning.

In yet another embodiment, the recombinant protein is a vaccine or a Virus-Like Particle (VLP) (for example, a VLP based on the M (membrane) protein of the Porcine Epidemic Diarrhea (PED) virus). The M protein is glycosylated (UTIGER et al. 1995).

In one embodiment, a signal peptide that directs the polypeptide to the secretory pathway of plant cells may be placed at the amino termini of recombinant proteins, including antibody HCs and/or LCs. In a specific embodiment, a peptide derived from Arabidopsis thaliana basic chitinase signal peptide (SP), for example MAKTNLFLFLIFSLLLSLSSA (SEQ ID NO:40), is placed at the amino-(N-) termini of both the HC and LC (Samac et al., 1990).

In another embodiment, the native human butyrylcholinesterase signal peptide (SP), namely MHSKVTIICIRFLFWFLLLCMLIGKSHT (SEQ ID NO:41), is placed at the amino-(N-) terminus of a therapeutic enzyme such as butyrylcholinesterase (GenBank: AAA99296.1).

Other signal peptides can be mined from GenBank [http://www.ncbi.nlm.nih.gov/genbank/] or other such databases, and their sequences added to the N-termini of the HC or LC, nucleotides sequences for these being optimized for plant preferred codons as described above and then synthesized. The functionality of a SP sequence can be predicted using online freeware such as the SignalP program [http://www.cbs.dtu.dk/services/SignalP/].

In a specific embodiment, the nucleic acid molecule encoding the recombinant protein is optimized for plant codon usage. In particular, the nucleic acid molecule can be modified to incorporate preferred plant codons. In a specific embodiment the nucleic acid molecule is optimized for expression in Nicotiana.

As used herein, the term “sequence identity” refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions multiplied by 100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul (1990), modified as in Karlin and Altschul (1993). Such an algorithm is incorporated into the NBLAST and XBLAST programs of Altschul et al. (1990). BLAST nucleotide searches can be performed with the NBLAST nucleotide program parameters set, e.g., for score=100, wordlength=12 to obtain nucleotide sequences homologous to a nucleic acid molecules of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al. (1997). Alternatively, PSI-BLAST can be used to perform an iterated search which detects distant relationships between molecules (Altschul et al., 1997). When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller (1988). Such an algorithm is incorporated in the ALIGN program (version 2.0) which is part of the Genetics Computer Group (GCG) sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

### Plants and Plant Cells

The disclosure also provides a plant or plant cell expressing a vector or T-DNA region or portion thereof as described herein. The expression is optionally stable or transient expression.

With respect to stable expression, as known in the art, T-DNA expressed from a vector may integrate into a plant genome at one, two or multiple sites. These sites are referred to herein as T-DNA insertion loci or T-DNA insertion sites. The nucleic acid sequence inserted at the T-DNA insertion locus is referred to as a “T-DNA insertion”. For example, the genome of the plant or plant cell described herein includes at least one T-DNA insertion. T-DNA insertions may comprise single, double or multiple insertions of various orientations.

In addition, the T-DNA insertions can be complete or incomplete. In a complete T-DNA insertion, the entire T-DNA region from the vector is inserted into the plant genome. In an incomplete insertion, only a portion of the T-DNA region from the plasmid is inserted into the plant genome (also known as a truncated T-DNA insertion). In one embodiment, the T-DNA insertion comprises or consists of the sequence between the LB and RB sequences. In another embodiment, the T-DNA insertion comprises or consists of the sequence between the LB and RB sequences plus 1-5 bp of the flanking LB and/or RB sequence. In another embodiment, the T-DNA insertion comprises or consists of most of the sequence between the LB and RB sequences; however, truncations of the T-DNA sequence from either end are possible.

The plant or plant cell may be heterozygous or homozygous for the T-DNA insertion. In other words, one or both copies of the genome of the plant or plant cell may contain the T-DNA insertion.

Also provided herein is a plant or plant cell that expresses an exogenous post-translational modification enzyme, wherein the coding sequence of the post-translation modification enzyme is integrated into the genome of the plant or plant cell and wherein the coding sequence of the post-translation modification enzyme has an engineered 5′ upstream sequence as described herein. Also provided is a plant or plant that expresses an exogenous post-translational modification enzyme, wherein the coding sequence of the post-translation modification enzyme is integrated into the genome of the plant or plant cell and wherein the coding sequence of the post-translation modification enzyme lacks an associated promoter sequence and/or a 5′ untranslated region (5′UTR) sequence. Further provided is a plant or plant that expresses an exogenous post-translational modification enzyme, wherein the coding sequence of the post-translation modification enzyme is integrated into the genome of the plant or plant cell and wherein the coding sequence of the post-translation modification enzyme has only a small fragment (for example, 3, 4, 5, 6, 7, 8, 9, 10, 15 or 20 contiguous base pairs) of a promoter sequence.

The plant or plant cell may be any plant or plant cell, including, without limitation, tobacco plants or plant cells, tomato plants or plant cells, maize plants or plant cells, alfalfa plants or plant cells, a Nicotiana species such as Nicotiana benthamiana or Nicotiana tabacum, rice plants or plant cells, Lemna major or Lemna minor (duckweeds), safflower plants or plant cells or any other plants or plant cells that are both agriculturally propagated and amenable to genetic modification for the expression of recombinant or foreign proteins.

In a specific embodiment of the present disclosure, the plant or plant cell is a tobacco plant. In another embodiment, the plant is a Nicotiana plant or plant cell, and more specifically a Nicotiana benthamiana or Nicotiana tabacum plant or plant cell. In another embodiment, the plant is an RNAi-based glycomodified plant. In another embodiment, the plant is a chemically mutagenized plant line, zinc-finger modified plant line or a CRISPR modified plant line. In a more specific embodiment the plant exhibits RNAi-induced gene-silencing of endogenous alpha-1,3-fucosyltransferase (FT) and beta-1,2-xylosyltransferase (XT) genes. In another embodiment, the plant or plant cell is a KDFX plant or plant cell as described for example in WO2018098572. In yet another embodiment, the plant or plant cell is a ΔXT/FT plant or plant cell (as published in Strasser et al., 2008). In yet another embodiment, the plant or plant cell is an N. benthamiana plant which has been selected from mutagenesis such that neither the FT and XT genes, nor the proteins encoded by the FT or XT genes are functional. For example, mutagenesis-based point mutations can result in early stop codons and therefore no protein expression, or true knock-outs (for example, those obtained using the CRISPR methodology) in which the promotor or coding region is excised and therefore there is no transcript produced. EMS (ethyl methane sulfonate) can also introduce point mutations, which could be screened for in such genes of interest.

As used herein, the term “plant” includes a plant cell and a plant part. The term “plant part” refers to any part of a plant including but not limited to the embryo, shoot, root, stem, seed, stipule, leaf, petal, flower bud, flower, ovule, bract, trichome, branch, petiole, internode, bark, pubescence, tiller, rhizome, frond, blade, ovule, pollen, stamen, and the like.

As described herein, in addition to the post-translational modification enzyme, in one embodiment, the T-DNA region further comprises a sequence that encodes another recombinant protein, which can be expressed in and isolated from a plant or plant cell. In other embodiments, a second nucleic acid molecule that encodes a recombinant protein is expressed from a separate vector in the plant or plant cell.

In one embodiment, the plant or plant cell is further modified to increase expression of the recombinant protein.

For example, in one embodiment, the plant or plant cell optionally also expresses the P19 protein from Tomato Bushy Stunt Virus (TBSV; Genbank accession: M21958). In a preferred embodiment, the P19 protein from TBSV is expressed from a nucleic acid molecule which has been modified to optimize expression levels in Nicotiana plants. In a specific embodiment, the modified P19-encoding nucleic acid molecule has the sequence shown in SEQ ID NO:29.

The P19 protein can be expressed from an expression vector comprising a single expression cassette or from an expression vector containing one or more additional cassettes, wherein the one or more additional cassettes comprise transgenic DNA encoding one or more recombinant proteins or RNA-interference inducing hairpins.

In another embodiment, the plant or plant cell has reduced expression of endogenous ARGONAUTE proteins, for example ARGONAUTE1 (AGO1) and ARGONAUTE4 (AGO4). The expression of endogenous ARGONAUTE proteins can be reduced by any method known in the art, including, but not limited to, RNA interference techniques.

Other methods of increasing expression of the recombinant protein in the plant or plant cell are also known in the art. These methods include, but are not limited to the use of plant virus based expression systems such as Gemini virus vectors (MoR et al. 2003), yellow bean dwarf virus (HuANG et al. 2010), cowpea mosaic virus (e.g., pEAQ vectors) (SAINSBURY et al. 2009) and Tobacco mosaic virus vectors (e.g., Magnifection® vectors) (GLEBA et al. 2005) or the use of other viral silencing suppressor proteins such as V2 (NAIM et al. 2012). It has also been shown that incorporating chimeric 3′ flanking regions can enhance expression (DIAMOS AND MASON 2018).

### Methods

The inventors have demonstrated that the expression and glycosylation patterns of recombinant proteins produced in plants can be modified by reducing the expression of enzymes that confer post-translational modification activities through the use of the plant expression vectors described herein.

Accordingly, the disclosure provides a method of optimizing the expression and/or glycosylation pattern of a recombinant protein produced in a plant or plant cell comprising:

(a) introducing into the plant or plant cell a T-DNA vector as described herein,

(b) introducing into the plant or plant cell a nucleic acid molecule encoding a recombinant protein into the plant or plant cell; and

(c) growing the plant or plant cell to obtain a plant that expresses the recombinant protein.

In one embodiment, the disclosure provides method of optimizing expression of a recombinant protein produced in a plant or plant cell, the method comprising:

(a) introducing into the plant or plant cell a T-DNA vector as described herein,

(b) introducing a second nucleic acid molecule encoding the recombinant protein into the plant or plant cell, and

(c) growing the plant or plant cell to obtain a plant that expresses the recombinant protein.

In one embodiment, the recombinant protein has increased expression compared to the expression of the recombinant protein produced in a control plant or plant cell.

As used herein, the term “increased expression” refers to at least 10%, 20%, 30%, 40%, 50%, 60%, 70%, 80%, 90%, 100% or more than 100% increased expression over expression of the recombinant protein in a control plant or plant cell. Numerous methods of measuring protein expression are known in the art.

In one embodiment, a “control plant or plant cell” is a plant or plant cell where the post-translational modification enzyme is expressed behind a strong or intermediate strength promoter, for example the double enhancer 35S promoter, 35S promoter, Act2 promoter or Act8 promoter. In another embodiment, a “control plant or plant cell” is a plant or plant cell with the same genetic background as the plant or plant cell into which the T DNA vector is introduced. In one embodiment, the control plant or plant cell is a wild-type plant or plant cell. In another embodiment, the control plant or plant cell is genetically engineered for knock-out or knock-down of beta-1,2-xylosyltransferase and/or alpha-1,3-fucosyltransferase activities (e.g., KDFX as described in WO2018098572 or ΔXT/FT as published in Strasser et al., 2008).

The disclosure also provides a method of increasing the amount of galactosylation on a recombinant protein produced in a plant or plant cell, the method comprising:

(a) introducing into the plant or plant cell a plant T-DNA vector as described herein,

(b) introducing a second nucleic acid molecule encoding the recombinant protein into the plant or plant cell, and

(c) growing the plant or plant cell to obtain a plant that expresses the recombinant protein, and wherein the post-translational modification enzyme is GaIT.

In one embodiment, the recombinant protein produced in the plant or plant cell has a higher amount of galactosylation compared to the recombinant protein produced in a control plant or plant cell. Optionally, recombinant protein produced in the plant or plant cell has at least 10%, 20%, 30%, 40%, 50%, 60%, 70%, 80%, 90% or 100% more galactosylation compared to recombinant protein produced in a control plant or plant cell. In another embodiment, the recombinant protein produced in the plant or plant cell has at least 10%, 20%, 30%, 40%, 50%, 60%, 70%, 80%, 90% or 100% galactosylation. The amount of galactosylation is optionally measured as a percentage of glycan species which contain galactose. Numerous methods of measuring galactosylation levels are known in the art. For example, galactosylation can be measured by using HPLC or MS methods.

The disclosure also provides a method of increasing the amount of AGn and/or AA glycans or the amount of AGn glycans over AA glycans on a recombinant protein produced in a plant or plant cell, the method comprising:

(a) introducing into the plant or plant cell a T-DNA vector as described herein,

(b) introducing a second nucleic acid molecule encoding the recombinant protein into the plant or plant cell, and

(c) growing the plant or plant cell to obtain a plant that expresses the recombinant protein,

and wherein the post-translational modification enzyme is GaIT.

In one embodiment, the recombinant protein produced in the plant or plant cell has a higher amount of AGn and/or AA glycans compared to the recombinant protein produced in a control plant or plant cell. Optionally, recombinant protein produced in the plant or plant cell has at least 10%, 20%, 30%, 40%, 50%, 60%, 70%, 80%, 90% or 100% more AGn and/or AA glycans compared to recombinant protein produced in a control plant or plant cell. In another embodiment, the recombinant protein produced in the plant or plant cell has at least 10%, 20%, 30%, 40%, 50%, 60%, 70%, 80%, 90% or 100% AGn and/or AA glycans.

In another embodiment, the recombinant protein produced in the plant or plant cell has a greater amount of AGn glycans over AA glycans compared to the recombinant protein produced in a control plant or plant cell.

The amount of AGn and/or AA glycans are optionally measured as an absolute value or as a percentage of totally glycan species. Numerous methods of measuring AGn and AA glycan content are known in the art. For example, AGn and AA glycan content can be measured by using HPLC or MS methods.

The disclosure also provides a method of increasing the amount of alpha-1,6-fucosylated glycans on a recombinant protein produced in a plant or plant cell, the method comprising:

(a) introducing into the plant or plant cell the plant a T-DNA vector as described herein,

(b) introducing a second nucleic acid molecule encoding the recombinant protein into the plant or plant cell, and

(c) growing the plant or plant cell to obtain a plant that expresses the recombinant protein,

and wherein the post-translational modification enzyme is FucT, optionally an alpha-1,6-FucT.

In one embodiment, the recombinant protein produced in the plant or plant cell has a higher amount of alpha-1,6-fucosylated glycans compared to the recombinant protein produced in a control plant or plant cell. The amount of alpha-1,6-fucosylated glycans are optionally measured as an absolute value or as a percentage of totally glycan species. Optionally, recombinant protein produced in the plant or plant cell has at least 10%, 20%, 30%, 40%, 50%, 60%, 70%, 80%, 90% or 100% more alpha-1,6-fucosylated glycans compared to recombinant protein produced in a control plant or plant cell. In another embodiment, the recombinant protein produced in the plant or plant cell has at least 10%, 20%, 30%, 40%, 50%, 60%, 70%, 80%, 90% or 100% alpha-1,6-fucosylated glycans. Numerous methods of measuring alpha-1,6-fucosylated glycan content are known in the art. For example, alpha-1,6-fucosylated glycans can be measured by using HPLC or MS methods.

The disclosure also provides a method of decreasing the proportion of aglycosylation on recombinant protein produced in a plant or plant cell, the method comprising:

(a) introducing into the plant or plant cell a T-DNA vector as described herein,

(b) introducing a second nucleic acid molecule encoding the recombinant protein into the plant or plant cell, and

(c) growing the plant or plant cell to obtain a plant that expresses the recombinant protein, and wherein the post-translational modification enzyme is STT3D.

In one embodiment, recombinant protein has a lower proportion of aglycosylated protein, optionally compared to the recombinant protein produced in a control plant or plant cell. In one embodiment, the proportion of aglycosylated protein is at least 10%, 20%, 30%, 40%, 50%, 60%, 70%, 80%, 90% or 100% lower compared to the proportion of aglycosylated protein produced in a control plant or plant cell.

Glycosylation site occupancy of glycoproteins can be calculated, for example, by quantification of bands from immunoblots, as an aglycosylated polypeptide will migrate quicker during electrophoresis than the glycosylated peptide; however, this can be difficult to estimate as electrophoretic separations can be quite small. Another method is to use MS-based quantification of peptides from purified proteins. Both of these methods are used in the following publication: “Castilho, A., G. Beihammer, C. Pfeiffer, K. Goritzer, L. Montero-Morales et al., 2018. An oligosaccharyltransferase from Leishmania major increases the N-glycan occupancy on recombinant glycoproteins produced in Nicotiana benthamiana. Plant Biotechnol J. 6: 1700-1709.”

In another example, measurement for the amount of glycosylation site occupancy (and, the lack thereof for aglycosylation assessment) for an antibody involves purifying the recombinant protein, such as by using the Ab SpinTrap (GE Healthcare), followed by dialysis against PBS overnight at 4° C.; weak cation exchange high performance liquid chromatography (WCX-HPLC) is then performed to determine the proportion of glycosylated, hemi-glycosylated, and aglycosylated antibody. This is done by injection of antibody sample into an Agilent Bio Mab, NPS, SS column (4.6×250 mm, 5 μm, P/N 5190-2405; Agilent). Agilent ChemStation software is then used to calculate the peak areas of the resultant peaks; fractional peak areas divided by total peak areas are then calculated to determine percentage of glycosylation site occupancy.

The disclosure also provides a method of increasing the amount of AAF and AGnF glycans (by virtue of alpha-1,6-linkages to the fucose moiety) and reducing the amount of AA and AGn glycans on recombinant protein produced in a plant or plant cell, the method comprising:

(a) introducing into the plant or plant cell introducing into the plant or plant cell a T-DNA vector as described herein, wherein the T-DNA vector comprises both an alpha-1,6-FucT and a GaIT, wherein of at least one of the enzymes is downstream of a non-traditional promoter sequence as described herein,

(b) introducing a second nucleic acid molecule encoding the recombinant protein into the plant or plant cell, and

(c) growing the plant or plant cell to obtain a plant that expresses the recombinant protein.

In one embodiment, the recombinant protein produced in the plant or plant cell has a higher amount of AAF and AGnF glycans compared to the recombinant protein produced in a control plant or plant cell. The amount of AAF and/or AGnF glycans are optionally measured as an absolute value or as a percentage of totally glycan species. Optionally, recombinant protein produced in the plant or plant cell has at least 10%, 20%, 30%, 40%, 50%, 60%, 70%, 80%, 90% or 100% more AAF and/or AGnF glycans compared to recombinant protein produced in a control plant or plant cell. In another embodiment, the recombinant protein produced in the plant or plant cell has at least 10%, 20%, 30%, 40%, 50%, 60%, 70%, 80%, 90% or 100% AAF and/or AGnF glycans. Numerous methods of measuring AAF and AGnF glycan content are known in the art. For example, AAF and AGnF glycan content can be measured by using HPLC or MS methods.

The phrase “introducing” a vector or a nucleic acid molecule into a plant or plant cell includes both the stable integration of the nucleic acid molecule into the genome of a plant cell to prepare a transgenic plant as well as the transient integration of the nucleic acid into a plant or part thereof.

The nucleic acid molecules and vectors may be introduced into the plant cell using techniques known in the art including, without limitation, vacuum infiltration, electroporation, an accelerated particle delivery method, a cell fusion method or by any other method to deliver the expression vectors to a plant cell, including Agrobacterium mediated delivery, or other bacterial delivery such as Rhizobium sp. NGR234, Sinorhizobium meliloti and Mesorhizobium loti (Chung et al, 2006).

The plant cell may be any plant cell, including, without limitation, tobacco plants, tomato plants, maize plants, alfalfa plants, Nicotiana benthamiana, Nicotiana tabacum, Nicotiana tabacum of the cultivar cv. Little Crittenden, rice plants, Lemna major or Lemna minor (duckweeds), safflower plants or any other plants that are both agriculturally propagated and amenable to genetic modification for the expression of recombinant or foreign proteins.

In one embodiment, nucleic acid molecules and expression vectors are introduced in a RNAi-based glycomodified plant. In a specific embodiment, the plant is an N. benthamiana plant. In a more specific embodiment the N. benthamiana plant exhibits RNAi-induced gene-silencing of endogenous fucosyltransferase (FT) and xylosyltransferase (XT) genes. In another embodiment, the plant or plant cell is a KDFX plant or plant cell as described for example in WO2018098572. In another embodiment, the plant or plant cell is a ΔXT/FT plant (as published in Strasser et al., 2008). In yet another embodiment, the plant or plant cell is an N. benthamiana plant which has been mutagenized so as to have complete knockouts of all FT and XT gene functions.

The phrase “growing a plant or plant cell to obtain a plant that expresses a recombinant protein” includes both growing transgenic plant cells into a mature plant as well as growing or culturing a mature plant that has received the nucleic acid molecules encoding the recombinant protein. One of skill in the art can readily determine the appropriate growth conditions in each case.

In another embodiment, stable transgenic plants are made. Methods of making stable transgenic plants can include, for example, the steps of (a) introducing the T-DNA vector into a bacterial species capable of introducing DNA to plants for transformation, (b) transforming cells of the plant with the bacteria containing the T-DNA vector, (c) culturing cells to grow to whole plants, and (d) selection of transformed plants. After selection of PTM enzyme-expressing primary transgenic plants, or concurrent with selection of antibody-expressing plants, derivation of homozygous stable transgenic plant lines can be performed. For example, primary transgenic plants maybe grown to maturity, allowed to self-pollinate, and produce seed. Homozygosity can be verified by the observation of 100% resistance of seedlings on solid agar media containing the appropriate drug used to select for the development of primary plants. A transgenic line with single T-DNA insertions, that are shown by molecular analysis to produce most amounts of PTM enzyme, can be chosen for breeding to homozygosity and seed production, ensuring subsequent sources of seed for homogeneous production of antibody by the stable transgenic or genetically modified crop (Olea-Popelka et al., 2005; McLean et al., 2007; Yu et al., 2008).

The following non-limiting Examples are illustrative of the present disclosure:

### Example 1

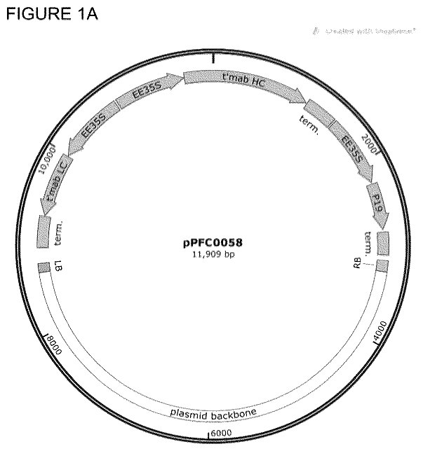

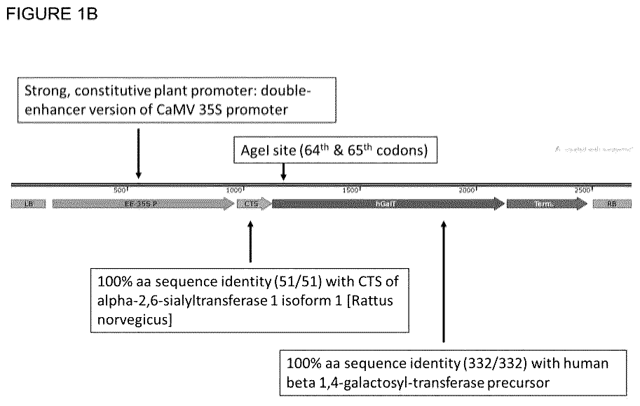

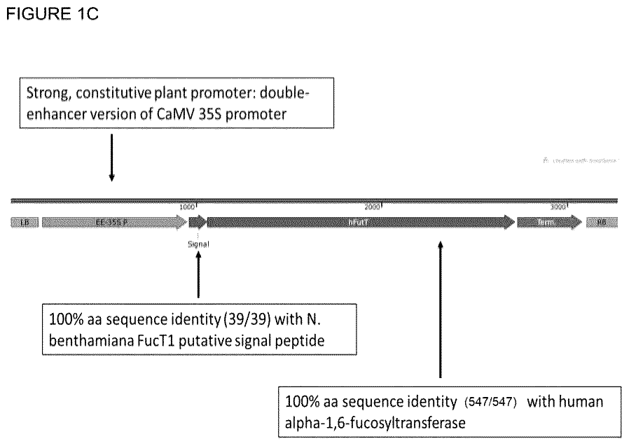

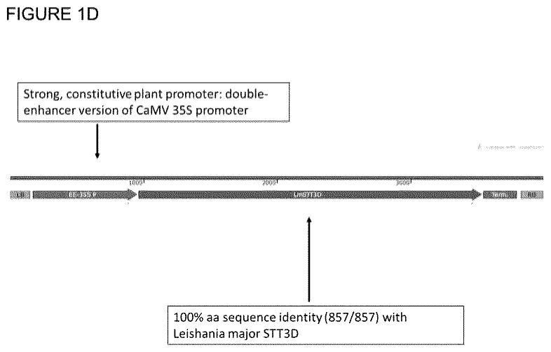

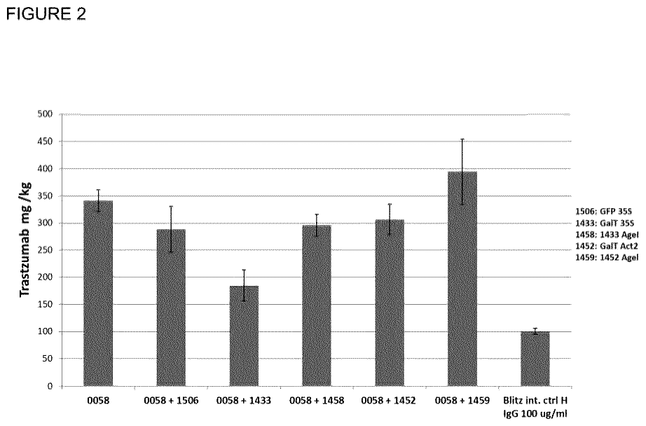

Transient expression of recombinant proteins such as antibodies in plants typically involves Agroinfiltration to introduce antibody heavy chain (HC) and light chain (LC) polypeptide genes into plant cells. Introduction of other genes such as for the tombusvirus P19 RNA silencing suppressor can also be performed, to enhance transient expression of recombinant proteins in plants. Introduction of yet other genes such as those that encode enzymes which post-translationally modify (PTM) transiently expressed recombinant proteins can also be performed; for example, this can be performed to control post-translational modifications of recombinant proteins, such as glycosylation. In the first example, an attempt was made to co-express a chimeric human beta-1,4-galactosyltransferase (hGaIT) under the control of a strong promoter (i.e., double-enhancer version of CaMV 35S). A vivoXPRESS® expression vector containing genes for the HC and LC of trastuzumab antibody plus P19, PFC0058, was introduced by Agroinfiltration into Nicotiana benthamiana plant cells: alone; and with five other individual vectors. Four of these six vectors are shown in FIG. 1. FIG. 2 shows the amounts of trastuzumab that were measured for those six treatments, in mg antibody per kg plant fresh weight, along with error bars indicating standard error of the mean (SEM) for each treatment. Trastuzumab was expressed from vector PFC0058 at approximately 350 mg/kg. Trastuzumab was expressed equivalently to the PFC0058 vector alone treatment in four other treatments involving four other vectors, as seen for results in which the SEM error bars overlapped. One treatment that resulted in statistically equivalent expression to PFC0058 alone involved co-expression with vector PFC1506, containing a double enhancer 35S promoter (EE35S) driving transcription of Green Fluorescent Protein (GFP) coding sequence; this result was not surprising as it showed that plant cells can co-express more than one recombinant protein using the same promoter system, and that the second recombinant protein (in this case, GFP) does not affect the amount of recombinant antibody that is expressed. It was surprising that strong expression of chimeric hGaIT enzyme on vector PFC1433 containing the EE35S promoter (see FIG. 1B), caused statistically significant reduction of trastuzumab expression. Strong expression of hGaIT transcript was not likely responsible for this, as the treatment involving vector PFC1458, containing a frameshift mutation at a unique Agel site in the hGaIT coding sequence, resulted in statistically equivalent trastuzumab expression to the PFC0058 alone treatment. Also, expression of hGaIT from vector PFC1452, containing the relatively weaker Act2 promoter, also resulted in statistically equivalent trastuzumab expression to the PFC0058 alone treatment.

### Example 2

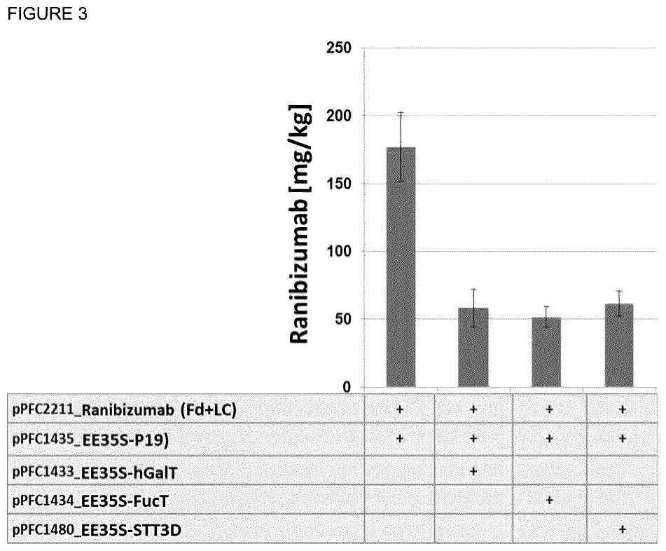

The experiment shown in FIG. 2 shows that strong expression of functional hGaIT enzyme from the EE35S promoter causes a reduction of antibody expression in plants. This was repeated with other antibodies and the same result was found (data not shown). Without being bound by theory, it was hypothesized that this was due to the post-translational glycosylation of recombinant antibodies in plants. This was tested by expressing another recombinant antibody, i.e., ranibizumab, which is a Fab-type antibody that lacks heavy chain CH2 and CH3 components; thus, it consists of a LC and a Fd chain. Because Fabs lack the CH2 N-linked glycosylation site, ranibizumab is not glycosylated. Vector PFC2211 (schematic not shown), containing coding sequences for the ranibizumab LC and Fd polypeptides both driven by the EE35S promoter, and vector PFC1435, containing P19 driven by the EE35S promoter were expressed together, and with three other single-gene vectors as shown in FIG. 3. While the Fab-type antibody is not glycosylated, strong expression of three different PTM/glycomodification enzymes (i.e., hGaIT, FucT and STT3D), all driven by the EE35S promoter, caused severe reduction of ranibizumab expression. Thus, without being bound by theory, it is believed that strong expression of PTM enzymes causes reduction of expression of antibodies in plants not solely because of their glycosylation activities but by some other mechanism or mechanisms, which need not be the same for all PTM enzymes.

### Example 3

The use of vectors containing strong promoters driving expression of post-translational modification enzymes in plant-based protein production methods is therefore at times ineffective, because resulting transient expression processes and resulting stable transgenic plants typically produce lesser amounts of recombinant therapeutic protein; also, glycoproteins are produced with overly complex mixtures of glycans that also contain significant amounts of incompletely processed glycans (KALLOLIMATH et al. 2017). Furthermore, upwards of 20% of target proteins typically lack glycosylation (i.e., upwards of 20% aglycosylation).

In addition, stable transgenic plants expressing such promoter-plus vectors typically lose their post-translational modification activities when attempting to develop homozygous (or genetically homogeneous) lines by plant breeding. Without being bound by theory, it is believed that this occurs because stable transgenic plants cannot likely tolerate strong expression of these genes and therefore offspring plants from breeding programs impose transgene-silencing mechanisms so as to remain viable. The vectors described below were designed to overcome some of these problems.

### Methods

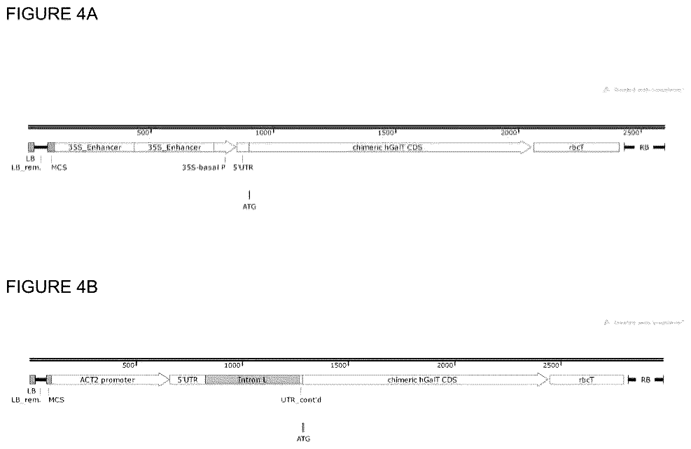

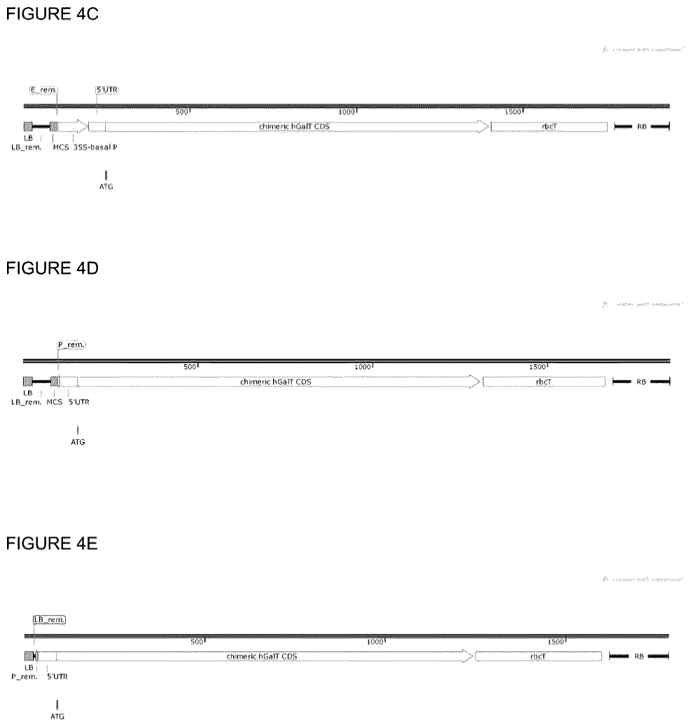

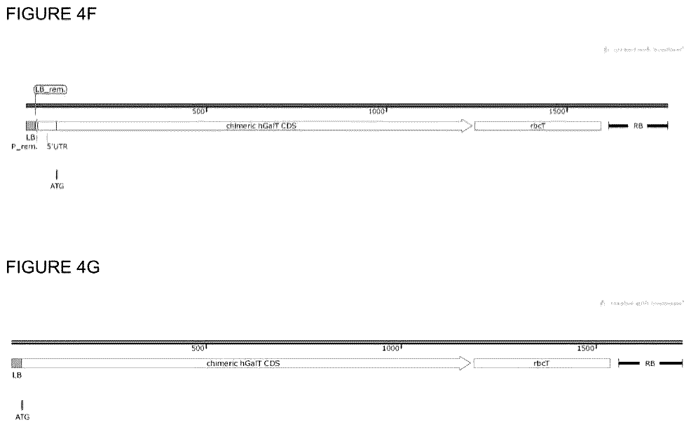

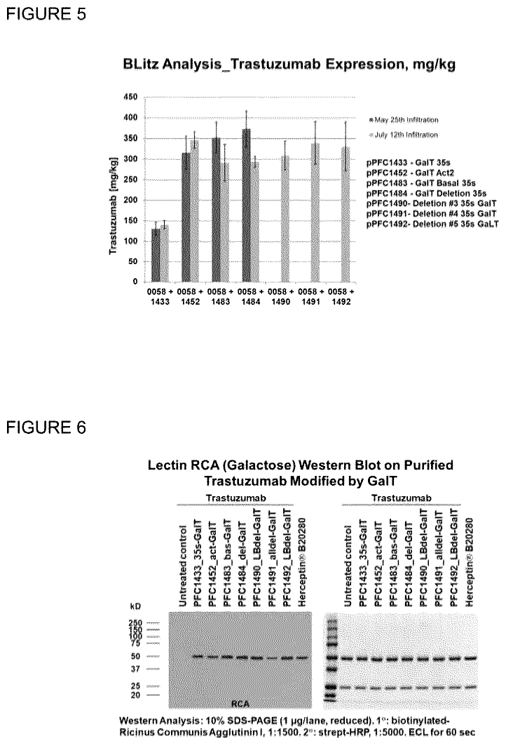

Seven GaIT expression plasmids were constructed as vivoXPRESS® T-DNA vectors, containing either a double enhancer version of the CaMV 35S promoter or deletions thereof, or the Arabidopsis Actin2 gene promoter (A_{N} et al. 1996). First, pPFC1433 was constructed, consisting (directionally) of the minimal 25-bp Agrobacterium tumefaciens T-DNA LB repeat; 53-bp more Agrobacterium DNA from the 3′ side of the 25-bp repeat, as found in pBIN19 (BEVAN 1984); 4 restriction endonuclease recognition sequences; the double-enhancer version of the CaMV 35S promoter; a 51-bp 5′ UTR, including a plant Kozak box for start of translation. Oligonucleotide mediated mutagenesis was performed to derive 5 promoter and/or UTR deletion mutants of pPFC1433: (i) pPFC1483, a basal promoter version of the 35S promoter, lacking both enhancers; (ii) pPFC1484, a near-complete promoter deletion, leaving only 6 bp of basal promoter; (iii) pPFC1490, the same 6-bp near-complete promoter deletion, but with a second deletion of restriction sites plus 46 bp from downstream of the 3′ side of the 25-bp LB repeat; (iv) pPFC1492, a mere 5-bp deletion of pPFC1490, again from the 3′ side of the 25 bp repeat; (v) pPFC1491, a complete deletion of all promoter, UTR and other genetic elements, placing the ATG start of translation codon for GaIT directly adjacent to the 3′ side of the minimal 25-bp LB repeat. The 7^{th} plasmid, pPFC1452, containing the Arabidopsis thaliana ACT2 gene promoter driving GaIT transcription, was constructed independently. FIG. 4 and Tables 1 and 10 below describe these GaIT expression vectors.

TABLE 1
Description of promoters and associated genetic elements driving
transcription of GaIT coding sequence on vectors described within.

| | Agrobacterium DNA | | | | | |
|---|---|---|---|---|---|---|
| | LB | Sequence 3’ | | | 5’ UTR | ATG |
| | 25 bp | of 25-bp | Restriction | | (51 bp, incl. | (translation |
| PFC # | repeat | LB (53 nt) | sites | Promoter | Kozak box) | start codon) |
| 1433 | Yes¹ | Yes² | 4⁴ | Double | 51 bp | ATG |
| | | | | enhancer | UTR¹² | |
| | | | | 35S⁸ | | |
| | | | | PLUS Basal | | |
| | | | | 35S⁹ | | |
| 1483 | Yes¹ | Yes² | 3⁵ | Basal 35S⁹ | 51 bp¹² | ATG |
| 1484 | Yes¹ | Yes² | 3⁵ | Only 6 nt from | 51 bp¹² | ATG |
| | | | | 3’ end¹⁰ | | |
| 1490 | Yes¹ | Deletion of 46 | None | Only 6 nt from | 51 bp¹² | ATG |
| | | bp from 3’ end³ | | 3’ end¹⁰ | | |
| 1492 | Yes¹ | Complete 53- | None; 2 nt | Only 6 nt from | 51 bp¹² | ATG |
| | | bp deletion | cloning | 3’ end¹⁰ | | |
| | | | artefact6 | | | |
| 1491 | Yes¹ | Complete 53- | None | None | None | ATG |
| | | bp deletion | | | | |

| | | | | | |
|---|---|---|---|---|---|
| 1452 | Yes¹ | Yes² | 3⁷ | A.thal. Act2, incl. own | ATG |
| | | | | UTR; same Kozak box | |
| | | | | as others¹¹ | |

¹SEQ ID NO: 23
²SEQ ID No: 30
³SEQ ID NO: 31
⁴SEQ ID NO: 32
⁵SEQ ID NO: 33
⁶SEQ ID NO: 34
⁷SEQ ID NO: 35
⁸SEQ ID NO: 36
⁹SEQ ID NO: 37
¹⁰SEQ ID NO: 38
¹¹1183-nt sequence (AN et al. 1996)
¹²SEQ ID NO: 39

Each of the GaIT expression plasmids were introduced into Agrobacterium tumefaciens strain EHA105 (HOOD et al. 1993), grown as shake flask cultures and used for vacuum infiltration of Nicotiana benthamiana plants for transient expression. Each of these plasmids were individually vacuum infiltrated with a 3-gene T-DNA expression vector containing the P19 gene and 2 genes encoding the heavy chain (HC) and light chain (LC) of trastuzumab; all 3 genes are driven by their own double-enhancer version of the CaMV35S promoter. General methods required for these techniques are available in (GARABAGI et al. 2012a; GARABAGI et al. 2012b). A reference for the expression of trastuzumab, using another vector system, is (GRos et al. 2010).

Trastuzumab antibody was expressed from the 3-gene T-DNA expression vector with simultaneous expression of hGaIT from one of the seven vectors described above. Each treatment involved co-infiltration of N. benthamiana plants with two Agrobacterium strains: the 3-gene T-DNA expression vector and one hGaIT vector, each at an OD₆₀₀ of 0.2 according to published methods (GARABAGI et al. 2012a; GARABAGI et al. 2012b). Green leaf biomass was harvested (excluding leaf midribs) 7 days post infiltration (dpi). Trastuzumab amounts were measured using Pall:ForteBio BLItz instrumentation (https://www.fortebio.com/blitz.html), and expression is reported as mg trastuzumab/kg green biomass. Four biological replicates were performed for each treatment, and standard errors are presented on each histogram bar.

Trastuzumab was purified using one step Protein G affinity purification method (Ab SpinTrap, GE Healthcare, cat #28-4083-47). In brief, total soluble plant protein extract was incubated with protein G-coated beads, and incubated at 4 C for 2.5 hr. Antibody captured beads were reloaded into the column and washed with four times with Tris-buffered saline, antibody was then eluted with 0.1 M glycine at pH 2.7 and neutralized with Tris buffered. Purified antibody was further dialyzed against PBS. For Coomassie blue gel staining, equivalent (4 μg) amounts of antibody were separated on 10% SDS-PAGE under reduced and non-reduced conditions. For immunoblot analysis, equivalent (1 μg) amounts of antibody were applied to 10% SDS-PAGE gels under reduced condition. Gels were used for electro-transfer of proteins to PVDF membrane (GE Healthcare), and probed with biotinylated Ricinus communis Agglutinin I (Vector Labs), followed by streptavidin-conjugated HRP (BioLegend). Signal development was revealed using SuperSignal West Pico Chemiluminescent Substtrate (ThermoFisher). For the quantification and analysis of glycan species, the rationale we used were previously some glycan species have been compared and identified by both Mass Spectoscopy and Hydrophilic-Interaction Liquid Chromatography (HILIC) using TSKgel Amide-80 column (Tosoh Bioscience) via UFLC methods. Therefore, the relative retention time for the glycan species under HILIC UFLC analysis will be used for identification. Autointegration method was used to calculate the quantity of each glycan species peak. Glycan was prepared by using GlykoPrep Rapid N-Glycan Preparation kit (Prozyme).

### Results:

FIG. 6 shows trastuzumab antibody expression 7 days post infiltration (dpi) with and each of the 7 hGaIT vectors. As can be seen, antibody expression with pPFC1433 is less than half the antibody expression with the 6 other vectors (i.e., <150 mg/kg cf. ˜300 mg/kg or greater).

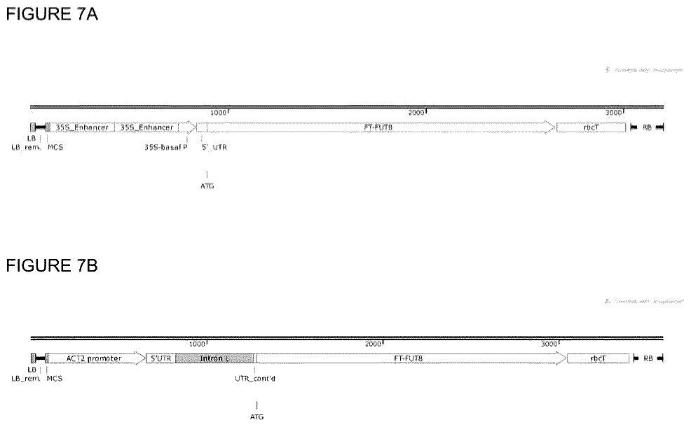

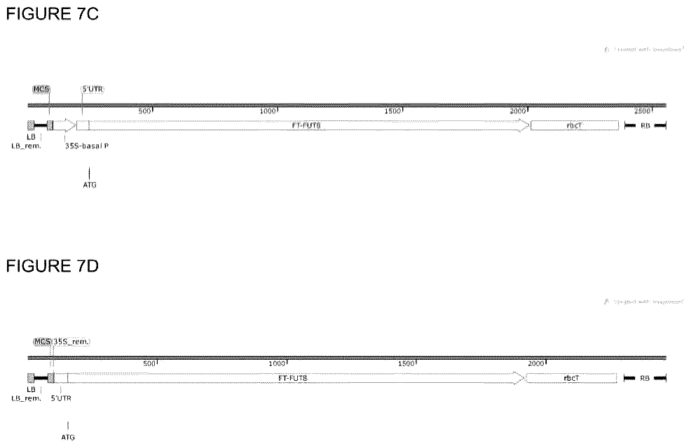

FIG. 7 shows a side-by-side comparison of a Coomassie blue-stained SDS-PAGE gel (confirming equivalent loadings) and a Western blot probed with galactose-specific RCA lectin. On the Western blot, the intensity of signal increases from vector 1433 (double enhancer 35S promoter driving hGaIT expression), to vector 1452 (Act2 promoter driving hGaIT), to vectors 1483 (basal 35S promoter), 1484 (35S promoter deletion but with 5′ UTR), 1490 (35S promoter and LB flanking deletions, but with 5′ UTR) and 1492 (35S more complete promoter and LB flanking deletions, but with 5′ UTR). RCA signal intensity is significantly reduced with co-expression of pPFC1491 (complete deletions of promoter, LB flanking sequence and 5′ UTR), but is still detected.

Table 3 shows abundance of glycan species measured on trastuzumab antibody samples from co-expression with 6 hGaIT vectors; sample from treatment with vector 1492 was not included due to degree of similarity with vector 1490 (these 2 vectors differ by only 5 nucleotides upstream of the 5′ UTR). (Trastuzumab expression from the 3-gene T-DNA expression vector alone, i.e., without a hGaIT vector, was also performed. As expected, trastuzumab expression alone resulted in predominantly GnGn glycans, i.e., 88.5%, with 6 other measurable glycan species accounting for the remainder.) The strong EE35S promoter driving hGaIT on vector 1433 resulted in 12 measurable glycan species, with the 2 most abundant species being Man5Gn+/−Hex; these are hybrid-type glycans (between high mannose glycans and complex glycans), each of which occurs rarely on therapeutic antibodies (McLEAN 2017). Vector 1433 also resulted in relatively high amounts of GnM and high mannose (especially Man5) glycans. 1433 resulted in low amounts of galactosylated glycans, especially for AGn (1.8%) and AA (3.4%). The Act2 (1452) and basal 35S (1483) promoters resulted in similar types and abundances of glycan species, with especially high amounts of Man4Gn/AM, Man5Gn and GnM species; as with 1433, galactose species abundances are also low, although the AA species amounts are somewhat higher than for 1433. Vectors 1484 and 1490, both near-complete promoter deletions but both with the complete 5′ UTR, resulted in relatively high amounts of GnGn and galactosylated species; AGn and AA glycan species are similar in abundance, all being above 20% for both vectors. Vector 1491, having all genetic elements 5′ of the ATG start of translation deleted such that the ATG codon is directly adjacent the functional 25-nt LB sequence, results in a significant return to GnGn glycans (>50%). Vector 1491 also results in AGn glycans are greater than 20% while AA glycans are less abundant (6%). This is significant, as therapeutic antibody glycans such as those found on Herceptin® and Humira® also have a greater abundance of AGn and/or AGnF glycans over AA and/or AAF glycans, respectively (Table 2).

TABLE 2
Glycan content of Herceptin ® and Humira ®.

| | | Humira ® | |
|---|---|---|---|
| | Herceptin ® (avg. ± | (PlantForm | Humira ® (avg. ± |
| Glycoforms of | s.d.; Damen et al., | GlykoPrep | s.d.; Tebbey and |
| HC (%) | 2007)¹ | measurement)² | Declerck, 2016)³ |
| AGn⁴ or GnA | | 6.7 | |
| AGnF or GnAF | 39.7 ± 3.7 | 16.9 | 18.45 ± 1.80 |
| AAF | 9.5 ± 3.1 | | |
| AA | | 2.9 | |

¹Damen, C. W., W. Chen, A. B. Chakraborty, M. van Oosterhout, J. R. Mazzeo et al., 2009 Electrospray ionization quadrupole ion-mobility time-of-flight mass spectrometry as a tool to distinguish the lot-to-lot heterogeneity in N-glycosylation profile of the therapeutic monoclonal antibody trastuzumab. J Am Soc Mass Spectrom 20: 2021-2033.
In this paper, ESI-Q-IM-TOF-MS was performed on four different lots of Herceptin ® to determine lot-to-lot heterogeneity of this commercial antibody; refer to methodology within this paper for details.
²Results of single glycan measurement of Humira ® by PlantForm scientists (unpublished) using GlykoPrep ® analysis. Methods were according to the manufacturer. Briefly, glycans were released from antibody using PNGaseF and labeled with 2-AB (2-aminobenzamide) fluorescent dye according to GlykoPrep ® Rapid N-Glycan Preparation kit (PROzyme cat. no. GP24NG-LB). Labeled glycans were separated by Hydrophilic-Interaction Liquid Chromatography (HILIC) using a TSKgel Amide-80 column (Tosoh Bioscience) and identified by relative retention time for known glycan species. Autointegration was used to calculate the quantity of each glycan species peak.
Data from these measurements serve to clarify pooled glycan measurements for Humira ® given in the rightmost column.
³Tebbey, P. W., and P. J. Declerck, 2016 Importance of manufacturing consistency of the glycosylated monoclonal antibody adalimumab (Humira ®) and potential impact on the clinical use of biosimilars. Generics and Biosimilars Initiative Journal 5: 70-73.
This paper summarizes the results of glycan analyses of 381 batches of Humira ® produced between 2001 and 2013; some glycoforms are pooled (MGnF or GnMF and GnGnF; AGnF or GnAF and AAF; M5-M9) as a result of summarizing 381 data sets for Table 1 of this paper.
⁴Glycan structures can be viewed at http://www.proglycan.com/upload/IgG_Table_Rosetta.pdf

TABLE 3
Percentages of galactosylated and non-galactosylated species from above experimental samples.

| hGaIT vector | PFC1433 | PFC1452 | PFC1483 | PFC1484 | PFC1490 | PFC1491 |
|---|---|---|---|---|---|---|
| Short form | EE35S-GaIT | Act2-GaIT | BasaI35S-GaIT | LB+/UTR-GaIT | LB-UTR-GaIT | LB-GaIT |
| AGn | 1.8 | 2.4 | 2.3 | 20.5 | 20.9 | 21.3 |
| AA | 3.4 | 7.4 | 9.9 | 23.1 | 22.6 | 6.0 |
| Other | 39.2 | 44.0 | 49.8 | 17.0 | 16.9 | 7.7 |
| Galalctosylated | | | | | | |
| species* | | | | | | |
| Other Non- | 55.0 | 46.0 | 37.9 | 39.4 | 39.6 | 65.0 |
| Galalctosylated species** | | | | | | |
| TOTAL | 99.4 | 99.8 | 99.9 | 100 | 100 | 100 |

*Man4Gn/AM plus Man5Gn + Hex
**MM plus GnM plus GnGn plus Man5 plus Man5Gn plus M7 plus M8 plus M9

### DISCUSSION

Only the strongest promoter driving hGaIT expression resulted in reduced co-expression of trastuzumab, i.e., on vector PFC1433. This promoter, EE35S, also gave rise to significant amounts of high mannose and hybrid-type glycans as well as low amounts of galactosylated glycans (specifically, AA and AGn species). Without being bound by theory, this is considered to be due to overactivity of the galactosyltransferase and creation of inappropriately galactosylated glycans which fail to progress through to completion of the glycosylation pathway and create blockage in transit of precursor species via mechanisms such as competitive inhibition for enzyme substrate sites. Reduction of promoter strength on hGaIT resulted in lesser amounts of high mannose glycans; also, as promoter strength was further reduced, lesser amounts of hybrid glycans were produced. Only when the complete promoter and the complete 5′ UTR were removed, i.e., for the 1491 vector, did resulting glycans become less complex. Also, the ratio of AA to AGn glycans was significantly reduced with this vector. This may be important for pharmaceutical scientists attempting to develop procedures for expression of antibody therapeutics, as antibody therapeutics typically have greater amounts of AGn than AA glycans (McLEAN 2017). Without being bound by theory, it is believed that with transient expression of hGaIT vectors entirely lacking promoter and UTR elements, some T-DNAs insert into plant genome regions that both have promoter activity and provide a suitable (surrogate) UTR sequence, allowing for transcriptional starts upstream of the initial ATG codon.

Therefore, as shown herein, a healthy stable transgenic GaIT expressing plant can be produced using an expression vector that completely lacks the promoter and UTR for the GaIT coding sequence. The benefit of having such a plant production host is at least two-fold: (i) it allows for a more simplified production system, as co-infiltration of a GaIT vector would not be required for transient expression of a valuable target glycoprotein, and (ii) it allows for improved efficiency in galactosylation due to overcoming problems associated with simultaneously expressing target protein genes and post-translational modification genes in a transient process.

### Example 4

Promoters required for other PTM genes may require more activity than those entirely lacking recognizable promoter sequences and entirely lacking 5′UTR sequences such as in vector PFC1491. In Example 4, a chimeric human alpha-1,6-fucosyltransferase gene was assembled in vectors PFC1434: EE35S promoter version; PFC1455: Act2 promoter version; PFC1485: basal 35S promoter version; and PFC1486: 5′UTR version (see FIG. 7 for schematic diagrams of T-DNA regions of these vectors, and Table 4 for a description of differences of promoter and 5′UTR sequences between these vectors and the corresponding promoter-containing vectors of the hGaIT vectors of Example 3).

TABLE 4
Sequence differences in the LB to ATG start of translation codon
regions between the four FucT plasmids of FIG. 7 and the four related
hGaIT plasmids of FIG. 4.

| | hGaIT | hFucT | Comparison between hGaIT & |
|---|---|---|---|
| Promoter | plasmid | plasmid | hFucT T-DNAs |
| Double- | PFC1433 | PFC1434 | Identical functional LBs and |
| enhancer | | | associated sequences; identical |
| 35S | | | double-enhancer 35S promoters; |
| | | | PFC1433 has a 10-nt MCS deletion |
| | | | between LB and first 35S enhancer; |
| | | | 5′UTRs differ by only 3-nt (due to |
| | | | different restriction endonuclease |
| | | | cloning sites) |
| Act2 | PFC1452 | PFC1455 | Identical LB and associated |
| | | | sequences; identical Act2 promoters; |
| | | | PFC1455 has a 4-nt MCS deletion |
| | | | between LB and Act2 promoter; |
| | | | 5′UTRs differ by only 3-nt (due to |
| | | | different restriction endonuclease |
| | | | cloning sites) |
| Basal 35S-P | PFC1483 | PFC1485 | Identical LB and associated |
| | | | sequences; identical basal |
| | | | promoters; 5′UTRs differ by only |
| | | | 2-nt (due to different restriction |
| | | | endonuclease site cloning sites) |
| 5′UTR only | PFC1484 | PFC1486 | Identical LB and associated |
| | | | sequences; 5′UTRs differ by only |
| | | | 3-nt (due to different restriction |
| | | | endonuclease cloning sites) |

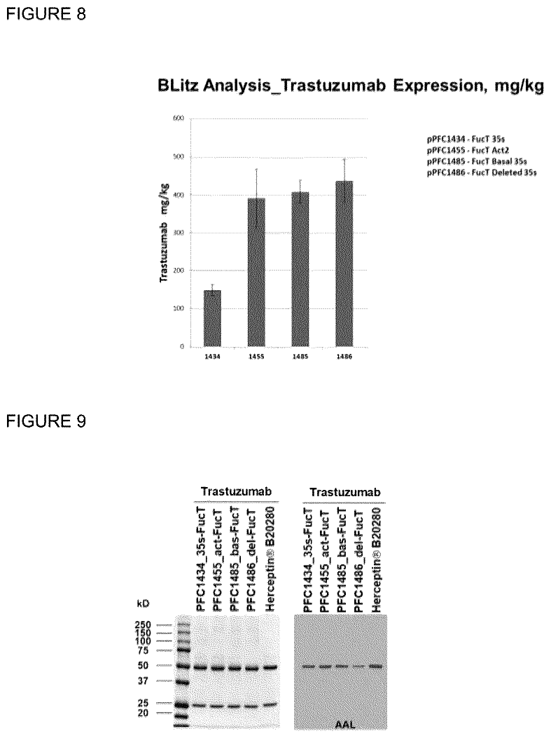

FIG. 8 shows trastuzumab antibody measurements for PFC0058 co-expression treatments with each of these four FucT vectors. Antibody measurements were performed as was described for the experiments of Example 3. As in FIG. 3, vector PFC1434 with the EE35S promoter driving FucT transcription causes reduction of antibody expression, as compared with the other three vectors. The other three vectors (PFC1455, PFC1485 and PFC1486) all show equivalent trastuzumab antibody expression.

FIG. 9, like FIG. 6, shows Coomassie blue-stained SDS-PAGE analysis of purified antibody from each of these treatments, along with a western immunoblot probed with a lectin-based reagent. Methods for this figure similar as those described for the data of FIG. 6. The key difference for this figure is that Biotinylated AAL (cat B-1395, from Vector Labs) was used as it is specific for fucose. It is also important to recall that these antibody treatments involved use of PlantForm's KDFX host plant line, which lacks detectable alpha-1,3-fucosyltransferase activity; therefore, any detectable fucosylation of antibody on the immunoblot of FIG. 9 is alpha-1,6-fucose as added glycan sugar due to the activity of the chimeric hFucT gene on the expression plasmids used in this experiment.

As can be seen in FIG. 9, biotinylated AAL detected similar amounts of fucose on antibody for three treatments; however, the fourth treatment involving PFC1486 containing the promoterless, 5′UTR-FucT vector version resulted in a fucose-specific signal of lesser intensity. This result is quantified in Table 5, showing that the PFC1486 vector resulted in (for e.g.) 31.7% GnGn glycans whereas other treatments of this experiment resulted in predominantly GnGnF glycans and less than 5% GnGn glycans. Since therapeutic antibodies typically have high amounts of alpha-1,6-fucosylation promoter variants driving FucT PTM activity that are stronger than promoterless and 5′UTR-less vectors (such as PFC1491 for hGaIT) are necessary; vectors that are promoterless, but that contain a 5′UTR may suffice (especially in the case where stable transgenic plants are produced, should the T-DNA land in a region of the plant genome that has high expressional activity); however, slightly stronger promoter variants for FucT activity may be required, such as the basal 35S promoter variant of PFC1485. The basal promoter of this vector, which contains only 96 nucleotides of the CaMV 35S promoter results in greater GnGnF glycans that does the Act2 promoter FucT vector (i.e., PFC1455). Without being bound by theory, this could be a consequence of the Act2 promoter being too strong, as this treatment resulted in 15.2% other fucosylated species, whereas the PFC1485 treatment resulted in only 8.4% other fucosylated species.

TABLE 5
Percentages of fucosylated and non-fucosylated species from above
experimental samples.

| FucT vector | PFC1455 | PFC1485 | PFC1486 |
|---|---|---|---|
| Short form | Act2-FucT | Basal 35S- | 5′UTR-FucT |
| | | FucT | |
| Antibody | 0607 | 0058 | 0058 |
| | B12 | trastuzumab | trastuzumab |
| GnGn | 4.7 | 3.0 | 31.7 |
| GnGnF | 76.7 | 84.1 | 61.4 |
| Other F spp. | 15.2 | 8.4 | 1.4 |
| Other non-F spp. | 3.5 | 4.5 | 5.5 |
| TOTAL | 100.1 | 100 | 100 |

### Example 5

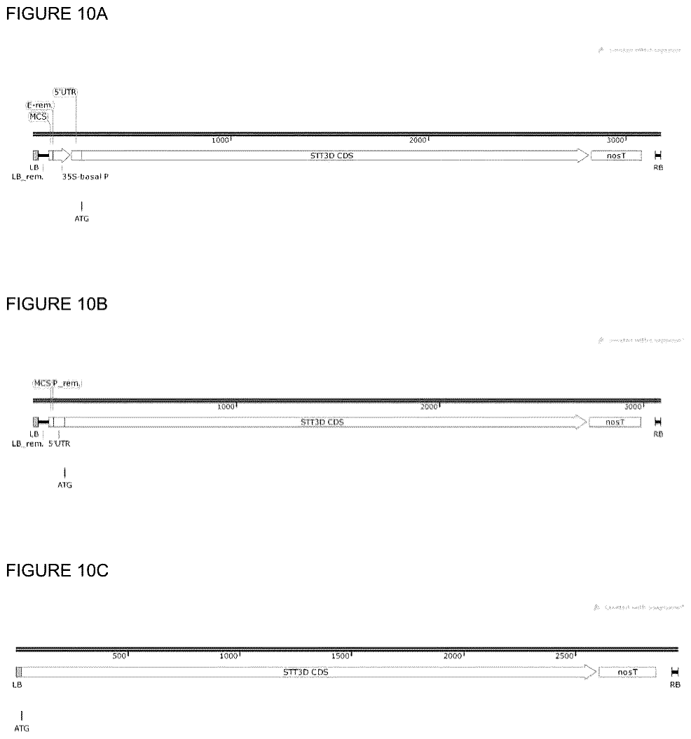

Promoters required for yet other genes encoding PTM activity, that reduce aglycosylation, may also require more activity than those entirely lacking recognizable promoter sequences and entirely lacking 5′UTR sequences such as in vector PFC1491. In Example 5, Leishmania major oligosaccharyltransferase (OTase; STT3D gene) was assembled in vectors PFC1487: basal 35S promoter version; PFC1488: 5′UTR version; and PFC1494: promoterless and 5′UTR-less version (see FIG. 10 for schematic diagrams of T-DNA regions of these vectors, and Table 6 for a description of differences of promoter and 5′UTR sequences between these vectors and the corresponding promoter-containing vectors of the hGaIT vectors of Example 3).

TABLE 6
Sequence differences between the STT3D vectors and the
corresponding GaIT vectors.

| | hGaIT | STT3D | Comparison between hGaIT & hFucT |
|---|---|---|---|
| Promoter | plasmid | plasmid | T-DNAs |
| Basal | PFC1483 | PFC1487 | Identical LB and associated |
| 35S-P | | | sequences; MCS between LB |
| | | | sequences and basal-P differ by 2 |
| | | | nucleotides (1 restriction site |
| | | | difference); identical basal promoters |
| | | | (including 4-nt enhancer remnant); |
| | | | 5′UTRs differ by only 5-nt: 4-nt due |
| | | | to different restriction endonuclease |
| | | | site cloning sites and Kozak box has |
| | | | 1 A:C transversion |
| 5′UTR | PFC1484 | PFC1488 | Identical LB and associated |
| only | | | sequences; MCS between LB |
| | | | sequences and 5′UTR differ by 2 |
| | | | nucleotides (1 restriction site |
| | | | difference); identical 5-nt basal-P |
| | | | remnant; 5′UTRs differ by only 5-nt: |
| | | | 4-nt due to different restriction |
| | | | endonuclease site cloning sites and |
| | | | Kozak box has 1 A:C transversion |
| LB-ATG | PFC1491 | PFC1494 | Identical: functional 25-nt LB is |
| | | | immediately adjacent ATG start of |
| | | | translation codon for both coding |
| | | | sequences |

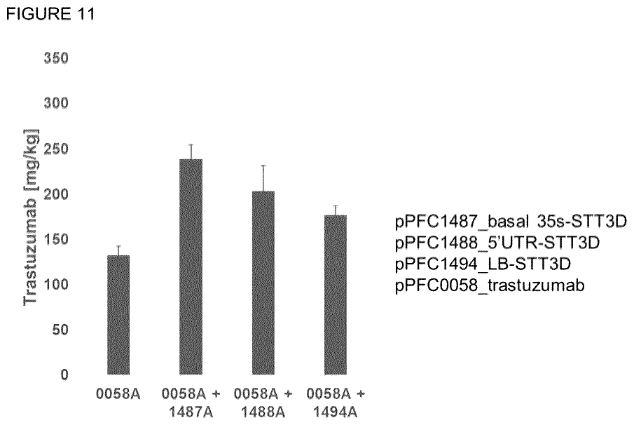

FIG. 11 shows trastuzumab antibody measurements for PFC0058 co-expression treatments with each of these three STT3D vectors. Although not shown in this figure, recall that vector PFC1480 (EE35S promoter version, diagrammed in FIG. 1D) causes reduction of antibody expression (FIG. 3). Antibody measurements were performed as was described for the experiments of Example 3. What is surprising is that vector PFC1487, containing the basal 35S promoter driving transcription of the STT3D coding sequence, increases the expression of trastuzumab antibody compared with trastuzumab expression vector PFC0058 alone, and that the other STT3D vectors of decreasing promoter strength have a diminishing although still positive effect on trastuzumab expression, as the 5′UTR version (PFC1488) has an intermediate enhancement over the promoterless and 5′UTR-less version (PFC1494).

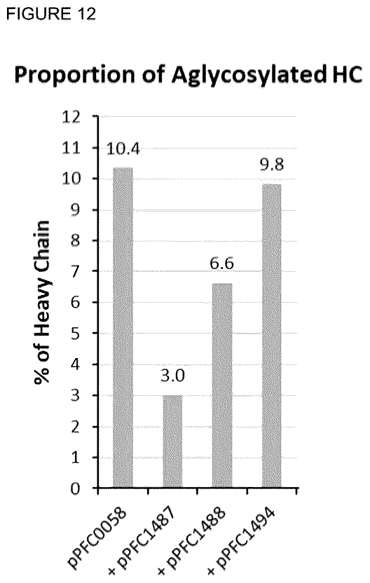

FIG. 12 shows the proportion of aglycosylated HC for these treatments. For this experiment, plant expressed antibody was purified using Ab SpinTrap (GE Healthcare). Purified antibody was dialyzed against PBS overnight at 4° C. Weak cation exchange high performance liquid chromatography (WCX-HPLC) was used to determine the proportion of aglycosylated heavy chain (HC). Each sample was injected at a flow rate of 1 mL/min into an Agilent Bio Mab, NPS, SS column (4.6×250 mm, 5 μm, P/N 5190-2405; Agilent). Agilent ChemStation software was used to calculate the peak areas of these peaks, the percent aglycosylated HC was then summarized as shown in the figure. Interestingly, trastuzumab antibody purified from vector PFC0058 alone showed slightly more than 10% HC aglycosylation, while STT3D expression vectors with increasing promoter strength showed decreasing aglycosylation; i.e., PFC1494 (promoterless and 5′UTR-less version) showed slightly less than 10% HC aglycosylation; PFC1488 (5′UTR version), 6.6% aglycosylation; PFC1487 (basal 35S promoter version), 3.0%. Thus, it appears that the basal 35S promoter driving transcription of STT3D causes the best reduction of aglycosylation while simultaneously being involved with increasing the amount of antibody expressed by plants. Table 7 shows that none of these STT3D vectors adversely affects the types of glycans post-translationally added to antibody HCs; for e.g., all four treatments of this experiment had the expected predominant glycan (i.e., GnGn) between 90% to 93%.

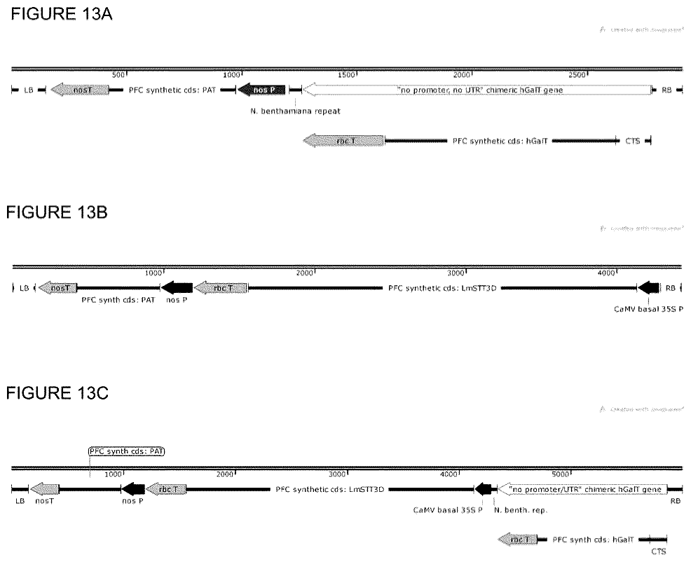

TABLE 7
Percentages of glycan species from the experiment of FIGS. 12 and 13.

| STT3D vector | none | PFC1487 | PFC1488 | PFC1494 |
|---|---|---|---|---|
| short form | none | Basal35S-STT3D | 5′UTR-STT3D | LB-STT3D |
| GnGn | 90.7 | 92.6 | 91.2 | 91.3 |
| GnM | 2.8 | 2.2 | 2.4 | 2.4 |
| Other | 6.4 | 5.2 | 6.4 | 6.3 |
| Mannosylated | | | | |
| species | | | | |
| TOTAL | 99.9 | 100 | 100 | 100 |

### Example 6

Heavy and light chain coding sequences for three different anti-HIV IgG1 antibodies (b12 (Barbas, C. F., T. A. Collet, W. Amberg, P. Roben, J. M. Binley et al., 1993 Molecular profile of an antibody response to HIV-1 as probed by combinatorial libraries. Journal of Molecular Biology 230: 812-823); PGV04 (Falkowska, E., A. Ramos, Y. Feng, T. Zhou, S. Moquin et al., 2012 PGV04, an HIV-1 gp120 CD4 binding site antibody, is broad and potent in neutralization but does not induce conformational changes characteristic of CD4. J Virol 86: 4394-4403); PGT121 (Walker, L. M., M. Huber, K. J. Doores, E. Falkowska, R. Pejchal et al., 2011 Broad neutralization coverage of HIV by multiple highly potent antibodies. Nature 477: 466-470)) were optimized for expression in plants, cloned into vivoXPRESS® vectors, and used (as described above for similar experiments) in treatments involving post-translational modification vectors Act-GaIT (PFC1452) or Act-GaIT plus Act-FucT (PFC1455). Biomass harvests occurred 7 days post-infiltration (DPI), antibodies were purified as described above (SpinTrap) and subjected to GlykoPrep analysis. Table 8 below gives mean percentage and standard deviation (S.D.) values for four classes of galactosylated glycans only: AGn or GnA; AA; AGnF or GnAF; AAF. Note that b12 expression and analysis was performed two times; therefore, data in the table below are means and S.D.'s for four independent biological repeats involving three different IgG1 antibodies. From these data, it can be seen that addition of a FucT vector to an infiltration treatment causes reductions of both AGn or GnA and AA glycans, as well as increases of AGnF or GnAF and AAF glycans. Without being bound by theory, it is believed that the use of weaker promoters as described in this application for either the GaIT and/or FucT vectors will result in similar trends for relative amounts of galactosylated and galactosylated plus fucosylated glycans on target proteins.

TABLE 8
Mean percentage and standard deviation (S.D.) values for four classes
of galactosylated glycans.

| Process | GaIT (%) | | GalT + FucT (%) | |
|---|---|---|---|---|
| Statistic | Mean | S.D. | Mean | S.D. |
| AGn or GnA | 15.4 | 5.2 | 7.2 | 0.6 |
| AGnF or GnAF | 0 | 0 | 10.7 | 2.1 |
| AA | 55.5 | 9.2 | 7.0 | 0.9 |
| AAF | 0 | 0 | 52.4 | 8.8 |

### Example 7. Production of Stable Transgenic Plants Expressing hGaIT from a Vector Entirely Lacking Promoter and UTR Elements

Methods: FIG. 13A shows a schematic diagram of the T-DNA region of vector PFC1403, containing the chimeric hGaIT coding sequence adjacent to the functional 25-nt RB sequence and a selectable marker gene (i.e., phosphinothricin acetyl transferase, PAT) for resistance to glufosinate. This vector was constructed using a combination of DNA synthesis and standard restriction endonuclease plus ligation cloning. This vector has the PAT gene (encoding phosphinothricin acetyltransferase) on the LB side and the promoter-less, UTR-less hGaIT coding sequence adjacent the RB 25 repeat.

FIG. 13B shows a schematic diagram of the T-DNA region of vector PFC1404, containing the basal 35S promoter and the STT3D coding sequence, adjacent to the functional 25-nt RB sequence and a selectable marker gene (i.e., phosphinothricin acetyl transferase, PAT) for resistance to glufosinate. This vector was constructed using a combination of DNA synthesis and standard restriction endonuclease plus ligation cloning.

FIG. 13C shows a schematic diagram of the T-DNA region of vector PFC1405, containing the chimeric hGaIT coding sequence adjacent to the functional 25-nt RB sequence; containing the basal 35S promoter and the STT3D coding sequence in the middle; and a selectable marker gene (i.e., phosphinothricin acetyl transferase, PAT) for resistance to glufosinate. This vector was constructed using a combination of DNA synthesis and standard restriction endonuclease plus ligation cloning.

Sequences of the PFC1403 and PFC1405 vectors are also set out in Table 11.

Primary stable transgenic plants have been made with PFC1403 using the procedure described below. Also, screening for hGaIT activity in offspring of primary transgenic plants has been performed using the procedure that is described further below.

To make primary stable transgenic plants with vector pPFC1403, N. benthamiana KDFX plants were raised from seed under sterile conditions. Leaves were sliced into approximately 1 cm×1 cm square pieces and exposed to Agrobacterium tumefaciens strain EHA105 harboring pPFC1403 under selective pressure involving kanamycin at 50 mg/L in the bacterial growth medium. Treated leaf pieces were placed on solid growth medium containing agarose, MS salts, vitamin B5, sucrose, naphthyl acetic acid (NAA), benzylaminopurine (BAP), timentin, plus a drug used for selection of growth by only those cells that had been transformed with T-DNA sequences of interest by the Agrobacterium. Since KDFX plants are themselves transgenic, containing T-DNA encoding RNAi cassette genes for knockdown of plant beta-1,2-xylosyltransferase and alpha-1,3-fucosyltransferase gene activities, and are thus resistant to kanamycin, therefore glufosinate (Basta®) was used for selection of growth by transformed cells with T-DNA from vector pPFC1403, as it contains a PAT gene encoding phosphinothricin acetyltransferase which would confer resistance to this herbicidal drug.

After callus formation, small shoots emerged, which were excised and transferred to solid growth medium containing agarose, MS salts, vitamin B5, sucrose, timentin, and Basta®, but lacking auxins to stimulate root growth. After formation of roots, plantlets were transferred to soil, and allowed to grow in a controlled growth room and eventually produce seed.

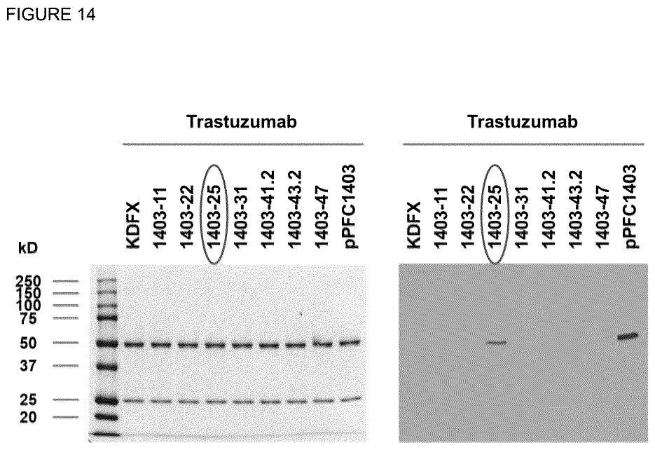

Thirty-two (32) primary transgenic (To) plants were produced using T-DNA vector pPFC1403. Twenty of those survived to maturity, were self-pollinated, and from these 20 next-generation (T₁) seed sets were collected. These T1 sibling sets were treated as families, and 2 to 6 plants from each family were infiltrated with vivoXPRESS® vector PFC0058 at about 5-6 weeks of age. Infiltrated leaf biomass was harvested 7 days post-infiltration (7 DPI) and pooled among family sets, and trastuzumab antibody was purified as described above (SpinTrap). Denaturing SDS-PAGE gels were electrophoresed with 3 μg trastuzumab samples and either stained with Coomassie blue (to confirm equivalent loading) or blotted to PVDF membrane and probed with biotinylated Ricinus communis Agglutinin I (RCA; Vector Labs, B-1085) followed by HR-conjugated streptavidin (BioLegend, cat 405210) and treatment with ECL Western Blotting Substrate for enhanced chemiluminescence detection of galactosylated heavy chains, according to manufacturer (ThermoFisher; cat. no. 32106). One (1) of 20 T1 families showed positive reactivity with the RCA lectin probe, indicating galactosylation of the trastuzumab antibody heavy chain (FIG. 14).

To quantify glycan species on glycoprotein expressed in T1 sibling plants of primary transgenic plant 1403-25, trastuzumab antibody was transiently expressed in 5 T1 plants from pPFC0058, leaf biomass was harvested 7 DPI, and trastuzumab antibody was purified by Protein G Spin Trap (GE Healthcare), as above. Glycans were prepared by using GlykoPrep Rapid N-Glycan Preparation kit (Prozyme) and relative retention times from HILIC UFLC analysis were used for identification of glycan species, also as above. Autointegration was used to calculate the quantity of each glycan species peak. Table 9 below shows glycan species quantifications on trastuzumab antibody purified from the T1 sibling plant pool from primary transgenic plant 1403-25. Note that more than 3% diantennary galactose (AA) and that more than 13% monoantennary galactose (AGn) were quantified. As these glycans are from pooled plants that have not yet been genetically characterized, it should be possible to selectively breed lines of plants from this T1 generation that homogeneously add both greater and lesser amounts of galactose to glycoproteins.

TABLE 9
Glycan species quantifications on trastuzumab antibody purified from
the T1 sibling plant pool from primary transgenic plant 1403-25.

| Glycan | 1403-25 T1 sibling plants |
|---|---|
| Species | (pool) |
| GnM | 1.570 |
| GnGn | 61.574 |
| Man4Gn/AM | 1.226 |
| AGn | 13.670 |
| Man5Gn | 0.867 |
| AA | 3.451 |
| Man7-9 | 12.642 |
| Unidentified | 5.000 |
| Total | 100.000 |

### DISCUSSION

A sufficient number of primary transgenic plants was produced and screened to allow for identification of a single plant line that could perform galactosylation of a target protein of interest. Because the PFC1403 vector was entirely lacking promoter and 5′UTR sequences, it was anticipated that the frequency of selecting transgenic plant lines with GaIT activity would be low. Without being bound by theory, GaIT activity has possibly resulted due to insertion of the PFC1403 T-DNA into a region of the N. benthamiana genome that could support weak but sufficient expression of GaIT enzyme.

Next steps for development of this plant line will involve determination of number of T-DNA insertions; determination of amounts of complex glycans (GnGn, AGn, AA type) that are post-translationally added to glycoproteins of interest, such as therapeutic antibodies; breeding to homozygosity; and confirmation of stable inheritance of GaIT activity.

TABLE 10
Sequences of vectors PFC1484, PFC1486,
PFC1488, PFC1490, PFC1492, PFC1491 and PFC1494.

| | PFC1484 | PFC1486 | PFC1488 | PFC1490 | PFC1492 | PFC1491 | PFC1494 |
|---|---|---|---|---|---|---|---|
| LB Region | SEQ ID | SEQ ID | SEQ ID | SEQ ID | SEQ ID | SEQ ID | SEQ ID |
| | NO: 1 | NO: 1 | NO: 57 | NO: 9 | NO: 12 | NO: 14 | NO: 14 |
| | Notes: | | Notes: | | | | |
| | First | | This | | | | |
| | 25-nt are | | sequence | | | | |
| | the LB | | differs | | | | |
| | sequence | | from | | | | |
| | [SEQ ID | | SEQ ID | | | | |
| | NO: 14]. | | NO: 1 | | | | |
| | The | | due to a | | | | |
| | remaining | | different | | | | |
| | 73-nt seq | | restriction | | | | |
| | consists of | | seq | | | | |
| | LB | | is at the | | | | |
| | associated | | 3′ end. | | | | |
| | sequence | | | | | | |
| | plus | | | | | | |
| | multi-cloning | | | | | | |
| | site | | | | | | |
| | sequence | | | | | | |
| | [SEQ ID | | | | | | |
| | NO: 56]. | | | | | | |
| MCS | SEQ ID NO: | SEQ ID | SEQ ID | n/a | n/a | n/a | n/a |
| | 56 | NO: 56 | NO: 58 | | | | |
| | Notes: | | Notes: | | | | |
| | These are | | These | | | | |
| | Asel, Ascl | | are Asel, | | | | |
| | and Xhol | | Ascl | | | | |
| | restriction | | and Sall | | | | |
| | sites. This | | restriction | | | | |
| | seq is the 3′ | | sites. | | | | |
| | end of | | | | | | |
| | SEQ ID | | | | | | |
| | NO: 1. | | | | | | |
| Promoter | SEQ ID | SEQ ID | SEQ ID | SEQ ID | SEQ ID | n/a | n/a |
| sequence | NO: 2 | NO: 2 | NO: 2 | NO: 10 | NO: 10 | | |
| remainder | Notes: This | | Notes: | | | | |
| | is the | | This | | | | |
| | remainder | | sequence | | | | |
| | of the 35S | | is | | | | |
| | promoter. | | duplicated | | | | |
| | There are | | at | | | | |
| | 73 nt | | the 5′ | | | | |
| | between | | end of | | | | |
| | this 5-nt | | SEQ ID | | | | |
| | promoter | | NO: 7 | | | | |
| | remnant | | | | | | |
| | and the | | | | | | |
| | functional | | | | | | |
| | LB 25-nt | | | | | | |
| | seq (73+25 | | | | | | |
| | nt seq is | | | | | | |
| | SEQ ID | | | | | | |
| | NO: 1) | | | | | | |
| 5′UTR | SEQ ID | SEQ ID | SEQ ID | SEQ ID | SEQ ID | n/a | n/a |
| | NO: 3 | NO: 5 | NO: 7 | NO: 3 | NO: 3 | | |
| | | Notes: | | | | | |
| | | Difference | | | | | |
| | | from | | | | | |
| | | SEQ ID | | | | | |
| | | NO: 3 | | | | | |
| | | is due to | | | | | |
| | | use of a | | | | | |
| | | different | | | | | |
| | | restriction | | | | | |
| | | site at the | | | | | |
| | | 3′ end of | | | | | |
| | | this | | | | | |
| | | sequence | | | | | |
| START | ATG | ATG | ATG | ATG | ATG | ATG | ATG |
| ″LB | SEQ ID | SEQ ID | SEQ ID | SEQ ID | SEQ ID | SEQ ID | SEQ ID |
| sequences | NO: 4 | NO: 6 | NO: 8 | NO: 11 | NO: 13 | NO: 15 | NO: 15 |
| to up to | Notes: This | | | | | | |
| and | 157 nt | | | | | | |
| including | sequence | | | | | | |
| ATG start″ | consists of | | | | | | |
| | (L to R): LB | | | | | | |
| | sequence | | | | | | |
| | (25 nt) + LB | | | | | | |
| | assoc seq | | | | | | |
| | incl MCS | | | | | | |
| | (73 nt) + | | | | | | |
| | promoter | | | | | | |
| | remnant (5 | | | | | | |
| | nt) +5′utr | | | | | | |
| | (51 nt) + | | | | | | |
| | ATG (3 nt) | | | | | | |
| PTM | GaIT | FucT | STT3D | GaIT | GaIT | GaIT | STT3D |
| ENZYME | [SEQ ID | [SEQ ID | [SEQ ID | [SEQ ID | [SEQ ID | [SEQ ID | [SEQ ID |
| | NO: 17] | NO: 21] | NO: 19] | NO: 17] | NO: 17] | NO: 17] | NO: 19] |

TABLE 11
Sequences of vectors PFC1403 and PFC1405.

| | PFC1403 | PFC1405 |
|---|---|---|
| LB Region | SEQ ID NO: 76 | SEQ ID NO: 76 |
| MCS | SEQ ID NO: 77 | SEQ ID NO: 77 |
| reverse complement of nos terminator = | SEQ ID NO: 78 | SEQ ID NO: 78 |
| terminator sequence of nopaline synthase | | |
| gene | | |
| PFC synthetic seq: PAT (phosphinothricin | SEQ ID NO: 79 | SEQ ID NO: 79 |
| acetyltransferase) coding sequence; reverse | | |
| complement | | |
| cloning site | SEQ ID NO: 80 | SEQ ID NO: 80 |
| reverse complement of nos promoter = | SEQ ID NO: 81 | SEQ ID NO: 81 |
| promoter of nopaline synthase gene | | |
| multi cloning site | SEQ ID NO: 82 | A synthetic DNA |
| | | insertion of 3079 nt |
| | | [SEQ ID NO: 92] was |
| | | inserted between the |
| | | 12th and 13th nts of |
| | | multicloning site SEQ |
| | | ID NO: 82 |
| N. benthamiana repeat “B” consensus | SEQ ID NO: 83 | SEQ ID NO: 83 |
| sequence | | |
| cloning site | SEQ ID NO: 84 | SEQ ID NO: 84 |
| reverse complement of rbcT = terminator of | SEQ ID NO: 85 | SEQ ID NO: 85 |
| rubisco gene | | |
| cloning site | SEQ ID NO: 86 | SEQ ID NO: 86 |
| PFC synthetic seq: hGalT; n.b.reverse | SEQ ID NO: 87 | SEQ ID NO: 87 |
| complement | | |
| PFC synthetic seq: CTS; n.b. reverse | SEQ ID NO: 88 | SEQ ID NO: 88 |
| complement | | |
| cloning site | SEQ ID NO: 89 | SEQ ID NO: 89 |
| RB sequence | SEQ ID NO: 90 | SEQ ID NO: 90 |
| RB region; n.b. that this includes the 25 nt RB | SEQ ID NO: 91 | SEQ ID NO: 91 |
| sequence (SEQ ID NO: 90); Agrobacterium | | |
| tumefaciens Ti plasmid pTiC58 T-DNA region | | |

TABLE 12
Description of sequences.

| SEQ | | |
|---|---|---|
| ID No | DESCRIPTION | Sequence |
| 1 | LB sequence +20 nt multi | TGGCAGGATATATTGTGGTGTAAACAAATTGACGCTT |
| | cloning site of PF01484 | AGACAACTTAATAACACATTGCGGACGTTTTTAATGTA |
| | and PF01486 | CTGATTAATGGCGCGCCCTCGAG |
| 2 | Promoter sequence | AGAGG |
| | remainder of PF01484, | |
| | PF01486 and PF01488 | |
| 3 | 5′ UTR of PF01484, | ACACGCTGAAATCACCAGTCTCTCTCTACAAATCTATC |
| | PF01490 and PFC 1492 | TCTGGCGCCAAAA |
| 4 | PF01484 sequence-LB | TGGCAGGATATATTGTGGTGTAAACAAATTGACGCTT |
| | sequence to and including | AGACAACTTAATAACACATTGCGGACGTTTTTAATGTA |
| | ATG start | CTGATTAATGGCGCGCCCTCGAGAGAGGACACGCTG |
| | | AAATCACCAGTCTCTCTCTACAAATCTATCTCTGGCGC |
| | | CAAAAATG |
| 5 | 5′ UTR of PF01486 | ACACGCTGAAATCACCAGTCTCTCTCTACAAATCTATC |
| | | TCTGAGCTCAAAA |
| 6 | PF01486 sequence-LB | TGGCAGGATATATTGTGGTGTAAACAAATTGACGCTT |
| | sequence to and including | AGACAACTTAATAACACATTGCGGACGTTTTTAATGTA |
| | ATG start | CTGATTAATGGCGCGCCCTCGAGAGAGGACACGCTG |
| | | AAATCACCAGTCTCTCTCTACAAATCTATCTCTGAGCT |
| | | CAAAAATG |
| 7 | 5′ UTR of PF01488, | AGAGGACACGCTGAAATCACCAGTCTCTCTCTACAAA |
| | includes AGAGG at its 5′ | TCTATCTCTGAGCTCAACA |
| | end. Has much of the 35S | |
| | UTR with slight | |
| | differences at the 3′ end | |
| | where a SalI site was | |
| | engineered for cloning | |
| | purposes | |
| 8 | PF01488 sequence-LB | TGGCAGGATATATTGTGGTGTAAACAAATTGACGCTT |
| | sequence to and including | AGACAACTTAATAACACATTGCGGACGTTTTTAATGTA |
| | ATG start | CTGATTAATGGCGCGCCGTCGACAGAGGACACGCTG |
| | | AAATCACCAGTCTCTCTCTACAAATCTATCTCTGAGCT |
| | | CAACAATG |
| 9 | LB region of PF01490 | TGGCAGGATATATTGTGGTGTAAACAAATTGA |
| 10 | Promoter sequence | GAGAGG |
| | remainder of PF01490 | |
| 11 | PF01490 sequence-LB | TGGCAGGATATATTGTGGTGTAAACAAATTGAGAGAG |
| | sequence to and including | GACACGCTGAAATCACCAGTCTCTCTCTACAAATCTAT |
| | ATG start | CTCTGGCGCCAAAAATG |
| 12 | LB region of PF01492 | TGGCAGGATATATTGTGGTGTAAACGA |
| 13 | PF01492 sequence-LB | TGGCAGGATATATTGTGGTGTAAACGAGAGAGGACAC |
| | sequence to and including | GCTGAAATCACCAGTCTCTCTCTACAAATCTATCTCTG |
| | ATG start | GCGCCAAAAATG |
| 14 | LB sequence of PF01491 | TGGCAGGATATATTGTGGTGTAAAC |
| | and PF01494 | |
| 15 | PF01491 and PF01494 | TGGCAGGATATATTGTGGTGTAAACATG |
| | sequence-LB sequence | |
| | up to and including ATG | |
| | start | |
| 16 | human GalT amino acid | AAAIGQSSGELRTGGARPPPPLGASSQPRPGGDSSPV |
| | sequence | VDSGPGPASNLTSVPVPHTTALSLPACPEESPLLVGPM |
| | | LIEFNMPVDLELVAKQNPNVKMGGRYAPRDCVSPHKVA |
| | | IIIPFRNRQEHLKYWLYYLHPVLQRQQLDYGIYVINQAGD |
| | | TIFNRAKLLNVGFQEALKDYDYTCFVFSDVDLIPMNDHN |
| | | AYRCFSQPRHISVAMDKFGFSLPYVQYFGGVSALSKQQ |
| | | FLTINGFPNNYWGWGGEDDDIFNRLVFRGMSISRPNAV |
| | | VGRCRMIRHSRDKKNEPNPQRFDRIAHTKETMLSDGLN |
| | | SLTYQVLDVQRYPLYTQITVDIGTPS |
| 17 | 1155 bp chimeric GalT | ATGATTCACACGAACCTGAAGAAGAAGTTCAGCCTCT |
| | coding sequence. | TCATCCTGGTTTTCCTGCTCTTCGCGGTAATCTGCGT |
| | Contains at its 5′ end the | TTGGAAGAAGGGTTCTGACTACGAAGCCCTCACCCTC |
| | coding sequence for the | CAGGCGAAGGAATTCCAGATGCCGAAGTCTCAGGAG |
| | CTS domain of the rat | AAGGTTGCCGCAGCCATCGGTCAGTCCTCTGGTGAA |
| | alpha-2,6- | CTCCGTACCGGTGGTGCTCGTCCTCCACCGCCGCTG |
| | sialyltransferase | GGTGCATCTAGCCAGCCGCGTCCGGGTGGCGACAG |
| | | CTCTCCGGTTGTGGATTCTGGCCCAGGTCCAGCTTCT |
| | | AACCTGACGTCTGTTCCGGTTCCACATACCACCGCGC |
| | | TCAGCCTGCCGGCGTGCCCGGAAGAATCTCCGCTGC |
| | | TGGTAGGCCCTATGCTCATCGAATTCAACATGCCGGT |
| | | AGACCTGGAACTCGTTGCGAAGCAGAACCCGAACGT |
| | | AAAGATGGGTGGTCGCTACGCCCCTCGTGATTGCGT |
| | | TTCCCCGCACAAGGTGGCCATCATCATTCCTTTCCGT |
| | | AACCGTCAAGAGCACCTGAAATACTGGCTGTACTACC |
| | | TGCACCCGGTTCTGCAGCGTCAGCAGCTCGACTACG |
| | | GTATCTACGTTATCAACCAGGCGGGTGACACCATCTT |
| | | TAACCGCGCTAAACTGCTGAACGTGGGTTTCCAGGA |
| | | GGCGCTCAAGGATTACGACTACACCTGCTTCGTTTTC |
| | | TCTGACGTTGACCTGATCCCGATGAATGATCACAACG |
| | | CCTACCGTTGCTTTTCTCAACCACGTCACATCTCTGTT |
| | | GCGATGGACAAATTCGGTTTCTCTCTCCCGTATGTAC |
| | | AGTACTTCGGTGGCGTGTCTGCCCTCTCTAAGCAGCA |
| | | ATTCCTGACGATCAACGGTTTCCCGAACAATTACTGG |
| | | GGTTGGGGTGGTGAAGACGATGATATCTTCAACCGC |
| | | CTCGTATTCCGCGGTATGTCTATCAGCCGTCCGAATG |
| | | CGGTCGTGGGCCGCTGCCGTATGATCCGTCACAGCC |
| | | GTGACAAGAAGAACGAGCCGAACCCGCAGCGCTTTG |
| | | ACCGTATCGCGCACACCAAAGAAACTATGCTGTCTGA |
| | | CGGCCTGAACTCTCTCACGTACCAAGTTCTCGACGTA |
| | | CAGCGTTACCCGCTGTATACCCAGATCACCGTCGACA |
| | | TCGGTACCCCGTCTTGATGA |
| 18 | Leishmania STT3D amino | MGKRKGNSLGDSGSAATASREASAQAEDAASQTKTAS |
| | acid sequence | PPAKVILLPKTLTDEKDFIGIFPFPFWPVHFVLTVVALFVL |
| | | AASCFQAFTVRMISVQIYGYLIHEFDPWFNYRAAEYMST |
| | | HGWSAFFSWFDYMSWYPLGRPVGSTTYPGLQLTAVAI |
| | | HRALAAAGMPMSLNNVCVLMPAWFGAIATATLAFCTYE |
| | | ASGSTVAAAAAALSFSIIPAHLMRSMAGEFDNECIAVAA |
| | | MLLTFYCWVRSLRTRSSWPIGVLTGVAYGYMAAAWGG |
| | | YIFVLNMVAMHAGISSMVDWARNTYNPSLLRAYTLFYVV |
| | | GTAIAVCVPPVGMSPFKSLEQLGALLVLVFLCGLQVCEV |
| | | LRARAGVEVRSRANFKIRVRVFSVMAGVAALAISVLAPT |
| | | GYFGPLSVRVRALFVEHTRTGNPLVDSVAEHQPASPEA |
| | | MWAFLHVCGVTWGLGSIVLAVSTFVHYSPSKVFWLLNS |
| | | GAVYYFSTRMARLLLLSGPAACLSTGIFVGTILEAAVQLS |
| | | FWDSDATKAKKQQKQAQRHQRGAGKGSGRDDAKNAT |
| | | TARAFCDVFAGSSLAWGHRMVLSIAMWALVTTTAVSFF |
| | | SSEFASHSTKFAEQSSNPMIVFAAVVQNRATGKPMNLL |
| | | VDDYLKAYEWLRDSTPEDARVLAWWDYGYQITGIGNRT |
| | | SLADGNTWNHEHIATIGKMLTSPVVEAHSLVRHMADYV |
| | | LIWAGQSGDLMKSPHMARIGNSVYHDICPDDPLCQQFG |
| | | FHRNDYSRPTPMMRASLLYNLHEAGKRKGVKVNPSLF |
| | | QEVYSSKYGLVRIFKVMNVSAESKKWVADPANRVCHPP |
| | | GSWICPGQYPPAKEIQEMLAHRVPFDQVTNADRKNNV |
| | | GSYQEEYMRRMRESENRR |
| 19 | 2574 bp STT3D coding | ATGGGTAAGCGTAAGGGCAACAGCCTTGGTGATTCT |
| | sequence (synthetic, plant | GGTTCTGCTGCTACCGCTTCTAGAGAGGCTTCTGCTC |
| | optimized version of the | AAGCTGAAGATGCTGCTTCTCAGACCAAGACTGCTAG |
| | LmSTT3D polypeptide of | CCCTCCTGCTAAGGTTATCCTGCTTCCTAAGACCTTG |
| | SEQ ID NO: 19) | ACCGACGAGAAGGACTTTATCGGGATCTTCCCTTTTC |
| | | CGTTCTGGCCTGTGCATTTCGTGCTTACTGTTGTGGC |
| | | TCTTTTCGTGCTGGCTGCTTCTTGCTTTCAGGCTTTCA |
| | | CCGTGAGGATGATCAGCGTGCAGATCTACGGTTACCT |
| | | GATCCACGAGTTCGACCCGTGGTTTAATTACAGGGCT |
| | | GCCGAGTACATGTCTACCCATGGTTGGTCTGCTTTCT |
| | | TCAGCTGGTTCGACTACATGAGCTGGTATCCTCTTGG |
| | | TAGGCCTGTGGGTTCTACTACTTATCCTGGACTTCAG |
| | | CTTACCGCTGTGGCTATTCATAGAGCTTTGGCTGCTG |
| | | CTGGCATGCCGATGTCTCTTAACAATGTGTGCGTGCT |
| | | GATGCCTGCATGGTTCGGTGCTATTGCTACTGCTACT |
| | | TTGGCCTTCTGTACCTACGAGGCTTCAGGTTCTACTG |
| | | TTGCTGCTGCAGCTGCTGCTCTGAGCTTCTCTATTATT |
| | | CCTGCTCACCTGATGCGGAGCATGGCTGGTGAATTT |
| | | GACAACGAGTGCATTGCTGTGGCTGCTATGCTTCTGA |
| | | CTTTCTACTGCTGGGTGAGATCCCTTAGGACCAGATC |
| | | TTCTTGGCCTATTGGTGTGCTTACCGGTGTTGCTTAC |
| | | GGTTACATGGCTGCAGCTTGGGGCGGTTACATTTTCG |
| | | TGTTGAACATGGTGGCTATGCACGCCGGCATTAGCTC |
| | | TATGGTTGATTGGGCTCGTAATACTTACAACCCGAGC |
| | | CTTCTTAGGGCTTACACCCTTTTCTACGTGGTGGGAA |
| | | CCGCTATTGCTGTTTGTGTTCCTCCTGTGGGCATGAG |
| | | CCCTTTCAAGTCTCTTGAACAGCTTGGTGCTCTGCTG |
| | | GTGCTTGTTTTCTTGTGCGGACTTCAGGTTTGCGAGG |
| | | TGTTGAGAGCTAGAGCTGGTGTTGAGGTTAGGTCCA |
| | | GGGCTAACTTCAAGATCAGAGTGAGGGTGTTCTCCGT |
| | | TATGGCTGGCGTTGCAGCTCTTGCTATTTCTGTGCTT |
| | | GCTCCTACCGGTTACTTCGGTCCTTTGTCTGTTAGGG |
| | | TGAGAGCCTTGTTCGTTGAGCATACCAGGACTGGTAA |
| | | CCCTCTGGTTGATTCTGTTGCTGAGCATCAGCCTGCT |
| | | TCTCCAGAGGCTATGTGGGCTTTTCTTCATGTGTGCG |
| | | GTGTGACTTGGGGTCTGGGTTCTATTGTGTTGGCTGT |
| | | GTCTACCTTCGTGCACTACAGCCCTTCTAAGGTGTTC |
| | | TGGCTTCTGAACTCTGGCGCCGTGTACTACTTCTCTA |
| | | CTAGGATGGCTAGGCTCCTGCTTCTTTCTGGACCTGC |
| | | TGCTTGTCTTAGCACCGGTATTTTCGTGGGCACCATT |
| | | CTTGAAGCTGCCGTGCAGTTGTCTTTCTGGGATTCTG |
| | | ATGCTACCAAGGCCAAAAAGCAGCAAAAGCAGGCTC |
| | | AGAGGCATCAGAGAGGTGCTGGTAAAGGTTCTGGTA |
| | | GGGATGACGCTAAGAATGCTACTACCGCTCGGGCTTT |
| | | CTGTGATGTGTTTGCTGGTTCTTCTCTGGCTTGGGGT |
| | | CACCGTATGGTGCTTTCTATTGCAATGTGGGCTCTTG |
| | | TGACTACCACCGCCGTTTCTTTCTTCTCCTCCGAATTC |
| | | GCTTCCCACAGCACTAAGTTCGCTGAGCAGTCAAGCA |
| | | ACCCGATGATTGTGTTCGCTGCTGTTGTGCAGAATCG |
| | | TGCTACTGGCAAGCCTATGAACCTGCTGGTGGATGAT |
| | | TACCTGAAGGCTTACGAGTGGCTGAGGGATTCTACTC |
| | | CTGAGGATGCTAGAGTTCTCGCTTGGTGGGATTACG |
| | | GCTACCAGATTACCGGTATTGGCAACAGGACCTCTCT |
| | | GGCTGATGGTAATACTTGGAACCACGAGCACATTGCC |
| | | ACCATCGGTAAGATGCTTACTAGCCCTGTTGTCGAGG |
| | | CTCACTCTCTTGTTAGGCACATGGCTGATTACGTGCT |
| | | GATTTGGGCTGGTCAGTCTGGCGATCTTATGAAGTCT |
| | | CCTCACATGGCTAGGATCGGCAACTCTGTGTACCACG |
| | | ATATCTGCCCTGATGATCCTCTTTGCCAGCAGTTCGG |
| | | TTTCCACCGGAATGATTACTCTCGGCCTACTCCTATG |
| | | ATGCGGGCTTCTCTTCTTTACAACCTTCACGAGGCTG |
| | | GTAAGCGGAAAGGTGTTAAGGTGAACCCGAGCTTGTT |
| | | CCAAGAGGTGTACAGCTCTAAGTACGGCCTGGTGAG |
| | | GATCTTCAAGGTGATGAATGTGAGCGCCGAGAGCAA |
| | | GAAGTGGGTTGCAGATCCTGCTAATAGGGTGTGCCAT |
| | | CCTCCTGGTTCTTGGATTTGTCCTGGTCAGTACCCTC |
| | | CGGCCAAAGAAATTCAAGAGATGCTGGCTCATAGGGT |
| | | GCCGTTCGATCAGGTTACCAACGCTGATCGGAAGAA |
| | | CAACGTGGGGTCTTATCAAGAGGAGTACATGCGGAG |
| | | GATGCGTGAGTCTGAGAATAGAAGGTAA |
| 20 | Chimeric FucT aa | MRSASNSNAPNKQWRNWLPLFVALVIIAEFSFLVRLDVA |
| | sequence. The predicted | EVRONDHPDHSSRELSKILAKLERLKQQNEDLRRMAES |
| | 39 N-terminal aa′s are | LRIPEGPIDQGPAIGRVRVLEEQLVKAKEQIENYKKQTR |
| | identical to the N | NGLGKDHEILRRRIENGAKELWFFLQSELKKLKNLEGNE |
| | benthamiana FucT1. | LQRHADEFLLDLGHHERSIMTDLYYLSQTDGAGDWREK |
| | signal peptide; the 546 C- | EAKDLTELVQRRITYLQNPKDCSKAKKLVCNINKGCGYG |
| | terminal aa′s are identical | CQLHHVVYCFMIAYGTQRTLILESQNWRYATGGWETVF |
| | to human alpha-1,6- | RPVSETCTDRSGISTGHWSGEVKDKNVQVVELPIVDSL |
| | fucosyltransferase. | HPRPPYLPLAVPEDLADRLVRVHGDPAVWWVSQFVKY |
| | | LIRPQPWLEKEIEEATKKLGFKHPVIGVHVRRTDKVGTE |
| | | AAFHPIEEYMVHVEEHFQLLARRMQVDKKRVYLATDDP |
| | | SLLKEAKTKYPNYEFISDNSISWSAGLHNRYTENSLRGVI |
| | | LDIHFLSQADFLVCTFSSQVCRVAYEIMQTLHPDASANF |
| | | HSLDDIYYFGGQNAHNQIAIYAHQPRTADEIPMEPGDIIG |
| | | VAGNHWDGYSKGVNRKLGRTGLYPSYKVREKIETVKYP |
| | | TYPEAEK* |
| 21 | Nucleotide sequence for | ATGAGGTCTGCTTCTAATTCTAACGCTCCAAACAAGC |
| | Chimeric UCT aa | AATGGAGGAACTGGCTTCCACTTTTCGTGGCTCTTGT |
| | sequence | GATCATCGCTGAATTCTCTTTCTTGGTTAGATTGGACG |
| | | TTGCAGAGGTGAGGGACAACGACCACCCAGATCACT |
| | | CATCTAGGGAGTTGTCTAAAATCCTTGCTAAATTGGAA |
| | | AGGTTGAAACAACAAAATGAGGACTTGAGGAGGATG |
| | | GCTGAGTCTTTGAGAATCCCAGAGGGACCTATCGACC |
| | | AAGGACCAGCAATCGGTAGGGTGAGAGTGTTGGAGG |
| | | AGCAGCTTGTTAAGGCAAAGGAGCAAATCGAAAACTA |
| | | CAAGAAGCAGACTAGGAACGGATTGGGAAAGGACCA |
| | | CGAAATCCTTAGGAGGAGAATCGAGAACGGAGCTAA |
| | | GGAACTTTGGTTTTTCCTTCAATCAGAGTTGAAGAAGT |
| | | TGAAGAATTTGGAAGGTAACGAGTTGCAGAGACACGC |
| | | TGACGAGTTCCTTCTTGATTTGGGTCACCACGAGAGG |
| | | TCAATCATGACTGACTTGTACTATTTGTCTCAGACTGA |
| | | CGGTGCTGGAGACTGGAGAGAGAAGGAGGCTAAGGA |
| | | CTTGACTGAGCTTGTGCAGAGGAGAATTACATATCTT |
| | | CAAAACCCAAAAGATTGTTCAAAAGCAAAGAAGTTGG |
| | | TGTGCAATATCAACAAGGGATGCGGATACGGATGTCA |
| | | GTTGCACCACGTTGTGTACTGCTTCATGATTGCTTAC |
| | | GGAACTCAGAGGACTTTGATTCTTGAATCTCAAAACT |
| | | GGAGGTACGCTACAGGTGGATGGGAAACAGTGTTCA |
| | | GGCCAGTGTCTGAGACATGCACAGACAGGTCTGGTA |
| | | TCTCAACAGGTCACTGGTCTGGAGAGGTGAAGGACA |
| | | AGAACGTGCAGGTGGTTGAGTTGCCTATCGTTGACTC |
| | | ATTGCACCCAAGGCCACCTTACTTGCCACTTGCAGTT |
| | | CCTGAGGACTTGGCTGACAGGCTTGTTAGGGTGCAT |
| | | GGAGATCCTGCAGTGTGGTGGGTGTCACAGTTTGTG |
| | | AAGTACCTTATCAGACCACAGCCATGGTTGGAGAAAG |
| | | AGATCGAGGAGGCAACTAAGAAGCTTGGTTTCAAACA |
| | | TCCAGTGATCGGAGTGCACGTGAGGAGGACTGACAA |
| | | GGTGGGAACTGAAGCAGCATTCCACCCTATTGAGGA |
| | | GTACATGGTGCACGTGGAGGAGCACTTTCAGTTGCTT |
| | | GCAAGGAGGATGCAGGTGGACAAAAAGAGGGTGTAC |
| | | CTTGCTACAGATGACCCATCTCTTCTTAAAGAGGCTA |
| | | AGACTAAATACCCTAATTATGAGTTCATCTCAGACAAC |
| | | TCTATTTCATGGTCAGCTGGATTGCATAATAGATATAC |
| | | TGAAAACTCACTTAGGGGAGTTATTTTGGATATTCATT |
| | | TCCTTTCTCAGGCTGATTTCTTGGTTTGTACTTTCTCT |
| | | TCACAAGTTTGTAGAGTGGCTTACGAGATCATGCAGA |
| | | CACTTCACCCAGATGCTTCTGCTAATTTCCACTCTTTG |
| | | GACGATATTTATTATTTCGGTGGTCAAAATGCACATAA |
| | | CCAAATTGCAATTTACGCTCATCAGCCAAGGACTGCT |
| | | GACGAGATTCCAATGGAGCCTGGAGACATCATCGGT |
| | | GTGGCAGGAAACCACTGGGATGGTTACTCAAAGGGA |
| | | GTGAACAGGAAATTGGGTAGAACTGGTCTTTATCCTT |
| | | CTTACAAGGTGAGGGAAAAGATCGAGACAGTGAAATA |
| | | CCCTACATACCCAGAGGCAGAGAAGTGA |
| 22 | 148 bp LB region. T-DNA | CTGATGGGCTGCCTGTATCGAGTGGTGATTTTGTGCC |
| | left border: GenBank | GAGCTGCCGGTCGGGGAGCTGTTGGCTGGCTGGTG |
| | Accession Number | GCAGGATATATTGTGGTGTAAACAAATTGACGCTTAG |
| | J01825; 25-nt LB seq is | ACAACTTAATAACACATTGCGGACGTTTTTAATGTACT |
| | embedded within this | G |
| | sequence. | |
| 23 | 25 bp LB sequence; | TGGCAGGATATATTGTGGTGTAAAC |
| | 100% identity with | |
| | GenBank accession | |
| | Sequence ID: | |
| | AJ237588.1; contained in | |
| | plasmids 1433, 1483, | |
| | 1484, 1490, 1492, 1491, | |
| | 1452 | |
| 24 | 162 bp RB region. T-DNA | AGATTGTCGTTTCCCGCCTTCAGTTTAAACTATCAGTG |
| | right border: GenBank | TTTGACAGGATATATTGGCGGGTAAACCTAAGAGAAA |
| | Accession Number | AGAGCGTTTATTAGAATAATCGGATATTTAAAAGGGC |
| | J01826; 25-nt RB | GTGAAAAGGTTTATCCGTTCGTCCATTTGTATGTGCAT |
| | sequence is embedded | GCCAACCACAGG |
| | within this sequence. | |
| 25 | 25 bp RB sequence. | TGACAGGATATATTGGCGGGTAAAC |
| | Right border repeat from | |
| | nopaline C58 T-DNA. | |
| 26 | 5′UTR sequence. 5′UTR | ACACGCTGAAATCACCAGTCTCTCTCTACAAATCTATC |
| | of CaMV 35S RNA gene; | TCTGGCGCCAAAA |
| | 3 end of which is modified | |
| | to contain a KasI cloning | |
| | site and the 5′ end of a | |
| | Kozak box. | |
| 27 | 325 bp 35S enhancer | AACATGGTGGAGCACGACACTCTCGTCTACTCCAAGA |
| | sequence. 100 % | ATATCAAAGATACAGTCTCAGAAGACCAAAGGGCTAT |
| | sequence identity with | TGAGACTTTTCAACAAAGGGTAATATCGGGAAACCTC |
| | Cauliflower mosaic virus | CTCGGATTCCATTGCCCAGCTATCTGTCACTTCATCA |
| | genome Sequence ID: | AAAGGACAGTAGAAAAGGAAGGTGGCACCTACAAAT |
| | gi\|58815\|V00140.1Length: | GCCATCATTGCGATAAAGGAAAGGCTATCGTTCAAGA |
| | 8031 | TGCCTCTGCCGACAGTGGTCCCAAAGATGGACCCCC |
| | | ACCCACGAGGAGCATCGTGGAAAAAGAAGACGTTCC |
| | | AACCACGTCTTCAAAGCAAGTGGATTGATGTG |
| 28 | 92 bp 35S basal promoter | ATATCTCCACTGACGTAAGGGATGACGCACAATCCCA |
| | sequence. 100 % | CTATCCTTCGCAAGACCCTTCCTCTATATAAGGAAGTT |
| | sequence identity with | CATTTCATTTGGAGAGG |
| | Cauliflower mosaic virus | |
| | genome Sequence ID: | |
| | gi\|58815\|V00140.1Length: | |
| | 8031 | |
| 29 | P19 CDS. This is the PFC | ATGGAAAGGGCTATTCAGGGAAATGATGCTAGAGAG |
| | synthetic cds for P19. No | CAGGCTAATTCTGAAAGATGGGATGGTGGATCTGGTG |
| | detectable similarity with | GAACTACTTCTCCATTCAAGCTTCCAGATGAGTCTCC |
| | the GenBank entry that | ATCTTGGACTGAGTGGAGGCTTCATAACGATGAGACT |
| | provides the cds for P19 | AACTCCAATCAGGATAACCCACTCGGATTCAAAGAAT |
| | (Tomato bushy stunt virus | CTTGGGGATTCGGAAAGGTTGTGTTCAAGCGTTACCT |
| | isolate TBSVEgh p22 | TAGGTATGATAGGACTGAGGCTTCACTTCATAGGGTT |
| | protein gene, complete | CTCGGATCTTGGACTGGTGATTCTGTTAACTACGCTG |
| | cds GenBank: | CTTCTCGTTTTTTTGGATTCGATCAGATCGGATGCACT |
| | JX418297.1) | TACTCTATTAGGTTCAGGGGAGTGTCTATTACTGTTTC |
| | | TGGTGGATCTAGGACTCTTCAACACCTTTGCGAGATG |
| | | GCTATTAGGTCTAAGCAAGAGCTTCTTCAGCTTGCTC |
| | | CAATTGAGGTTGAGTCTAACGTTTCAAGAGGATGTCC |
| | | AGAAGGTACTGAGACTTTCGAGAAAGAATCCGAGTGA |
| | | AAATTGACGCTTAGACAACTTAATAACACATTGCGGA |
| 30 | 53 nt 3′ of LB sequence: | CGTTTTTAATGTACTG |
| | 100% identity with | |
| | GenBank accession | |
| | Sequence ID: | |
| | gi\|5042179\|AJ237588.1; | |
| | contained in plasmids | |
| | 1433, 1483, 1484 | |
| 31 | 7-nt from 5′ end of SEQ ID | AAATTGA |
| | NO: 30 | |
| 32 | AseI to BsiWI multi- | ATTAATGGCGCGCCCTCGAGGCCCCGTACG |
| | cloning site | |
| 33 | AseI to XhoI multi-cloning | ATTAATGGCGCGCCCTCGAG |
| | site | |
| 34 | 2-nt cloning artefact | GA |
| 35 | AseI to DraII multi-cloning | ATTAATGGCGCGCCCTCGAGGCCC |
| | site | |
| 36 | 35Sp romoter enhancer | AACATGGTGGAGCACGACACTCTCGTCTACTCCAAGA |
| | sequence | ATATCAAAGATACAGTCTCAGAAGACCAAAGGGCTAT |
| | | TGAGACTTTTCAACAAAGGGTAATATCGGGAAACCTC |
| | | CTCGGATTCCATTGCCCAGCTATCTGTCACTTCATCA |
| | | AAAGGACAGTAGAAAAGGAAGGTGGCACCTACAAAT |
| | | GCCATCATTGCGATAAAGGAAAGGCTATCGTTCAAGA |
| | | TGCCTCTGCCGACAGTGGTCCCAAAGATGGACCCCC |
| | | ACCCACGAGGAGCATCGTGGAAAAAGAAGACGTTCC |
| | | AACCACGTCTTCAAAGCAAGTGGATTGATGTG |
| 37 | 35S basal promoter | ATATCTCCACTGACGTAAGGGATGACGCACAATCCCA |
| | | CTATCCTTCGCAAGACCCTTCCTCTATATAAGGAAGTT |
| | | CATTTCATTTGGAGAGG |
| 38 | 6-nt from 3′ end of 35S | GAGAGG |
| | basal promoter | |
| 39 | 35S 5′ untranslated region | ACACGCTGAAATCACCAGTCTCTCTCTACAAATCTATC |
| | (UTR), modified to contain | TCTGGCGCCAAAA |
| | KasI restriction site | |
| 40 | Modified Arabidopsis | MAKTNLFLFLIFSLLLSLSSA |
| | thaliana basic chitinase | |
| | signal peptide | |
| 41 | native human | MHSKVTIICIRFLFWFLLLCMLIGKSHT |
| | butyrylcholinesterase | |
| | signal peptide. 100% | |
| | identical to (28/28 aas) | |
| | butyrylcholinesterase, | |
| | isoform CRA_b [Homo | |
| | sapiens] | |
| | Sequence ID: | |
| | EAW78592.1 | |
| 42 | 1433 full T-DNA | CTGATGGGCTGCCTGTATCGAGTGGTGATTTTGTGCC |
| | sequence, including LB | GAGCTGCCGGTCGGGGAGCTGTTGGCTGGCTGGTG |
| | region and RB region as | GCAGGATATATTGTGGTGTAAACAAATTGACGCTTAG |
| | given in original pBIN19 | ACAACTTAATAACACATTGCGGACGTTTTTAATGTACT |
| | publication (BEVAN 1984) | GATTAATGGCGCGCCCTCGAGGCCCCGTACGAACAT |
| | | GGTGGAGCACGACACTCTCGTCTACTCCAAGAATATC |
| | | AAAGATACAGTCTCAGAAGACCAAAGGGCTATTGAGA |
| | | CTTTTCAACAAAGGGTAATATCGGGAAACCTCCTCGG |
| | | ATTCCATTGCCCAGCTATCTGTCACTTCATCAAAAGGA |
| | | CAGTAGAAAAGGAAGGTGGCACCTACAAATGCCATCA |
| | | TTGCGATAAAGGAAAGGCTATCGTTCAAGATGCCTCT |
| | | GCCGACAGTGGTCCCAAAGATGGACCCCCACCCACG |
| | | AGGAGCATCGTGGAAAAAGAAGACGTTCCAACCACG |
| | | TCTTCAAAGCAAGTGGATTGATGTGAACATGGTGGAG |
| | | CACGACACTCTCGTCTACTCCAAGAATATCAAAGATA |
| | | CAGTCTCAGAAGACCAAAGGGCTATTGAGACTTTTCA |
| | | ACAAAGGGTAATATCGGGAAACCTCCTCGGATTCCAT |
| | | TGCCCAGCTATCTGTCACTTCATCAAAAGGACAGTAG |
| | | AAAAGGAAGGTGGCACCTACAAATGCCATCATTGCGA |
| | | TAAAGGAAAGGCTATCGTTCAAGATGCCTCTGCCGAC |
| | | AGTGGTCCCAAAGATGGACCCCCACCCACGAGGAGC |
| | | ATCGTGGAAAAAGAAGACGTTCCAACCACGTCTTCAA |
| | | AGCAAGTGGATTGATGTGATATCTCCACTGACGTAAG |
| | | GGATGACGCACAATCCCACTATCCTTCGCAAGACCCT |
| | | TCCTCTATATAAGGAAGTTCATTTCATTTGGAGAGGAC |
| | | ACGCTGAAATCACCAGTCTCTCTCTACAAATCTATCTC |
| | | TGGCGCCAAAAATGATTCACACGAACCTGAAGAAGAA |
| | | GTTCAGCCTCTTCATCCTGGTTTTCCTGCTCTTCGCG |
| | | GTAATCTGCGTTTGGAAGAAGGGTTCTGACTACGAAG |
| | | CCCTCACCCTCCAGGCGAAGGAATTCCAGATGCCGA |
| | | AGTCTCAGGAGAAGGTTGCCGCAGCCATCGGTCAGT |
| | | CCTCTGGTGAACTCCGTACCGGTGGTGCTCGTCCTC |
| | | CACCGCCGCTGGGTGCATCTAGCCAGCCGCGTCCGG |
| | | GTGGCGACAGCTCTCCGGTTGTGGATTCTGGCCCAG |
| | | GTCCAGCTTCTAACCTGACGTCTGTTCCGGTTCCACA |
| | | TACCACCGCGCTCAGCCTGCCGGCGTGCCCGGAAGA |
| | | ATCTCCGCTGCTGGTAGGCCCTATGCTCATCGAATTC |
| | | AACATGCCGGTAGACCTGGAACTCGTTGCGAAGCAG |
| | | AACCCGAACGTAAAGATGGGTGGTCGCTACGCCCCT |
| | | CGTGATTGCGTTTCCCCGCACAAGGTGGCCATCATCA |
| | | TTCCTTTCCGTAACCGTCAAGAGCACCTGAAATACTG |
| | | GCTGTACTACCTGCACCCGGTTCTGCAGCGTCAGCA |
| | | GCTCGACTACGGTATCTACGTTATCAACCAGGCGGGT |
| | | GACACCATCTTTAACCGCGCTAAACTGCTGAACGTGG |
| | | GTTTCCAGGAGGCGCTCAAGGATTACGACTACACCTG |
| | | CTTCGTTTTCTCTGACGTTGACCTGATCCCGATGAAT |
| | | GATCACAACGCCTACCGTTGCTTTTCTCAACCACGTC |
| | | ACATCTCTGTTGCGATGGACAAATTCGGTTTCTCTCTC |
| | | CCGTATGTACAGTACTTCGGTGGCGTGTCTGCCCTCT |
| | | CTAAGCAGCAATTCCTGACGATCAACGGTTTCCCGAA |
| | | CAATTACTGGGGTTGGGGTGGTGAAGACGATGATATC |
| | | TTCAACCGCCTCGTATTCCGCGGTATGTCTATCAGCC |
| | | GTCCGAATGCGGTCGTGGGCCGCTGCCGTATGATCC |
| | | GTCACAGCCGTGACAAGAAGAACGAGCCGAACCCGC |
| | | AGCGCTTTGACCGTATCGCGCACACCAAAGAAACTAT |
| | | GCTGTCTGACGGCCTGAACTCTCTCACGTACCAAGTT |
| | | CTCGACGTACAGCGTTACCCGCTGTATACCCAGATCA |
| | | CCGTCGACATCGGTACCCCGTCTTGATGAAGATCTTC |
| | | CGGATCGATAATGAAATGTAAGAGATATCATATATAAA |
| | | TAATAAATTGTCGTTTCATATTTGCAATCTTTTTTTTAC |
| | | AAACCTTTAATTAATTGTATGTATGACATTTTCTTCTTG |
| | | TTATATTAGGGGGAAATAATGTTAAATAAAAGTACAAA |
| | | ATAAACTACAGTACATCGTACTGAATAAATTACCTAGC |
| | | CAAAAAGTACACCTTTCCATATACTTCCTACATGAAGG |
| | | CATTTTCAACATTTTCAAATAAGGAATGCTACAACCGC |
| | | ATAATAACATCCACAAATTTTTTTATAAAATAACATGTC |
| | | AGACAGTGATTGAAAGATTTTATTATAGTTTCGTTATC |
| | | TTGCTAGCGGCCGGCCTTAATTAAAGATTGTCGTTTC |
| | | CCGCCTTCAGTTTAAACTATCAGTGTTTGACAGGATAT |
| | | ATTGGCGGGTAAACCTAAGAGAAAAGAGCGTTTATTA |
| | | GAATAATCGGATATTTAAAAGGGCGTGAAAAGGTTTA |
| | | TCCGTTCGTCCATTTGTATGTGCATGCCAACCACAGG |
| 43 | 1433 LB sequence to ATG | TGGCAGGATATATTGTGGTGTAAACAAATTGACGCTT |
| | start of translation | AGACAACTTAATAACACATTGCGGACGTTTTTAATGTA |
| | (inclusive) | CTGATTAATGGCGCGCCCTCGAGGCCCCGTACGAAC |
| | | ATGGTGGAGCACGACACTCTCGTCTACTCCAAGAATA |
| | | TCAAAGATACAGTCTCAGAAGACCAAAGGGCTATTGA |
| | | GACTTTTCAACAAAGGGTAATATCGGGAAACCTCCTC |
| | | GGATTCCATTGCCCAGCTATCTGTCACTTCATCAAAA |
| | | GGACAGTAGAAAAGGAAGGTGGCACCTACAAATGCC |
| | | ATCATTGCGATAAAGGAAAGGCTATCGTTCAAGATGC |
| | | CTCTGCCGACAGTGGTCCCAAAGATGGACCCCCACC |
| | | CACGAGGAGCATCGTGGAAAAAGAAGACGTTCCAAC |
| | | CACGTCTTCAAAGCAAGTGGATTGATGTGAACATGGT |
| | | GGAGCACGACACTCTCGTCTACTCCAAGAATATCAAA |
| | | GATACAGTCTCAGAAGACCAAAGGGCTATTGAGACTT |
| | | TTCAACAAAGGGTAATATCGGGAAACCTCCTCGGATT |
| | | CCATTGCCCAGCTATCTGTCACTTCATCAAAAGGACA |
| | | GTAGAAAAGGAAGGTGGCACCTACAAATGCCATCATT |
| | | GCGATAAAGGAAAGGCTATCGTTCAAGATGCCTCTGC |
| | | CGACAGTGGTCCCAAAGATGGACCCCCACCCACGAG |
| | | GAGCATCGTGGAAAAAGAAGACGTTCCAACCACGTCT |
| | | TCAAAGCAAGTGGATTGATGTGATATCTCCACTGACG |
| | | TAAGGGATGACGCACAATCCCACTATCCTTCGCAAGA |
| | | CCCTTCCTCTATATAAGGAAGTTCATTTCATTTGGAGA |
| | | GGACACGCTGAAATCACCAGTCTCTCTCTACAAATCT |
| | | ATCTCTGGCGCCAAAAATG |
| 44 | 1483 full T-DNA | CTGATGGGCTGCCTGTATCGAGTGGTGATTTTGTGCC |
| | sequence, including LB | GAGCTGCCGGTCGGGGAGCTGTTGGCTGGCTGGTG |
| | region and RB region as | GCAGGATATATTGTGGTGTAAACAAATTGACGCTTAG |
| | given in original pBIN19 | ACAACTTAATAACACATTGCGGACGTTTTTAATGTACT |
| | publication (BEVAN 1984) | GATTAATGGCGCGCCCTCGAGTGTGATATCTCCACTG |
| | | ACGTAAGGGATGACGCACAATCCCACTATCCTTCGCA |
| | | AGACCCTTCCTCTATATAAGGAAGTTCATTTCATTTGG |
| | | AGAGGACACGCTGAAATCACCAGTCTCTCTCTACAAA |
| | | TCTATCTCTGGCGCCAAAAATGATTCACACGAACCTG |
| | | AAGAAGAAGTTCAGCCTCTTCATCCTGGTTTTCCTGC |
| | | TCTTCGCGGTAATCTGCGTTTGGAAGAAGGGTTCTGA |
| | | CTACGAAGCCCTCACCCTCCAGGCGAAGGAATTCCA |
| | | GATGCCGAAGTCTCAGGAGAAGGTTGCCGCAGCCAT |
| | | CGGTCAGTCCTCTGGTGAACTCCGTACCGGTGGTGC |
| | | TCGTCCTCCACCGCCGCTGGGTGCATCTAGCCAGCC |
| | | GCGTCCGGGTGGCGACAGCTCTCCGGTTGTGGATTC |
| | | TGGCCCAGGTCCAGCTTCTAACCTGACGTCTGTTCCG |
| | | GTTCCACATACCACCGCGCTCAGCCTGCCGGCGTGC |
| | | CCGGAAGAATCTCCGCTGCTGGTAGGCCCTATGCTC |
| | | ATCGAATTCAACATGCCGGTAGACCTGGAACTCGTTG |
| | | CGAAGCAGAACCCGAACGTAAAGATGGGTGGTCGCT |
| | | ACGCCCCTCGTGATTGCGTTTCCCCGCACAAGGTGG |
| | | CCATCATCATTCCTTTCCGTAACCGTCAAGAGCACCT |
| | | GAAATACTGGCTGTACTACCTGCACCCGGTTCTGCAG |
| | | CGTCAGCAGCTCGACTACGGTATCTACGTTATCAACC |
| | | AGGCGGGTGACACCATCTTTAACCGCGCTAAACTGCT |
| | | GAACGTGGGTTTCCAGGAGGCGCTCAAGGATTACGA |
| | | CTACACCTGCTTCGTTTTCTCTGACGTTGACCTGATC |
| | | CCGATGAATGATCACAACGCCTACCGTTGCTTTTCTC |
| | | AACCACGTCACATCTCTGTTGCGATGGACAAATTCGG |
| | | TTTCTCTCTCCCGTATGTACAGTACTTCGGTGGCGTG |
| | | TCTGCCCTCTCTAAGCAGCAATTCCTGACGATCAACG |
| | | GTTTCCCGAACAATTACTGGGGTTGGGGTGGTGAAG |
| | | ACGATGATATCTTCAACCGCCTCGTATTCCGCGGTAT |
| | | GTCTATCAGCCGTCCGAATGCGGTCGTGGGCCGCTG |
| | | CCGTATGATCCGTCACAGCCGTGACAAGAAGAACGA |
| | | GCCGAACCCGCAGCGCTTTGACCGTATCGCGCACAC |
| | | CAAAGAAACTATGCTGTCTGACGGCCTGAACTCTCTC |
| | | ACGTACCAAGTTCTCGACGTACAGCGTTACCCGCTGT |
| | | ATACCCAGATCACCGTCGACATCGGTACCCCGTCTTG |
| | | ATGAAGATCTTCCGGATCGATAATGAAATGTAAGAGA |
| | | TATCATATATAAATAATAAATTGTCGTTTCATATTTGCA |
| | | ATCTTTTTTTTACAAACCTTTAATTAATTGTATGTATGA |
| | | CATTTTCTTCTTGTTATATTAGGGGGAAATAATGTTAA |
| | | ATAAAAGTACAAAATAAACTACAGTACATCGTACTGAA |
| | | TAAATTACCTAGCCAAAAAGTACACCTTTCCATATACT |
| | | TCCTACATGAAGGCATTTTCAACATTTTCAAATAAGGA |
| | | ATGCTACAACCGCATAATAACATCCACAAATTTTTTTA |
| | | TAAAATAACATGTCAGACAGTGATTGAAAGATTTTATT |
| | | ATAGTTTCGTTATCTTGCTAGCGGCCGGCCTTAATTAA |
| | | AGATTGTCGTTTCCCGCCTTCAGTTTAAACTATCAGTG |
| | | TTTGACAGGATATATTGGCGGGTAAACCTAAGAGAAA |
| | | AGAGCGTTTATTAGAATAATCGGATATTTAAAAGGGC |
| | | GTGAAAAGGTTTATCCGTTCGTCCATTTGTATGTGCAT |
| | | GCCAACCACAGG |
| 45 | 1483 LB sequence to ATG | TGGCAGGATATATTGTGGTGTAAACAAATTGACGCTT |
| | start of translation | AGACAACTTAATAACACATTGCGGACGTTTTTAATGTA |
| | (inclusive) | CTGATTAATGGCGCGCCCTCGAGTGTGATATCTCCAC |
| | | TGACGTAAGGGATGACGCACAATCCCACTATCCTTCG |
| | | CAAGACCCTTCCTCTATATAAGGAAGTTCATTTCATTT |
| | | GGAGAGGACACGCTGAAATCACCAGTCTCTCTCTACA |
| | | AATCTATCTCTGGCGCCAAAAATG |
| 46 | 1484 full T-DNA | CTGATGGGCTGCCTGTATCGAGTGGTGATTTTGTGCC |
| | sequence, including LB | GAGCTGCCGGTCGGGGAGCTGTTGGCTGGCTGGTG |
| | region and RB region as | GCAGGATATATTGTGGTGTAAACAAATTGACGCTTAG |
| | given in original pBIN19 | ACAACTTAATAACACATTGCGGACGTTTTTAATGTACT |
| | publication (BEVAN 1984) | GATTAATGGCGCGCCCTCGAGAGAGGACACGCTGAA |
| | | ATCACCAGTCTCTCTCTACAAATCTATCTCTGGCGCC |
| | | AAAAATGATTCACACGAACCTGAAGAAGAAGTTCAGC |
| | | CTCTTCATCCTGGTTTTCCTGCTCTTCGCGGTAATCTG |
| | | CGTTTGGAAGAAGGGTTCTGACTACGAAGCCCTCACC |
| | | CTCCAGGCGAAGGAATTCCAGATGCCGAAGTCTCAG |
| | | GAGAAGGTTGCCGCAGCCATCGGTCAGTCCTCTGGT |
| | | GAACTCCGTACCGGTGGTGCTCGTCCTCCACCGCCG |
| | | CTGGGTGCATCTAGCCAGCCGCGTCCGGGTGGCGAC |
| | | AGCTCTCCGGTTGTGGATTCTGGCCCAGGTCCAGCTT |
| | | CTAACCTGACGTCTGTTCCGGTTCCACATACCACCGC |
| | | GCTCAGCCTGCCGGCGTGCCCGGAAGAATCTCCGCT |
| | | GCTGGTAGGCCCTATGCTCATCGAATTCAACATGCCG |
| | | GTAGACCTGGAACTCGTTGCGAAGCAGAACCCGAAC |
| | | GTAAAGATGGGTGGTCGCTACGCCCCTCGTGATTGC |
| | | GTTTCCCCGCACAAGGTGGCCATCATCATTCCTTTCC |
| | | GTAACCGTCAAGAGCACCTGAAATACTGGCTGTACTA |
| | | CCTGCACCCGGTTCTGCAGCGTCAGCAGCTCGACTA |
| | | CGGTATCTACGTTATCAACCAGGCGGGTGACACCATC |
| | | TTTAACCGCGCTAAACTGCTGAACGTGGGTTTCCAGG |
| | | AGGCGCTCAAGGATTACGACTACACCTGCTTCGTTTT |
| | | CTCTGACGTTGACCTGATCCCGATGAATGATCACAAC |
| | | GCCTACCGTTGCTTTTCTCAACCACGTCACATCTCTG |
| | | TTGCGATGGACAAATTCGGTTTCTCTCTCCCGTATGT |
| | | ACAGTACTTCGGTGGCGTGTCTGCCCTCTCTAAGCAG |
| | | CAATTCCTGACGATCAACGGTTTCCCGAACAATTACT |
| | | GGGGTTGGGGTGGTGAAGACGATGATATCTTCAACC |
| | | GCCTCGTATTCCGCGGTATGTCTATCAGCCGTCCGAA |
| | | TGCGGTCGTGGGCCGCTGCCGTATGATCCGTCACAG |
| | | CCGTGACAAGAAGAACGAGCCGAACCCGCAGCGCTT |
| | | TGACCGTATCGCGCACACCAAAGAAACTATGCTGTCT |
| | | GACGGCCTGAACTCTCTCACGTACCAAGTTCTCGACG |
| | | TACAGCGTTACCCGCTGTATACCCAGATCACCGTCGA |
| | | CATCGGTACCCCGTCTTGATGAAGATCTTCCGGATCG |
| | | ATAATGAAATGTAAGAGATATCATATATAAATAATAAAT |
| | | TGTCGTTTCATATTTGCAATCTTTTTTTTACAAACCTTT |
| | | AATTAATTGTATGTATGACATTTTCTTCTTGTTATATTA |
| | | GGGGGAAATAATGTTAAATAAAAGTACAAAATAAACTA |
| | | CAGTACATCGTACTGAATAAATTACCTAGCCAAAAAGT |
| | | ACACCTTTCCATATACTTCCTACATGAAGGCATTTTCA |
| | | ACATTTTCAAATAAGGAATGCTACAACCGCATAATAAC |
| | | ATCCACAAATTTTTTTATAAAATAACATGTCAGACAGT |
| | | GATTGAAAGATTTTATTATAGTTTCGTTATCTTGCTAG |
| | | CGGCCGGCCTTAATTAAAGATTGTCGTTTCCCGCCTT |
| | | CAGTTTAAACTATCAGTGTTTGACAGGATATATTGGCG |
| | | GGTAAACCTAAGAGAAAAGAGCGTTTATTAGAATAAT |
| | | CGGATATTTAAAAGGGCGTGAAAAGGTTTATCCGTTC |
| | | GTCCATTTGTATGTGCATGCCAACCACAGG |
| 47 | 1484 LB sequence to ATG | TGGCAGGATATATTGTGGTGTAAACAAATTGACGCTT |
| | start of translation | AGACAACTTAATAACACATTGCGGACGTTTTTAATGTA |
| | (inclusive) | CTGATTAATGGCGCGCCCTCGAGAGAGGACACGCTG |
| | | AAATCACCAGTCTCTCTCTACAAATCTATCTCTGGCGC |
| | | CAAAAATG |
| 48 | 1490 full T-DNA | CTGATGGGCTGCCTGTATCGAGTGGTGATTTTGTGCC |
| | sequence, including LB | GAGCTGCCGGTCGGGGAGCTGTTGGCTGGCTGGTG |
| | region and RB region as | GCAGGATATATTGTGGTGTAAACAAATTGAGAGAGGA |
| | given in original pBIN19 | CACGCTGAAATCACCAGTCTCTCTCTACAAATCTATCT |
| | publication (BEVAN 1984) | CTGGCGCCAAAAATGATTCACACGAACCTGAAGAAGA |
| | | AGTTCAGCCTCTTCATCCTGGTTTTCCTGCTCTTCGC |
| | | GGTAATCTGCGTTTGGAAGAAGGGTTCTGACTACGAA |
| | | GCCCTCACCCTCCAGGCGAAGGAATTCCAGATGCCG |
| | | AAGTCTCAGGAGAAGGTTGCCGCAGCCATCGGTCAG |
| | | TCCTCTGGTGAACTCCGTACCGGTGGTGCTCGTCCTC |
| | | CACCGCCGCTGGGTGCATCTAGCCAGCCGCGTCCGG |
| | | GTGGCGACAGCTCTCCGGTTGTGGATTCTGGCCCAG |
| | | GTCCAGCTTCTAACCTGACGTCTGTTCCGGTTCCACA |
| | | TACCACCGCGCTCAGCCTGCCGGCGTGCCCGGAAGA |
| | | ATCTCCGCTGCTGGTAGGCCCTATGCTCATCGAATTC |
| | | AACATGCCGGTAGACCTGGAACTCGTTGCGAAGCAG |
| | | AACCCGAACGTAAAGATGGGTGGTCGCTACGCCCCT |
| | | CGTGATTGCGTTTCCCCGCACAAGGTGGCCATCATCA |
| | | TTCCTTTCCGTAACCGTCAAGAGCACCTGAAATACTG |
| | | GCTGTACTACCTGCACCCGGTTCTGCAGCGTCAGCA |
| | | GCTCGACTACGGTATCTACGTTATCAACCAGGCGGGT |
| | | GACACCATCTTTAACCGCGCTAAACTGCTGAACGTGG |
| | | GTTTCCAGGAGGCGCTCAAGGATTACGACTACACCTG |
| | | CTTCGTTTTCTCTGACGTTGACCTGATCCCGATGAAT |
| | | GATCACAACGCCTACCGTTGCTTTTCTCAACCACGTC |
| | | ACATCTCTGTTGCGATGGACAAATTCGGTTTCTCTCTC |
| | | CCGTATGTACAGTACTTCGGTGGCGTGTCTGCCCTCT |
| | | CTAAGCAGCAATTCCTGACGATCAACGGTTTCCCGAA |
| | | CAATTACTGGGGTTGGGGTGGTGAAGACGATGATATC |
| | | TTCAACCGCCTCGTATTCCGCGGTATGTCTATCAGCC |
| | | GTCCGAATGCGGTCGTGGGCCGCTGCCGTATGATCC |
| | | GTCACAGCCGTGACAAGAAGAACGAGCCGAACCCGC |
| | | AGCGCTTTGACCGTATCGCGCACACCAAAGAAACTAT |
| | | GCTGTCTGACGGCCTGAACTCTCTCACGTACCAAGTT |
| | | CTCGACGTACAGCGTTACCCGCTGTATACCCAGATCA |
| | | CCGTCGACATCGGTACCCCGTCTTGATGAAGATCTTC |
| | | CGGATCGATAATGAAATGTAAGAGATATCATATATAAA |
| | | TAATAAATTGTCGTTTCATATTTGCAATCTTTTTTTTAC |
| | | AAACCTTTAATTAATTGTATGTATGACATTTTCTTCTTG |
| | | TTATATTAGGGGGAAATAATGTTAAATAAAAGTACAAA |
| | | ATAAACTACAGTACATCGTACTGAATAAATTACCTAGC |
| | | CAAAAAGTACACCTTTCCATATACTTCCTACATGAAGG |
| | | CATTTTCAACATTTTCAAATAAGGAATGCTACAACCGC |
| | | ATAATAACATCCACAAATTTTTTTATAAAATAACATGTC |
| | | AGACAGTGATTGAAAGATTTTATTATAGTTTCGTTATC |
| | | TTGCTAGCGGCCGGCCTTAATTAAAGATTGTCGTTTC |
| | | CCGCCTTCAGTTTAAACTATCAGTGTTTGACAGGATAT |
| | | ATTGGCGGGTAAACCTAAGAGAAAAGAGCGTTTATTA |
| | | GAATAATCGGATATTTAAAAGGGCGTGAAAAGGTTTA |
| | | TCCGTTCGTCCATTTGTATGTGCATGCCAACCACAGG |
| 49 | 1490 LB sequence to ATG | TGGCAGGATATATTGTGGTGTAAACAAATTGAGAGAG |
| | start of translation | GACACGCTGAAATCACCAGTCTCTCTCTACAAATCTAT |
| | (inclusive) | CTCTGGCGCCAAAAATG |
| 50 | 1492 full T-DNA | CTGATGGGCTGCCTGTATCGAGTGGTGATTTTGTGCC |
| | sequence, including LB | GAGCTGCCGGTCGGGGAGCTGTTGGCTGGCTGGTG |
| | region and RB region as | GCAGGATATATTGTGGTGTAAACGAGAGAGGACACG |
| | given in original pBIN19 | CTGAAATCACCAGTCTCTCTCTACAAATCTATCTCTGG |
| | publication (BEVAN 1984) | CGCCAAAAATGATTCACACGAACCTGAAGAAGAAGTT |
| | | CAGCCTCTTCATCCTGGTTTTCCTGCTCTTCGCGGTA |
| | | ATCTGCGTTTGGAAGAAGGGTTCTGACTACGAAGCCC |
| | | TCACCCTCCAGGCGAAGGAATTCCAGATGCCGAAGT |
| | | CTCAGGAGAAGGTTGCCGCAGCCATCGGTCAGTCCT |
| | | CTGGTGAACTCCGTACCGGTGGTGCTCGTCCTCCAC |
| | | CGCCGCTGGGTGCATCTAGCCAGCCGCGTCCGGGT |
| | | GGCGACAGCTCTCCGGTTGTGGATTCTGGCCCAGGT |
| | | CCAGCTTCTAACCTGACGTCTGTTCCGGTTCCACATA |
| | | CCACCGCGCTCAGCCTGCCGGCGTGCCCGGAAGAAT |
| | | CTCCGCTGCTGGTAGGCCCTATGCTCATCGAATTCAA |
| | | CATGCCGGTAGACCTGGAACTCGTTGCGAAGCAGAA |
| | | CCCGAACGTAAAGATGGGTGGTCGCTACGCCCCTCG |
| | | TGATTGCGTTTCCCCGCACAAGGTGGCCATCATCATT |
| | | CCTTTCCGTAACCGTCAAGAGCACCTGAAATACTGGC |
| | | TGTACTACCTGCACCCGGTTCTGCAGCGTCAGCAGCT |
| | | CGACTACGGTATCTACGTTATCAACCAGGCGGGTGAC |
| | | ACCATCTTTAACCGCGCTAAACTGCTGAACGTGGGTT |
| | | TCCAGGAGGCGCTCAAGGATTACGACTACACCTGCTT |
| | | CGTTTTCTCTGACGTTGACCTGATCCCGATGAATGAT |
| | | CACAACGCCTACCGTTGCTTTTCTCAACCACGTCACA |
| | | TCTCTGTTGCGATGGACAAATTCGGTTTCTCTCTCCC |
| | | GTATGTACAGTACTTCGGTGGCGTGTCTGCCCTCTCT |
| | | AAGCAGCAATTCCTGACGATCAACGGTTTCCCGAACA |
| | | ATTACTGGGGTTGGGGTGGTGAAGACGATGATATCTT |
| | | CAACCGCCTCGTATTCCGCGGTATGTCTATCAGCCGT |
| | | CCGAATGCGGTCGTGGGCCGCTGCCGTATGATCCGT |
| | | CACAGCCGTGACAAGAAGAACGAGCCGAACCCGCAG |
| | | CGCTTTGACCGTATCGCGCACACCAAAGAAACTATGC |
| | | TGTCTGACGGCCTGAACTCTCTCACGTACCAAGTTCT |
| | | CGACGTACAGCGTTACCCGCTGTATACCCAGATCACC |
| | | GTCGACATCGGTACCCCGTCTTGATGAAGATCTTCCG |
| | | GATCGATAATGAAATGTAAGAGATATCATATATAAATA |
| | | ATAAATTGTCGTTTCATATTTGCAATCTTTTTTTTACAA |
| | | ACCTTTAATTAATTGTATGTATGACATTTTCTTCTTGTT |
| | | ATATTAGGGGGAAATAATGTTAAATAAAAGTACAAAAT |
| | | AAACTACAGTACATCGTACTGAATAAATTACCTAGCCA |
| | | AAAAGTACACCTTTCCATATACTTCCTACATGAAGGCA |
| | | TTTTCAACATTTTCAAATAAGGAATGCTACAACCGCAT |
| | | AATAACATCCACAAATTTTTTTATAAAATAACATGTCAG |
| | | ACAGTGATTGAAAGATTTTATTATAGTTTCGTTATCTT |
| | | GCTAGCGGCCGGCCTTAATTAAAGATTGTCGTTTCCC |
| | | GCCTTCAGTTTAAACTATCAGTGTTTGACAGGATATAT |
| | | TGGCGGGTAAACCTAAGAGAAAAGAGCGTTTATTAGA |
| | | ATAATCGGATATTTAAAAGGGCGTGAAAAGGTTTATC |
| | | CGTTCGTCCATTTGTATGTGCATGCCAACCACAGG |
| 51 | 1492 LB sequence to ATG | TGGCAGGATATATTGTGGTGTAAACGAGAGAGGACAC |
| | start of translation | GCTGAAATCACCAGTCTCTCTCTACAAATCTATCTCTG |
| | (inclusive) | GCGCCAAAAATG |
| 52 | chimeric hGalT used in | ATGATTCACACGAACCTGAAGAAGAAGTTCAGCCTCT |
| | PF01403 and PF01405; | TCATCCTGGTTTTCCTGCTCTTCGCGGTAATCTGCGT |
| | differs by 2 nucleotides | TTGGAAGAAGGGTTCTGACTACGAAGCCCTCACCCTC |
| | with SEQ17 of this table, | CAGGCGAAGGAATTCCAGATGCCGAAGTCTCAGGAG |
| | so as to remove KpnI and | AAGGTTGCCGCAGCCATCGGTCAGTCCTCTGGTGAA |
| | SalI restriction sites from | CTCCGTACCGGTGGTGCTCGTCCTCCACCGCCGCTG |
| | original sequence | GGTGCATCTAGCCAGCCGCGTCCGGGTGGCGACAG |
| | | CTCTCCGGTTGTGGATTCTGGCCCAGGTCCAGCTTCT |
| | | AACCTGACGTCTGTTCCGGTTCCACATACCACCGCGC |
| | | TCAGCCTGCCGGCGTGCCCGGAAGAATCTCCGCTGC |
| | | TGGTAGGCCCTATGCTCATCGAATTCAACATGCCGGT |
| | | AGACCTGGAACTCGTTGCGAAGCAGAACCCGAACGT |
| | | AAAGATGGGTGGTCGCTACGCCCCTCGTGATTGCGT |
| | | TTCCCCGCACAAGGTGGCCATCATCATTCCTTTCCGT |
| | | AACCGTCAAGAGCACCTGAAATACTGGCTGTACTACC |
| | | TGCACCCGGTTCTGCAGCGTCAGCAGCTCGACTACG |
| | | GTATCTACGTTATCAACCAGGCGGGTGACACCATCTT |
| | | TAACCGCGCTAAACTGCTGAACGTGGGTTTCCAGGA |
| | | GGCGCTCAAGGATTACGACTACACCTGCTTCGTTTTC |
| | | TCTGACGTTGACCTGATCCCGATGAATGATCACAACG |
| | | CCTACCGTTGCTTTTCTCAACCACGTCACATCTCTGTT |
| | | GCGATGGACAAATTCGGTTTCTCTCTCCCGTATGTAC |
| | | AGTACTTCGGTGGCGTGTCTGCCCTCTCTAAGCAGCA |
| | | ATTCCTGACGATCAACGGTTTCCCGAACAATTACTGG |
| | | GGTTGGGGTGGTGAAGACGATGATATCTTCAACCGC |
| | | CTCGTATTCCGCGGTATGTCTATCAGCCGTCCGAATG |
| | | CGGTCGTGGGCCGCTGCCGTATGATCCGTCACAGCC |
| | | GTGACAAGAAGAACGAGCCGAACCCGCAGCGCTTTG |
| | | ACCGTATCGCGCACACCAAAGAAACTATGCTGTCTGA |
| | | CGGCCTGAACTCTCTCACGTACCAAGTTCTCGACGTA |
| | | CAGCGTTACCCGCTGTATACCCAGATCACCGTTGACA |
| | | TCGGAACCCCGTCTTGATGA |
| 53 | Chimeric hGalT | MIHTNLKKKFSLFILVFLLFAVICVWKKGSDYEALTLQAK |
| | polypeptide. Contains at | EFQMPKSQEKVAAAIGQSSGELRTGGARPPPPLGASS |
| | its 5′ end the polypeptide | QPRPGGDSSPVVDSGPGPASNLTSVPVPHTTALSLPAC |
| | for the CTS domain of the | PEESPLLVGPMLIEFNMPVDLELVAKQNPNVKMGGRYA |
| | rat hpha-2,6- | PRDCVSPHKVAIIIPFRNRQEHLKYWLYYLHPVLQRQQL |
| | sihyltransferase. | DYGIYVINQAGDTIFNRAKLLNVGFQEALKDYDYTCFVFS |
| | | DVDLIPMNDHNAYRCFSQPRHISVAMDKFGFSLPYVQY |
| | | FGGVSALSKQQFLTINGFPNNYWGWGGEDDDIFNRLVF |
| | | RGMSISRPNAVVGRCRMIRHSRDKKNEPNPQRFDRIAH |
| | | TKETMLSDGLNSLTYQVLDVQRYPLYTQITVDIGTPS |
| 54 | Coding sequence for 51 | ATGATTCACACGAACCTGAAGAAGAAGTTCAGCCTCT |
| | N-terminal amino acids | TCATCCTGGTTTTCCTGCTCTTCGCGGTAATCTGCGT |
| | from the cytoplasmic | TTGGAAGAAGGGTTCTGACTACGAAGCCCTCACCCTC |
| | transmembrane stem | CAGGCGAAGGAATTCCAGATGCCGAAGTCTCAGGAG |
| | region of rat alpha-2,6- | AAGGTT |
| | sialyltranferase (first 153 | |
| | nts from SEQ Id No: 17 | |
| | and SEQ Id No: 52) | |
| 55 | 51 N-terminal amino acids | MIHTNLKKKFSLFILVFLLFAVICVWKKGSDYEALTLQAK |
| | from the cytoplasmic | EFQMPKSQEKV |
| | transmembrane stem | |
| | region of rat alpha-2,6- | |
| | sialyltranferase | |
| 56 | MCS of PFC1484 and | ATTAATGGCGCGCCCTCGAG |
| | PFC1486 | |
| 57 | LB sequence plus | TGGCAGGATATATTGTGGTGTAAACAAATTGACGCTT |
| | AseI/AscI/SalI | AGACAACTTAATAACACATTGCGGACGTTTTTAATGTA |
| | multicloning site of | CTGATTAATGGCGCGCCGTCGAC |
| | PFC1488 | |
| 58 | AseI/AscI/SalI | ATTAATGGCGCGCCGTCGAC |
| | multicloning site of | |
| | PFC1488 | |
| 59 | CaMV 35S 5′ UTR. THIS | ACACGCTGAAATCACCAGTCTCTCTCTACAAATCTATC |
| | 41 NT SEQ IS 100% | |
| | (41/41) IDENTICAL WITH | |
| | Cauliflower mosaic virus, | |
| | complete genome TCT | |
| | Sequence ID: | |
| | NC_001497.2 | |
| 60 | Arabidopsis Act2 5′ UTR | ATTGTCTCGTTGTCCTCCTCACTTTCATCAGCCGTTTT |
| | sequence, including | GAATCTCCGGCGACTTGACAGAGAAGAACAAGGAAG |
| | intron. 100 % SEQ ID | AAGACTAAGAGAGAAAGTAAGAGATAATCCAGGAGAT |
| | (620/620) WITH | TCATTCTCCGTTTTGAATCTTCCTCAATCTCATCTTCTT |
| | Arabidopsis thaliana actin | CCGCTCTTTCTTTCCAAGGTAATAGGAACTTTCTGGAT |
| | 2 (ACT2) gene, complete | CTACTTTATTTGCTGGATCTCGATCTTGTTTTCTCAATT |
| | cds | TCCTTGAGATCTGGAATTCGTTTAATTTGGATCTGTGA |
| | Sequence ID: U41998.1 | ACCTCCACTAAATCTTTTGGTTTTACTAGAATCGATCT |
| | | AAGTTGACCGATCAGTTAGCTCGATTATAGCTACCAG |
| | | AATTTGGCTTGACCTTGATGGAGAGATCCATGTTCAT |
| | | GTTACCTGGGAAATGATTTGTATATGTGAATTGAAATC |
| | | TGAACTGTTGAAGTTAGATTGAATCTGAACACTGTCAA |
| | | TGTTAGATTGAATCTGAACACTGTTTAAGTTAGATGAA |
| | | GTTTGTGTATAGATTCTTCGAAACTTTAGGATTTGTAG |
| | | TGTCGTACGTTGAACAGAAAGCTATTTCTGATTCAATC |
| | | AGGGTTTATTTGACTGTATTGAACTCTTTTTGTGTGTT |
| | | TGCAGCTCATAAAAA |
| 61 | Arabidopsis Act2 5′ UTR | ATTGTCTCGTTGTCCTCCTCACTTTCATCAGCCGTTTT |
| | sequence, excluding | GAATCTCCGGCGACTTGACAGAGAAGAACAAGGAAG |
| | intron | AAGACTAAGAGAGAAAGTAAGAGATAATCCAGGAGAT |
| | | TCATTCTCCGTTTTGAATCTTCCTCAATCTCATCTTCTT |
| | | CCGCTCTTTCTTTCCAAGCTCATAAAAA |
| 62 | Arabidopsis Act8 5′ UTR | AGAATTGCCTCGTCGTCTTCAGCTTCATCGGCCGTTG |
| | sequence, including | CATTTCCCGGCGATAAGAGAGAGAAAGAGGAGAAAG |
| | intron. 1000/(623/623) | AGTGAGCCAGATCTTCATCGTCGTGGTTCTTGTTTCTT |
| | IDENTICAL TO | CCTCGATCTCTCGATCTTCTGCTTTTGCTTTTCCGATT |
| | Arabidopsis thaliana actin | AAGGTAATTAAAACCTCCGATCTACTTGTTCTTGTGTT |
| | 8 (ACT8) gene, complete | GGATCTCGATTACGATTTCTAAGTTACCTTCAAAAGTT |
| | cds | GTTTCCGATTTGATTTTGATTGGAATTTAGATCGGTCA |
| | Sequence ID: U42007.1 | AACTATTGGAAATTTTTGATCCTGGCACCGATTAGCTC |
| | | AACGATTCATGTTTGACTTGATCTTGCGTTGTATTTGA |
| | | AATCGATCCGGATCCTTTCGCTTCTTCTGTCAATAGG |
| | | AATCTGAAATTTGAAATGTTAGTTGAAGTTTGACTTCA |
| | | GATTCTGTTGATTTATTGACTGTAACATTTTGTCTTCC |
| | | GATGAGTATGGATTCGTTGAAATCTGCTTTCATTATGA |
| | | TTCTATTGATAGATACATCATACATTGAATTGAATCTA |
| | | CTCATGAATGAAAAGCCTGGTTTGATTAAGAAAGTGTT |
| | | TTCGGTTTTCTCGATCAAGATTCAGATCTTTATGTTTTT |
| | | GATTGCAGATCGTAGACC |
| 63 | Arabidopsis Act8 5′ UTR | AGAATTGCCTCGTCGTCTTCAGCTTCATCGGCCGTTG |
| | sequence, excluding | CATTTCCCGGCGATAAGAGAGAGAAAGAGGAGAAAG |
| | intron | AGTGAGCCAGATCTTCATCGTCGTGGTTCTTGTTTCTT |
| | | CCTCGATCTCTCGATCTTCTGCTTTTGCTTTTCCGATT |
| | | AAGATCGTAGACC |
| 64 | b12 Heavy chain aa seq. | MAKTNLFLFLIFSLLLSLSSAQVQLVQSGAEVKKPGASV |
| | The first 21 aa′s are the | KVSCQASGYRFSNFVIHWVRQAPGQRFEWMGWINPYN |
| | inventors′ version of | GNKEFSAKFQDRVTFTADTSANTAYMELRSLRSADTAV |
| | Arabidopsis basic | YYCARVGPYSWDDSPQDNYYMDVWGKGTTVIVSSAST |
| | chitinase signal peptide | KGPSVFPLAPSSKSTSGGTAALGCLVKDYFPEPVTVSW |
| | (the 2nd aa: Ala, was | NSGALTSGVHTFPAVLQSSGLYSLSSVVTVPSSSLGTQ |
| | added to make for a better | YTICNVNHKPSNTKVDKKAEPKSCDKTHTCPPCPAPELL |
| | Kozak box). | GGPSVFLFPPKPKDTLMISRTPEVTCVVVDVSHEDPEVK |
| | | FNWYVDGVEVHNAKTKPREEQYNSTYRVVSVLTVLHQ |
| | | DWLNGKEYKCKVSNKALPAPIEKTISKAKGQPREPQVY |
| | | TLPPSRDELTKNQVSLTCLVKGFYPSDIAVEWESNGQP |
| | | ENNYKTTPPVLDSDGSFFLYSKLTVDKSRWQQGNVFSC |
| | | SVMHEALHNHYTQKSLSLSPG |
| 65 | b12 Light chain aa seq. | MAKTNLFLFLIFSLLLSLSSAEIVLTQSPGTLSLSPGERAT |
| | The first 21 aa′s are the | FSCRSSHSIRSRRVAWYQHKPGQAPRLVIHGVSNRASG |
| | inventors version of | ISDRFSGSGSGTDFTLTITRVEPEDFALYYCQVYGASSY |
| | Arabidopsis basic | TFGQGTKLERKRTVAAPSVFIFPPSDEQLKSGTASVVCL |
| | chitinase signal peptide | LNNFYPREAKVQWKVDNALQSGNSQESVTEQDSKDST |
| | (the 2nd aa: Ala, was | YSLSSTLTLSKADYEKHKVYACEVTHQGLRSPVTKSFN |
| | added to make for a better | RGEC |
| | Kozak box). | |
| 66 | b12 Heavy chain nt seq | ATGGCTAAAACTAATCTGTTCCTTTTTCTTATTTTCTCT |
| | | TTACTCTTGTCCCTCAGTTCTGCTCAGGTTCAGTTAGT |
| | | TCAATCTGGCGCAGAGGTAAAGAAACCTGGAGCTAGT |
| | | GTGAAAGTTAGTTGCCAAGCTAGCGGATACAGGTTCT |
| | | CTAATTTTGTTATCCACTGGGTCCGTCAGGCTCCTGG |
| | | ACAGAGATTCGAATGGATGGGGTGGATTAATCCTTAC |
| | | AATGGAAACAAGGAGTTTAGCGCAAAATTTCAAGATA |
| | | GAGTTACTTTCACCGCCGATACAAGCGCTAATACAGC |
| | | CTATATGGAATTGAGATCATTACGATCTGCTGACACT |
| | | GCAGTCTATTACTGCGCCAGGGTCGGCCCATACTCCT |
| | | GGGATGACTCTCCTCAAGATAATTATTACATGGACGT |
| | | GTGGGGTAAGGGTACAACCGTCATAGTTTCATCTGCA |
| | | TCCACTAAGGGTCCTAGTGTTTTTCCTCTGGCACCAT |
| | | CTTCAAAGTCTACATCTGGCGGGACAGCTGCACTTGG |
| | | ATGCCTTGTGAAGGATTATTTTCCTGAACCAGTAACA |
| | | GTTAGCTGGAACTCCGGTGCTTTGACTTCAGGCGTTC |
| | | ATACTTTTCCTGCAGTACTTCAGAGTAGTGGATTGTAT |
| | | AGCTTGTCTAGCGTCGTTACTGTGCCTTCCTCTTCCC |
| | | TTGGGACACAAACATACATTTGCAATGTTAACCATAAA |
| | | CCATCTAATACTAAGGTTGACAAGAAAGCCGAGCCTA |
| | | AATCTTGTGATAAGACTCATACTTGTCCTCCATGTCCT |
| | | GCCCCTGAGTTGCTGGGAGGTCCATCCGTATTTCTCT |
| | | TCCCTCCAAAGCCAAAGGATACTTTGATGATTAGTCG |
| | | GACACCTGAAGTGACCTGTGTCGTGGTAGACGTTTCA |
| | | CATGAAGATCCAGAAGTTAAATTTAATTGGTACGTGG |
| | | ATGGAGTTGAGGTGCATAACGCTAAAACTAAGCCTAG |
| | | GGAAGAGCAATATAATTCAACCTACAGAGTTGTGTCA |
| | | GTCTTAACAGTGCTTCACCAAGATTGGTTAAACGGTA |
| | | AGGAATATAAGTGCAAAGTTTCAAATAAGGCTCTTCCT |
| | | GCTCCAATAGAAAAGACCATTTCTAAAGCTAAGGGAC |
| | | AACCTCGAGAACCTCAGGTATATACCCTCCCTCCAAG |
| | | TCGTGACGAATTGACAAAAAACCAGGTTTCTTTGACC |
| | | TGTTTGGTTAAAGGTTTTTATCCTAGTGATATCGCTGT |
| | | GGAGTGGGAGTCTAATGGTCAGCCTGAGAATAACTAT |
| | | AAGACTACTCCTCCAGTCCTCGATAGCGATGGTTCAT |
| | | TCTTTCTTTACTCTAAATTGACTGTAGATAAAAGCAGA |
| | | TGGCAACAGGGGAACGTGTTCTCATGTTCAGTTATGC |
| | | ACGAGGCACTGCACAATCATTATACTCAAAAGTCTCT |
| | | GTCATTGAGTCCTGGTTGA |
| 67 | b12 Light chain nt seq | ATGGCTAAGACTAACTTGTTTCTCTTTTTGATCTTCTC |
| | | ATTGCTTCTCTCCTTAAGCTCTGCTGAAATAGTTCTTA |
| | | CACAATCACCAGGAACTCTTAGTTTAAGTCCTGGCGA |
| | | GCGGGCTACCTTTTCTTGCCGAAGTTCCCACTCTATC |
| | | AGATCAAGACGAGTTGCATGGTATCAACACAAGCCAG |
| | | GACAAGCTCCAAGATTAGTGATTCATGGTGTAAGCAA |
| | | TAGGGCTTCTGGGATATCTGATCGTTTCTCAGGCTCA |
| | | GGTTCAGGTACAGACTTTACATTGACCATTACCAGGG |
| | | TTGAGCCAGAGGATTTCGCTCTTTACTATTGTCAGGTT |
| | | TATGGCGCAAGTTCTTACACTTTTGGGCAGGGAACCA |
| | | AACTGGAAAGGAAAAGGACTGTGGCTGCACCTTCTGT |
| | | GTTCATTTTTCCTCCATCCGATGAACAACTGAAGTCC |
| | | GGTACTGCCAGTGTTGTCTGTCTCTTGAATAACTTTTA |
| | | CCCAAGAGAGGCTAAGGTTCAGTGGAAAGTTGATAAC |
| | | GCCCTTCAATCTGGAAATAGCCAAGAAAGTGTAACAG |
| | | AGCAGGACTCTAAGGATTCCACATATTCTCTTTCTTCA |
| | | ACACTTACACTGAGCAAAGCAGATTACGAAAAACATA |
| | | AGGTCTATGCATGCGAAGTCACACATCAGGGACTTAG |
| | | ATCTCCTGTGACTAAGAGCTTCAATCGTGGTGAGTGT |
| | | TGA |
| 68 | PGV04 Heavy chain aa | MAKTNLFLFLIFSLLLSLSSAQVQLVQSGSGVKKPGASV |
| | seq. The first 21 aa′s are | RVSCWTSEDIFERTELIHWVRQAPGQGLEWIGWVKTVT |
| | the inventors version of | GAVNFGSPDFRQRVSLTRDRDLFTAHMDIRGLTQGDTA |
| | Arabidopsis basic | TYFCARQKFYTGGQGWYFDLWGRGTLIVVSSASTKGP |
| | chitinase signal peptide | SVFPLAPSSKSTSGGTAALGCLVKDYFPEPVTVSWNSG |
| | (the 2nd aa: Ala, was | ALTSGVHTFPAVLQSSGLYSLSSVVTVPSSSLGTQTYIC |
| | added to make for a better | NVNHKPSNTKVDKKVEPKSCDKTHTCPPCPAPELLGGP |
| | Kozak box). | SVFLFPPKPKDTLMISRTPEVTCVVVDVSHEDPEVKFNW |
| | | YVDGVEVHNAKTKPREEQYNSTYRVVSVLTVLHQDWL |
| | | NGKEYKCKVSNKALPAPIEKTISKAKGQPREPQVYTLPP |
| | | SREEMTKNQVSLTCLVKGFYPSDIAVEWESNGQPENNY |
| | | KTTPPVLDSDGSFFLYSKLTVDKSRWQQGNVFSCSVM |
| | | HEALHNHYTQKSLSLSPG |
| 69 | PGV04 Light chain aa | MAKTNLFLFLIFSLLLSLSSAEIVLTQSPGTLSLSPGETAS |
| | seq. The first 21 aa′s are | LSCTAASYGHMTWYQKKPGQPPKLLIFATSKRASGIPD |
| | the inventors′ version of | RFSGSQFGKQYTLTITRMEPEDFARYYCQQLEFFGQGT |
| | Arabidopsis basic | RLEIRRTVAAPSVFIFPPSDEQLKSGTASVVCLLNNFYPR |
| | chitinase signal peptide | EAKVQWKVDNALQSGNSQESVTEQDSKDSTYSLSSTL |
| | (the 2nd aa: Ala, was | TLSKADYEKHKVYACEVTHQGLSSPVTKSFNRGEC |
| | added to make for a better | |
| | Kozak box). | |
| 70 | PGV04 Heavy chain nt | ATGGCTAAAACAAATCTCTTTTTATTCTTGATTTTCTCC |
| | seq | CTTTTACTTTCCTTATCAAGCGCTCAAGTGCAACTCGT |
| | | TCAGTCTGGGTCTGGAGTTAAGAAACCTGGCGCCAG |
| | | TGTGAGGGTTTCATGTTGGACTTCCGAGGACATTTTT |
| | | GAACGTACTGAACTTATTCACTGGGTTAGACAAGCTC |
| | | CAGGTCAAGGGTTGGAGTGGATTGGCTGGGTCAAGA |
| | | CAGTAACTGGAGCTGTCAATTTTGGATCTCCAGATTT |
| | | CAGACAACGAGTGAGCTTGACACGGGATAGAGATCTT |
| | | TTTACAGCACATATGGATATAAGAGGTTTGACACAGG |
| | | GAGACACCGCTACATACTTTTGCGCAAGGCAGAAATT |
| | | CTATACTGGAGGTCAGGGCTGGTATTTCGATTTATGG |
| | | GGTAGGGGAACCCTGATCGTAGTATCAAGTGCTAGTA |
| | | CTAAGGGACCAAGCGTTTTTCCTTTAGCCCCAAGTTC |
| | | TAAGTCCACTAGTGGAGGTACCGCAGCTCTTGGTTGT |
| | | TTAGTCAAAGATTATTTCCCAGAGCCAGTTACCGTGA |
| | | GTTGGAACAGTGGTGCTTTGACTAGTGGAGTCCATAC |
| | | ATTCCCAGCTGTTTTGCAATCTAGTGGATTGTATTCAC |
| | | TCTCTAGTGTGGTTACCGTGCCATCATCAAGTTTAGG |
| | | AACACAAACATATATATGCAATGTGAATCATAAACCAA |
| | | GCAACACTAAAGTTGATAAGAAAGTGGAACCAAAGTC |
| | | ATGCGACAAAACACATACTTGTCCTCCATGCCCTGCA |
| | | CCTGAATTATTGGGAGGTCCTAGTGTTTTTTTATTTCC |
| | | ACCTAAACCAAAAGATACCCTTATGATTTCTAGGACAC |
| | | CAGAAGTTACTTGTGTCGTGGTCGATGTGTCCCATGA |
| | | AGATCCAGAAGTTAAATTCAATTGGTATGTGGATGGT |
| | | GTTGAAGTGCATAACGCTAAGACTAAGCCTAGGGAG |
| | | GAACAATATAATTCAACTTATAGAGTCGTTAGTGTCCT |
| | | TACTGTCCTCCACCAAGATTGGTTGAATGGAAAGGAG |
| | | TATAAATGCAAAGTCTCAAATAAGGCTCTCCCAGCAC |
| | | CTATCGAAAAAACCATATCCAAGGCCAAAGGACAACC |
| | | TAGAGAGCCTCAAGTTTATACACTTCCTCCATCTAGG |
| | | GAAGAAATGACAAAGAACCAAGTGAGCCTTACATGTC |
| | | TCGTTAAGGGTTTCTATCCTAGTGACATTGCCGTTGA |
| | | ATGGGAGAGTAATGGACAACCTGAGAACAATTATAAG |
| | | ACTACACCTCCAGTCTTGGATAGTGATGGTTCTTTCTT |
| | | TTTGTATTCTAAATTAACTGTTGACAAATCAAGATGGC |
| | | AACAGGGAAATGTTTTTTCATGTTCTGTCATGCACGA |
| | | GGCTCTTCACAATCATTATACTCAAAAATCACTTAGCC |
| | | TTAGCCCAGGATAA |
| 71 | PGV04 Light chain nt seq | ATGGCTAAAACAAATCTCTTTTTATTCTTGATTTTCTCC |
| | | CTTTTACTTTCCTTATCAAGCGCTGAGATAGTTTTAAC |
| | | ACAAAGCCCTGGCACCCTTTCTCTATCTCCAGGTGAA |
| | | ACTGCTTCGCTTTCATGCACTGCTGCCAGTTATGGAC |
| | | ATATGACATGGTATCAAAAGAAACCTGGACAGCCGCC |
| | | AAAGTTGCTTATCTTTGCAACCAGTAAACGTGCATCTG |
| | | GTATTCCCGATCGATTCTCCGGTTCACAGTTCGGCAA |
| | | GCAGTATACTCTCACGATTACTAGGATGGAACCTGAA |
| | | GACTTTGCTAGATACTACTGTCAACAGTTGGAGTTTTT |
| | | CGGGCAAGGAACAAGACTGGAGATCAGAAGGACCGT |
| | | GGCTGCACCAAGTGTGTTCATATTTCCTCCATCCGAT |
| | | GAACAATTGAAGAGTGGTACCGCAAGCGTCGTGTGTT |
| | | TATTGAATAACTTTTACCCAAGGGAAGCCAAAGTTCAA |
| | | TGGAAAGTTGATAATGCTCTCCAAAGTGGAAACTCAC |
| | | AAGAAAGTGTTACAGAGCAAGACTCAAAAGATTCCAC |
| | | TTATAGCTTATCATCTACACTTACACTCTCAAAAGCAG |
| | | ACTATGAAAAACACAAAGTCTACGCTTGCGAAGTCAC |
| | | TCATCAAGGACTTTCTTCACCAGTTACAAAGAGTTTCA |
| | | ATAGAGGAGAGTGTTAA |
| 72 | PGT121 Heavy chain aa | MAKTNLFLFLIFSLLLSLSSAQMQLQESGPGLVKPSETLS |
| | seq. The first 21 aa′s are | LTCSVSGASISDSYWSWIRRSPGKGLEWIGYVHKSGDT |
| | the inventors version of | NYSPSLKSRVNLSLDTSKNQVSLSLVAATAADSGKYYC |
| | Arabidopsis basic | ARTLHGRRIYGIVAFNEWFTYFYMDVWGNGTQVTVSSA |
| | chitinase signal peptide | STKGPSVFPLAPSSKSTSGGTAALGCLVKDYFPEPVTV |
| | (the 2nd aa: Ala, was | SWNSGALTSGVHTFPAVLQSSGLYSLSSVVTVPSSSLG |
| | added to make for a better | TQTYICNVNHKPSNTKVDKRVEPKSCDKTHTCPPCPAP |
| | Kozak box). | ELLGGPSVFLFPPKPKDTLMISRTPEVTCVVVDVSHEDP |
| | | EVKFNWYVDGVEVHNAKTKPREEQYNSTYRVVSVLTVL |
| | | HQDWLNGKEYKCKVSNKALPAPIEKTISKAKGQPREPQ |
| | | VYTLPPSREEMTKNQVSLTCLVKGFYPSDIAVEWESNG |
| | | QPENNYKTTPPVLDSDGSFFLYSKLTVDKSRWQQGNVF |
| | | SCSVMHEALHNHYTQKSLSLSPG |
| 73 | PGT121 Light chain aa | MAKTNLFLFLIFSLLLSLSSASDISVAPGETARISCGEKSL |
| | seq. The first 21 aa′s are | GSRAVQWYQHRAGQAPSLIIYNNQDRPSGIPERFSGSP |
| | the inventors′ version of | DSPFGTTATLTITSVEAGDEADYYCHIWDSRVPTKWVF |
| | Arabidopsis basic | GGGTTLTVLGQPKAAPSVFIFPPSDEQLKSGTASVVCLL |
| | chitinase signal peptide | NNFYPREAKVQWKVDNALQSGNSQESVTEQDSKDSTY |
| | (the 2nd aa: Ala, was GEC | SLSSTLTLSKADYEKHKVYACEVTHQGLSSPVTKSFNR |
| | added to make for a better | |
| | Kozak box). | |
| 74 | PGT121 Heavy chain nt | ATGGCTAAAACAAATCTCTTTTTATTCTTGATTTTCTCC |
| | seq | CTTTTACTTTCCTTATCAAGCGCTCAAATGCAGTTGCA |
| | | AGAATCTGGTCCTGGACTTGTTAAACCTAGCGAGACT |
| | | TTGTCATTAACATGCTCTGTCTCAGGTGCCAGTATTTC |
| | | TGATAGTTACTGGTCATGGATACGGAGAAGTCCAGGT |
| | | AAAGGACTCGAGTGGATTGGGTATGTGCACAAGTCTG |
| | | GTGATACAAATTACTCACCTAGTCTTAAGTCCAGAGTC |
| | | AATTTGAGCCTTGACACCTCCAAGAATCAAGTTTCTTT |
| | | GAGCTTAGTGGCTGCAACCGCTGCAGATTCTGGAAAA |
| | | TACTATTGTGCTAGGACTCTGCATGGGCGACGTATCT |
| | | ACGGCATTGTTGCTTTTAACGAATGGTTTACTTATTTC |
| | | TATATGGATGTTTGGGGCAACGGTACTCAAGTAACAG |
| | | TATCAAGTGCTAGTACTAAGGGACCAAGCGTTTTTCC |
| | | TTTAGCCCCAAGTTCTAAGTCCACTAGTGGAGGTACC |
| | | GCAGCTCTTGGTTGTTTAGTCAAAGATTATTTCCCAGA |
| | | GCCAGTTACCGTGAGTTGGAACAGTGGTGCTTTGACT |
| | | AGTGGAGTCCATACATTCCCAGCTGTTTTGCAATCTA |
| | | GTGGATTGTATTCACTCTCTAGTGTGGTTACCGTGCC |
| | | ATCATCAAGTTTAGGAACACAAACATATATATGCAATG |
| | | TGAATCATAAACCAAGCAACACTAAAGTTGATAAGAG |
| | | AGTGGAACCAAAGTCATGCGACAAAACACATACTTGT |
| | | CCTCCATGCCCTGCACCTGAATTATTGGGAGGTCCTA |
| | | GTGTTTTTTTATTTCCACCTAAACCAAAAGATACCCTT |
| | | ATGATTTCTAGGACACCAGAAGTTACTTGTGTCGTGG |
| | | TCGATGTGTCCCATGAAGATCCAGAAGTTAAATTCAAT |
| | | TGGTATGTGGATGGTGTTGAAGTGCATAACGCTAAGA |
| | | CTAAGCCTAGGGAGGAACAATATAATTCAACTTATAG |
| | | AGTCGTTAGTGTCCTTACTGTCCTCCACCAAGATTGG |
| | | TTGAATGGAAAGGAGTATAAATGCAAAGTCTCAAATAA |
| | | GGCTCTCCCAGCACCTATCGAAAAAACCATATCCAAG |
| | | GCCAAAGGACAACCTAGAGAGCCTCAAGTTTATACAC |
| | | TTCCTCCATCTAGGGAAGAAATGACAAAGAACCAAGT |
| | | GAGCCTTACATGTCTCGTTAAGGGTTTCTATCCTAGT |
| | | GACATTGCCGTTGAATGGGAGAGTAATGGACAACCTG |
| | | AGAACAATTATAAGACTACACCTCCAGTCTTGGATAGT |
| | | GATGGTTCTTTCTTTTTGTATTCTAAATTAACTGTTGAC |
| | | AAATCAAGATGGCAACAGGGAAATGTTTTTTCATGTTC |
| | | TGTCATGCACGAGGCTCTTCACAATCATTATACTCAAA |
| | | AATCACTTAGCCTTAGCCCAGGATAA |
| 75 | PGT121 Light chain nt | ATGGCTAAAACAAATCTCTTTTTATTCTTGATTTTCTCC |
| | seq | CTTTTACTTTCCTTATCAAGCGCTTCTGACATATCCGT |
| | | CGCACCTGGAGAGACAGCTCGTATCAGCTGCGGTGA |
| | | AAAATCATTAGGGAGCAGAGCCGTTCAATGGTATCAA |
| | | CATAGGGCTGGTCAGGCACCATCTTTGATCATTTACA |
| | | ACAATCAAGATCGGCCATCAGGTATTCCTGAACGATT |
| | | TTCTGGTTCTCCTGATTCACCATTTGGAACAACTGCTA |
| | | CCCTCACTATTACAAGTGTTGAAGCTGGGGACGAGG |
| | | CTGATTACTATTGTCACATATGGGATAGTAGAGTGCC |
| | | AACCAAGTGGGTATTCGGCGGAGGCACTACTCTTACT |
| | | GTTCTGGGACAGCCAAAGGCTGCACCAAGTGTGTTC |
| | | ATATTTCCTCCATCCGATGAACAATTGAAGAGTGGTA |
| | | CCGCAAGCGTCGTGTGTTTATTGAATAACTTTTACCCA |
| | | AGGGAAGCCAAAGTTCAATGGAAAGTTGATAATGCTC |
| | | TCCAAAGTGGAAACTCACAAGAAAGTGTTACAGAGCA |
| | | AGACTCAAAAGATTCCACTTATAGCTTATCATCTACAC |
| | | TTACACTCTCAAAAGCAGACTATGAAAAACACAAAGTC |
| | | TACGCTTGCGAAGTCACTCATCAAGGACTTTCTTCAC |
| | | CAGTTACAAAGAGTTTCAATAGAGGAGAGTGTTAA |
| 76 | LB region of PF01403 | TGGCAGGATATATTGTGGTGTAAACAAATTGACGCTT |
| | and PF01405. 78 nt; first | AGACAACTTAATAACACATTGCGGACGTTTTTAATGTA |
| | 25 nt are LB sequence = | CTG |
| | seq id 14; last 53 nt are LB | |
| | associated seq | |
| 77 | MCS of PF01403 and | ATTAATGGCGCGCCGTCGAC |
| | PF01405. AseI, AscI, | |
| | SalI restriction sites. | |
| 78 | Reverse complement of | GATCTAGTAACATAGATGACACCGCGCGCGATAATTT |
| | nos terminator = | ATCCTAGTTTGCGCGCTATATTTTGTTTTCTATCGCGT |
| | terminator sequence of | ATTAAATGTATAATTGCGGGACTCTAATCATAAAAACC |
| | nopaline synthase gene. | CATCTCATAAATAACGTCATGCATTACATGTTAATTATT |
| | PF01403 and PF01405. | ACATGCTTAACGTAATTCAACAGAAATTATATGATAAT |
| | First 253 nt have 100% | CATCGCAAGACCGGCAACAGGATTCAATCTTAAGAAA |
| | identity with 253 nt of | CTTTATTGCCAAATGTTTGAACGATCG |
| | GenBank accession | |
| | Sequence ID: | |
| | gi\|159141737\|AE007871. | |
| | 2; note that a ″C″ is tacked | |
| | on at the end as this is a | |
| | 254 nt seq, and that the is | |
| | a cloning artifact that | |
| | resides between nosT | |
| | and the Pat gene stop | |
| | codon | |
| | Agrobacterium | |
| | tumefaciens str. 058 | |
| | plasmid Ti, complete | |
| | sequence | |
| 79 | PFC synthetic seq: PAT | TCAGATCTCAGTAACTGGAAGAACTGGTCTTGGTGGA |
| | (phosphinothricin | ACTGGAAGTGAGAAATCGAGCTGCCAGAATCCAACAT |
| | acetyltransferase) coding | CATGCCAATTTCCGTGCTTAAAACCAGCAGCTCTAAG |
| | sequence; reverse | CATTCCTCTTGGAGCATATCCAAGAGCCTCATGCATT |
| | complement. PFC 1403 | CTAACAGATGGATCGTTTGGGAGTCCAATCACAGCAA |
| | and PF01405. 100% | CAACAGACTTGAATCCTTGAGCCTCAAGAGACTTGAG |
| | identity with 183 aa′s of | AAGGTGAGTGTAAAGAGTAGATCCAAGTCCAGTCCTC |
| | GenBank Sequence ID: | TGATGTCTTGGTGAAACGTAAACAGTGGACTCAGCAG |
| | gi\|114833\|P16426.1 | TCCAATCATAAGCATTCCTAGCCTTCCATGGTCCAGC |
| | RecName: | ATAAGCAATTCCAGCAACTTCACCATCAACTTCAGCAA |
| | Full = Phosphinothricin | CAAGCCATGGATACCTTTCCCTGAGCCTAACAAGATC |
| | N-acetyltransferase; | ATCAGTCCATTCTTGTGGCTCTTGTGGTTCAGTCCTAA |
| | Short = PPT N- | AGTTCACAGTGGAAGTCTCAATGTAGTGGTTCACAAT |
| | acetyltransferase; | AGTGCACACAGCTGGCATATCAGCTTCAGTAGCCCTT |
| | AltName: | CTAATATCAGCTGGCCTTCTTTCTGGAGACAT |
| | Full = Phosphinothricin- | |
| | resistance protein | |
| 80 | BamHI cloning site of | GGATCC |
| | PF01403 and PF01405 | |
| 81 | reverse complement of | TGCAGATTATTTGGATTGAGAGTGAATATGAGACTCTA |
| | nos promoter = promoter | ATTGGATACCGAGGGGAATTTATGGAACGTCAGTGGA |
| | of nopaline synthase | GCATTTTTGACAAGAAATATTTGCTAGTGATAGTGACC |
| | gene. PF01403 and | TTAGGCGACTTTTGAACGCGCAATAATGGTTTCTGAC |
| | PF01405. 99% identity | GTATGTGCTTAGCTCATTAAACTCCAGAAACCCGCGG |
| | (207/208) with GenBank | CTCAGTGGCTCCTTCAACGT |
| | accession AE007871.2 | |
| | Agrobacterium | |
| | tumefaciens str. 058 | |
| | plasmid Ti, complete | |
| | sequence | |
| 82 | MCS of PF01403 and | GGGCCCGGCGCCGCTAGC |
| | PF01405. AseI, AscI, | |
| | SalI restriction sites. | |
| 83 | N. benthamiana repeat | |
| | ″B″ consensus sequence. | TATTCCCTTGTTCTACAGGTGGGCGCCTGATTACCAA |
| | PF01403 and PF01405. | AACTTGCAACTTGAAAA |
| 84 | Cloning site, SpeI. | ACTAGT |
| | PF01403 and PF01405. | |
| 85 | Reverse complement of | AAGATAACGAAACTATAATAAAATCTTTCAATCACTGT |
| | rbcT = terminator of | CTGACATGTTATTTTATAAAAAAATTTGTGGATGTTATT |
| | rubisco gene. PF01403 | ATGCGGTTGTAGCATTCCTTATTTGAAAATGTTGAAAA |
| | and PF01405 100% | TGCCTTCATGTAGGAAGTATATGGAAAGGTGTACTTTT |
| | identity with 349 nt of | TGGCTAGGTAATTTATTCAGTACGATGTACTGTAGTTT |
| | GenBank accession | ATTTTGTACTTTTATTTAACATTATTTCCCCCTAATATA |
| | AY163904.1 | ACAAGAAGAAAATGTCATACATACAATTAATTAAAGGT |
| | Chrysanthemum x | TTGTAAAAAAAAGATTGCAAATATGAAACGACAATTTA |
| | morifoliumri bulose-1, 5- | TTATTTATATATGATATCTCTTACATTTCATTATCGATC |
| | bisphosphate carboxylase | CGGA |
| | small subunit gene, | |
| | complete cds; nuclear | |
| | gene for chloroplast | |
| | product | |
| 86 | Cloning site, XhoI. | CTCGAG |
| | PF01403 and PF01405. | |
| 87 | PFC synthetic seq: hGalT; | TCATCAAGACGGGGTTCCGATGTCAACGGTGATCTG |
| | n.b.reverse complement. | GGTATACAGCGGGTAACGCTGTACGTCGAGAACTTG |
| | PF01403 and PF01405. | GTACGTGAGAGAGTTCAGGCCGTCAGACAGCATAGT |
| | | TTCTTTGGTGTGCGCGATACGGTCAAAGCGCTGCGG |
| | | GTTCGGCTCGTTCTTCTTGTCACGGCTGTGACGGATC |
| | | ATACGGCAGCGGCCCACGACCGCATTCGGACGGCTG |
| | | ATAGACATACCGCGGAATACGAGGCGGTTGAAGATAT |
| | | CATCGTCTTCACCACCCCAACCCCAGTAATTGTTCGG |
| | | GAAACCGTTGATCGTCAGGAATTGCTGCTTAGAGAGG |
| | | GCAGACACGCCACCGAAGTACTGTACATACGGGAGA |
| | | GAGAAACCGAATTTGTCCATCGCAACAGAGATGTGAC |
| | | GTGGTTGAGAAAAGCAACGGTAGGCGTTGTGATCATT |
| | | CATCGGGATCAGGTCAACGTCAGAGAAAACGAAGCA |
| | | GGTGTAGTCGTAATCCTTGAGCGCCTCCTGGAAACCC |
| | | ACGTTCAGCAGTTTAGCGCGGTTAAAGATGGTGTCAC |
| | | CCGCCTGGTTGATAACGTAGATACCGTAGTCGAGCTG |
| | | CTGACGCTGCAGAACCGGGTGCAGGTAGTACAGCCA |
| | | GTATTTCAGGTGCTCTTGACGGTTACGGAAAGGAATG |
| | | ATGATGGCCACCTTGTGCGGGGAAACGCAATCACGA |
| | | GGGGCGTAGCGACCACCCATCTTTACGTTCGGGTTC |
| | | TGCTTCGCAACGAGTTCCAGGTCTACCGGCATGTTGA |
| | | ATTCGATGAGCATAGGGCCTACCAGCAGCGGAGATT |
| | | CTTCCGGGCACGCCGGCAGGCTGAGCGCGGTGGTA |
| | | TGTGGAACCGGAACAGACGTCAGGTTAGAAGCTGGA |
| | | CCTGGGCCAGAATCCACAACCGGAGAGCTGTCGCCA |
| | | CCCGGACGCGGCTGGCTAGATGCACCCAGCGGCGG |
| | | TGGAGGACGAGCACCACCGGTACGGAGTTCACCAGA |
| | | GGACTGACCGATGGCTGCGGC |
| 88 | PFC synthetic seq: CTS; | AACCTTCTCCTGAGACTTCGGCATCTGGAATTCCTTC |
| | n.b. reverse complement. | GCCTGGAGGGTGAGGGCTTCGTAGTCAGAACCCTTC |
| | PF01403 and PF01405. | TTCCAAACGCAGATTACCGCGAAGAGCAGGAAAACCA |
| | | GGATGAAGAGGCTGAACTTCTTCTTCAGGTTCGTGTG |
| | | AATCAT |
| 89 | Cloning site, HindIII. | AAGCTT |
| | PF01403 and PF01405. | |
| 90 | RB sequence. PF01403 | TGACAGGATATATTGGCGGGTAAAC |
| | and PF01405. | |
| 91 | RB; n.b. that this includes | TGACAGGATATATTGGCGGGTAAACCTAAGAGAAAAG |
| | the 25 nt in the RB | AGCGTTTATTAGAATAATCGGATATTTAAAAGGGCGT |
| | sequence (SEQ ID NO: | GAAAAGGTTTATCCGTTCGTCCATTTGTATGTGCATG |
| | 90); Agrobacterium | CCAACCACAGGGTTCCCC |
| | tumefaciens Ti plasmid | |
| | pTiC58 T-DNA region | |
| 92 | Synthetic DNA insertion in | AAGATAACGAAACTATAATAAAATCTTTCAATCACTGT |
| | PF01403; includes (all | CTGACATGTTATTTTATAAAAAAATTTGTGGATGTTATT |
| | reverse complement): rbc | ATGCGGTTGTAGCATTCCTTATTTGAAAATGTTGAAAA |
| | Terminator, LmSTT3D | TGCCTTCATGTAGGAAGTATATGGAAAGGTGTACTTTT |
| | coding sequence and 35S | TGGCTAGGTAATTTATTCAGTACGATGTACTGTAGTTT |
| | basal promoter | ATTTTGTACTTTTATTTAACATTATTTCCCCCTAATATA |
| | | ACAAGAAGAAAATGTCATACATACAATTAATTAAAGGT |
| | | TTGTAAAAAAAAGATTGCAAATATGAAACGACAATTTA |
| | | TTATTTATATATGATATCTCTTACATTTCATTATCGATC |
| | | CGGAGGTACCTCATCACCTTCTATTCTCAGACTCACG |
| | | CATCCTCCGCATGTACTCCTCTTGATAAGACCCCACG |
| | | TTGTTCTTCCGATCAGCGTTGGTAACCTGATCGAACG |
| | | GCACCCTATGAGCCAGCATCTCTTGAATTTCTTTGGC |
| | | CGGAGGGTACTGACCAGGACAAATCCAAGAACCAGG |
| | | AGGATGGCACACCCTATTAGCAGGATCTGCAACCCAC |
| | | TTCTTGCTCTCGGCGCTCACATTCATCACCTTGAAGA |
| | | TCCTCACCAGGCCGTACTTAGAGCTGTACACCTCTTG |
| | | GAACAAGCTCGGGTTCACCTTAACACCTTTCCGCTTA |
| | | CCAGCCTCGTGAAGGTTGTAAAGAAGAGAAGCCCGC |
| | | ATCATAGGAGTAGGCCGAGAGTAATCATTCCGGTGGA |
| | | AACCGAACTGCTGGCAAAGAGGATCATCAGGGCAGA |
| | | TATCGTGGTACACAGAGTTGCCGATCCTAGCCATGTG |
| | | AGGAGACTTCATAAGATCGCCAGACTGACCAGCCCAA |
| | | ATCAGCACGTAATCAGCCATGTGCCTAACAAGAGAGT |
| | | GAGCCTCGACAACAGGGCTAGTAAGCATCTTACCGAT |
| | | GGTGGCAATGTGCTCGTGGTTCCAAGTATTACCATCA |
| | | GCCAGAGAGGTCCTGTTGCCAATACCGGTAATCTGGT |
| | | AGCCGTAATCCCACCAAGCGAGAACTCTAGCATCCTC |
| | | AGGAGTAGAATCCCTCAGCCACTCGTAAGCCTTCAGG |
| | | TAATCATCCACCAGCAGGTTCATAGGCTTGCCAGTAG |
| | | CACGATTCTGCACAACAGCAGCGAACACAATCATCGG |
| | | GTTGCTTGACTGCTCAGCGAACTTAGTGCTGTGGGAA |
| | | GCGAATTCGGAGGAGAAGAAAGAAACGGCGGTGGTA |
| | | GTCACAAGAGCCCACATTGCAATAGAAAGCACCATAC |
| | | GGTGACCCCAAGCCAGAGAAGAACCAGCAAACACAT |
| | | CACAGAAAGCCCGAGCGGTAGTAGCATTCTTAGCGT |
| | | CATCCCTACCAGAACCTTTACCAGCACCTCTCTGATG |
| | | CCTCTGAGCCTGCTTTTGCTGCTTTTTGGCCTTGGTA |
| | | GCATCAGAATCCCAGAAAGACAACTGCACGGCAGCTT |
| | | CAAGAATGGTGCCCACGAAAATACCGGTGCTAAGACA |
| | | AGCAGCAGGTCCAGAAAGAAGCAGGAGCCTAGCCAT |
| | | CCTAGTAGAGAAGTAGTACACGGCGCCAGAGTTCAG |
| | | AAGCCAGAACACCTTAGAAGGGCTGTAGTGCACGAA |
| | | GGTAGACACAGCCAACACAATAGAACCCAGACCCCA |
| | | AGTCACACCGCACACATGAAGAAAAGCCCACATAGCC |
| | | TCTGGAGAAGCAGGCTGATGCTCAGCAACAGAATCAA |
| | | CCAGAGGGTTACCAGTCCTGGTATGCTCAACGAACAA |
| | | GGCTCTCACCCTAACAGACAAAGGACCGAAGTAACC |
| | | GGTAGGAGCAAGCACAGAAATAGCAAGAGCTGCAAC |
| | | GCCAGCCATAACGGAGAACACCCTCACTCTGATCTTG |
| | | AAGTTAGC |

### Example 8

Using Mendelian Genetics to Determine how Many T-DNA Loci are Inserted into the Genome of T₀ Plant 1403-25

It is desirable to develop a homogeneous stable transgenic plant line from primary transgenic plant 1403-25.

Basta® resistance segregation was tested to determine how many PFC1403 T-DNA loci were inserted into the genome of T₀ plant 1403-25. To do this, 148 T1 seed from self-pollinated T₀ plant 1403-25 were plated on sterile agar plates containing 10 mg/L phosphothrinicin (Basta®). Of these 148 seed, 20 did not germinate; however, 128 seeds germinated and of the plantlets that grew from these 118 were determined to be resistant to Basta® while 10 were not.

If a single T-DNA locus was inserted into the genome of T₀ plant 1403-25 then according to laws of Mendelian inheritance one would expect that a dominant Basta®-resistant trait would be inherited in a ratio of 3 Basta®-resistant plants to 1 Basta®-susceptible plant; i.e., of 128 T1 seeds that germinated one would expect that approximately 96 plants (75%) would be resistant to Basta® and that approximately 32 plants (25%) would be susceptible to Basta®.

Testing 118 resistant plants and 10 susceptible plants for a segregation ratio of 3:1 resulted in a chi-square statistic of 13.7855 with a p-value of 0.000205. This result is significant at p<0.05 and as such the low p-value implies that the null hypothesis is rejected; i.e., a 3:1 segregation ratio of R:S T1 plants cannot explain the inheritance of genes conferring Basta® resistance from a self-pollinated T₀ transgenic plant.

If two independent T-DNA loci were inserted into the genome of T₀ plant 1403-25 then according to Mendelian inheritance one would expect that a dominant Basta®-resistant trait would be inherited in a ratio of 15 Basta®-resistant plants to 1 Basta®-susceptible plant; i.e., of 128 T1 seeds that germinated one would expect that approximately 120 plants (93.75%) would be resistant to Basta® and that approximately 8 plants (6.25%) would be susceptible to Basta®.

Testing 118 resistant plants and 10 susceptible plants for a segregation ratio of 15:1 results in a chi-square statistic of 0.239 with a p-value of 0.624908. This result is not significant at p<0.01. This high p-value implies that the null hypothesis cannot be rejected; i.e., a 15:1 segregation ratio of R:S T1 plants is best explained by a model of inheritance from a self-pollinated T0 plant containing two independent (unlinked) T-DNA insertions (loci), each with a dominant allele that confers Basta® resistance.

Selecting a Homozygous Transgenic Plant Line from T1 Plants

Developing a homozygous plant line from a T0 plant that contains 2 independent T-DNA loci involves more work that from a T0 plant that contains only 1 T-DNA locus. This is because according to laws of Mendelian inheritance for a dominant, single-locus trait one would expect that 1 in 4 T1 plants from self-pollinated T0 plant 1403-25 would be homozygous for the transgene. As T0 plant 1403-25 has 2 independent T-DNA insertions, one would expect that 1 in 16 T1 plants from self-pollinated T0 plant 1403-25 would be homozygous at both transgene loci.

However, the potential contributions to the GaIT phenotype that either of these 2 independent transgene loci provide should be considered. Of the 20 T0 plants that were assessed for GaIT activity as shown in FIG. 14 only 1 plant (i.e., T0 plant 1403-25) was determined to have such activity. If these 20 T0 plants had only 1 T-DNA insertion, and since only 1 of these 20 insertions was determined to have GaIT activity, it is therefore reasonable to expect that for a plant such as T0 plant 1403-25 which has GaIT activity and 2 independent T-DNA insertions that only 1 of the 2 T-DNA insertions provides GaIT activity. (Many T0 plants are expected to have only single T-DNA insertions, while a few are expected to have more than 1 insertion; therefore, 20 T0 plants having only 1 T-DNA insertion each is a very conservative estimate for this argument).

Therefore, to develop a homozygous transgenic line for GaIT activity, it may be desirable to (i) breed the active GaIT T-DNA locus to homozygosity and (ii) breed the inactive GaIT T-DNA locus out of the line that is to be developed.

To do this, sufficient seed produced by self-pollinated T0 plant 1403-25 were germinated to raise 56 T1 plants to maturity. Likewise, each of these T1 plants were self-pollinated, and their T2 seedlots were harvested. Each of these 56 T2 seedlots originated from T1 plants that were numbered 1403-25-1 through 1403-25-56. Also likewise to the T1 seedlot produced by T0 plant 1403-25, sufficient seed from each of these T2 seedlots were subjected to Basta®-resistance segregation analysis with a goal of identifying T2 seedlots that were 100% Basta®-resistant; however, because we did not want to overlook any T1 plant line that had potential value due to biological variation and difficulties scoring this bioassay with absolute certainty as mentioned above, we chose to study further those T2 seedlots that had >95% resistance to Basta®. It was found that among the 56 T2 seedlots were 11 such seedlots that had >95% resistance to Basta®. The following Table 13 gives the Basta® resistant:susceptible ratios among T2 progeny of T1 plants numbered 1403-25-xx [where xx ranges from 01 through 56] that were chosen for further study.

TABLE 13
Basta ® resistant:susceptible ratios among T2 progeny selected for
further study from self-pollinated T1 plants numbered 1403-25-xx,
where xx ranges from 01 to 56.

| T1 plant | Resistant | Susceptible | % resistant |
|---|---|---|---|
| 1403-25-01 | 95 | 4 | 96% |
| 1403-25-07 | 99 | 1 | 99% |
| 1403-25-11 | 97 | 0 | 100% |
| 1403-25-16 | 98 | 1 | 99% |
| 1403-25-19 | 98 | 0 | 100% |
| 1403-25-21 | 99 | 0 | 100% |
| 1403-25-24 | 87 | 1 | 99% |
| 1403-25-25 | 96 | 2 | 98% |
| 1403-25-39 | 89 | 0 | 100% |
| 1403-25-54 | 50 | 1 | 98% |
| 1403-25-55 | 94 | 0 | 100% |

### Determining Whether T2 Plants Having >95% Basta® Resistance Express Gait Activity

To determine whether the T2 plants having >95% Basta® resistance express GaIT activity, 8 T2 plants per T1 plant line were agroinfiltrated with trastuzumab vector PFC0058. Also, as controls, (i) KDFX plants were infiltrated with vector PFC0058 to provide a negative control for GaIT activity, and (ii) sample from T1 plants derived from T0 plant 1403-25 that was positive for GaIT activity in FIG. 14 was applied as a positive control for GaIT activity. As was done above for the screen to identify GaIT expression in T1 plants resulting from self-pollinated primary transgenic T0 plants (FIG. 14), trastuzumab antibody was purified using Protein G and 3 μg trastuzumab per sample was analysed by 10% SDS-PAGE under reducing conditions with Coomassie Blue gel staining, and 1.2 μg trastuzumab per sample was analyzed by western blot followed by RCA probing to identify T1 plant lines with GaIT activity

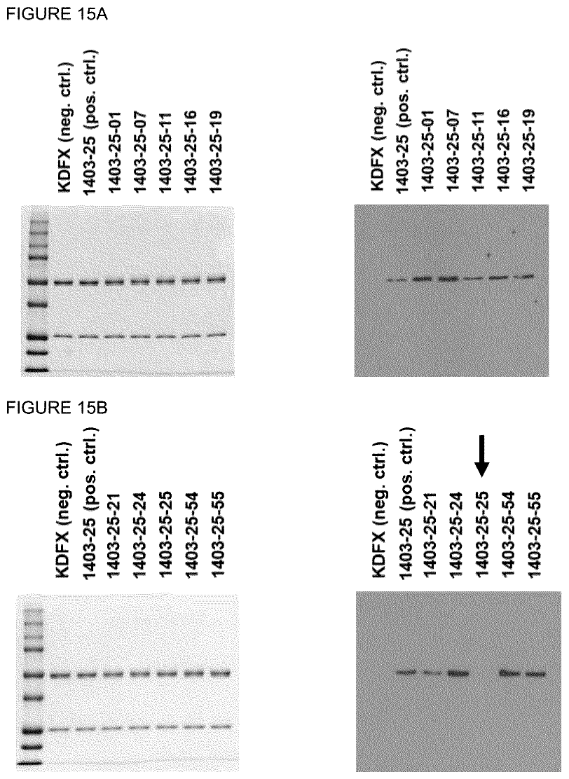

The panels in FIG. 15 below show the results of these analyses. As can be seen from the 2 Coomassie blue-stained gels on the left of the 2 panels below that trastuzumab from all samples was applied equivalently to each gel. As can also be seen in FIG. 15, trastuzumab samples equivalently loaded onto gels and transferred to western blots were probed with RCA lectin for GaIT activity: the KDFX negative control showed no GaIT activity, as expected; the 1403-25 positive control showed GaIT activity, as expected from the results of the experiment of FIG. 14; and 9 of the 10 samples from the T1 lines showed GaIT activity. (Note that plantline 1403-25-39 was not included in this analysis; it was analyzed in another experiment for which data are not shown).

It is important to note that T1 plantline 1403-25-25 did not show any GaIT activity among its T2 progeny (highlighted by black arrow in 2^{nd} panel below of FIG. 15). This result, combined with the fact that the T2 progeny from self-pollinated T1 plant 1403-25-25 could be considered to effectively have 100% Basta®-resistance, suggests that T1 plant 1403-25-25 is homozygous for an inactive GaIT insertion and likely homozygous null (i.e., no T-DNA insertion) at the locus that contains the active GaIT insertion in T0 plant 1403-25.

### Assessment of Glycans on Trastuzumab Antibody Produced by T2 Progeny of Self-Pollinated T1 Plants Chosen for Development of a Homozygous Stable Transgenic Gait Plant Line

The trastuzumab antibody samples that were purified from the T2 sibling plants and analyzed by RCA-probing of western blots as shown in the panels of FIG. 15 were also assessed for amounts of glycan species as was done for the data provided in Tables 3, 5, 7 and 9 above. Table 14 below shows results of these analyses.

TABLE 14
Glycan species quantifications on trastuzumab antibody purified from T2 sibling plant pools from self-pollinated
T1 transgenic plant 1403-25. Some glycan species have been pooled (e.g., mannosylated glycans) to simplify the table.

| T1 plant # | 1403-25-01 | 1403-25-07 | 1403-25-11 | 1403-25-16 | 1403-25-19 | 1403-25-21 | 1403-25-24 | 1403-25-25 | 1403-25-55 |
|---|---|---|---|---|---|---|---|---|---|
| Glycans | 46.417 | 29.874 | 49.739 | 34.473 | 32.675 | 54.839 | 31.936 | 79.722 | 30.033 |
| GnGn | | | | | | | | | |
| AM | 2.201 | 4.827 | 4.152 | 3.82 | 4.268 | 2.488 | 3.859 | | 6.421 |
| AGn | 22.885 | 31.133 | 19.005 | 31.795 | 31.617 | 20.186 | 33.375 | | 27.84 |
| AA | 3.226 | 6.231 | 3.215 | 6.521 | 6.393 | 3.288 | 7.808 | | 5.674 |
| Man5-9 | 24.308 | 21.725 | 19.767 | 19.683 | 20.674 | 17.453 | 19.126 | 14.087 | 20.196 |
| Minor | 0.964 | 6.211 | 4.123 | 3.707 | 4.373 | 1.747 | 3.895 | 6.191 | 9.836 |
| glycans | | | | | | | | | |
| Total | 100.001 | 100.001 | 100.001 | 99.999 | 100 | 100.001 | 99.999 | 100 | 100 |

As can be seen from Table 14, T2 plants from self-pollinated T1 plant 1403-25-25 produced glycans on trastuzumab antibody that were completely lacking galactosylation (AM, AA, AGn). This further confirms that this T1 line lacks GaIT activity; combined with the fact that these T2 plants are Basta®-resistant and thus contain T-DNA insertions we can be further assured that only 1 of the 2 T-DNA loci in T0 plant 1403-25 has GaIT activity.

Also, as can be seen from Table 14, each of the 10 other lines of T2 sibling plant pools were shown to have appreciable GaIT activities. T2 sibling plant pools from T1 plant lines 1403-25-01, -11 and -21 showed GaIT activities that resulted in less than 30% total glycan species galactosylation (i.e., AM, AGn and AA glycan species), while T2 sibling plant pools from T1 plant lines 1403-25-07, -16, -19, -24, and -55 showed GaIT activities that resulted in more than approximately 40% total glycan galactosylation

### DISCUSSION

In order to breed and select for a stable transgenic plant line that (i) expresses GaIT activity, (ii) is homozygous at the active GaIT T-DNA locus and (iii) is lacking a T-DNA insertion at the inactive GaIT locus (i.e., homozygous null at that locus), whole-genome sequencing is used. To do this, T2 plants are propagated maturity from each of the 11 T1 lines that were chosen for further study. For each of these lines, a single T2 plant was chosen (i) for a leaf tissue sample, from which genomic DNA was prepared for whole-genome sequencing and (ii) for self-pollination to provide a T3 seed lot for plant line maintenance and propagation of further generations.

T1 plant lines 1403-25-19 and 1403-25-55 were chosen for whole-genome sequencing because T2 sibling plant pools from both of these self-pollinated T1 plants showed both bona fide 100% Basta® resistance and higher (approximately 40%) total glycan species galactosylation, It is expected that these 2 plant lines should be homozygous at the single T-DNA locus that is provides GaIT activity.

Thus, it is expected to find the PFC1403 T-DNA sequence associated with N. benthamiana genomic sequences at a single locus.

However, it is possible that either of these 2 T2 plant DNA samples have PFC1403 T-DNA sequence associated with another N. benthamiana genomic locus. This second N. benthamiana genomic locus would be identifiable as a different genomic DNA sequence and the T-DNA inserted there would not provide GaIT activity (i.e., the GaIT inactive locus). To aid in the identification of such a locus, DNA from T1 plant line 1403-25-25 was also chosen for whole-genome sequencing because it should lack T-DNA insertions at the active GaIT T-DNA locus. Its PFC1403 T-DNA sequence would be associated with unique N. benthamiana genomic DNA sequences that would therefore be useful for identification of the GaIT inactive locus.

Should T2 DNA samples from either T1 plant 1403-25-19 or T1 plant 1403-25-55 have PFC1403 T-DNA sequence associated with the inactive GaIT locus, it would be desirable to select a plant from either its T2 siblings or from its T3 offspring that entirely lacks PFC1403 T-DNA sequence associated with the inactive GaIT locus. To aid in doing this, so as to avoid selection relying upon another round of whole-genome sequence and bioinformatic analyses, diagnostic PCR reactions could be developed using unique N. benthamiana genomic sequence flanking both the GaIT active T-DNA insertion and the GaIT inactive T-DNA insertion. These unique flanking genomic sequences would be used for the development of oligonucleotide primers that would allow for the specific amplification of unique DNA products that would differ in size for either of the 2 T-DNA insertion loci. These diagnostic PCR reactions would therefore be used to select plants that are (i) homozygous at the active GaIT locus and (ii) homozygous-null at the inactive GaIT locus.

Should it be necessary to breed the inactive GaIT T-DNA out of either of the plant lines being derived from T1 transgenic plants 1403-25-19 or 1403-25-55, either at the T2 generation or the T3 generation, once the PCR test indicates which plant(s) should be selected for propagation of a homozygous GaIT plant line with GaIT activity, (i) whole-genome sequence analysis would be performed to verify zygosity and genotypes at the GaIT active and GaIT inactive loci, and (ii) that or those plant(s) would be self-pollinated for production of next-generation seed for continual propagation of the desired plant line. Lastly, next-generation plants would be propagated and treated for expression of trastuzumab antibody for verification of sustained GaIT activity by this plant line.

It has been demonstrated that the GaIT lines described above are compatible with vectors expressing trastuzumab. In addition, it has been shown that functionality of exogenous chimeric human alpha-1,6-fucosyltransferase (FucT) and Leishmania major oligosaccharyltransferase (STT3D) is unaffected in the 1403-25-XX seed lines when co-introduced with the trastuzumab vector 0058.

A sufficient number of primary transgenic plants were produced and screened to allow for identification of a single plant line that could perform galactosylation of a target protein of interest. Because the PFC1403 vector was entirely lacking promoter and 5′UTR sequences, it was anticipated that the frequency of selecting transgenic plant lines with GaIT activity would be low. Without being bound by theory, GaIT activity has possibly resulted due to insertion of the PFC1403 T-DNA into a region of the N. benthamiana genome that could support weak but sufficient expression of GaIT enzyme.

A stable transgenic, homozygous line as described herein can be crossed with other plant lines. For example, the stable transgenic line could be crossed with a KDFX plant line such as those described in WO 2018/098572. The resulting hybrid line may have approximately half the GaIT activity as the original homozygous line.

### REFERENCES

An, Y. Q., J. M. McDowell, S. Huang, E. C. McKinney, S. Chambliss et al., 1996 Strong, constitutive expression of the Arabidopsis ACT2/ACT8 actin subclass in vegetative tissues. Plant J 10: 107-121.
Bevan, M., 1984 Binary Agrobacterium vectors for plant transformation. Nucleic Acids Res 12: 8711-8721.
Diamos, A. G., and H. S. Mason, 2018 Chimeric 3′ flanking regions strongly enhance gene expression in plants. Plant Biotechnol J.
Duarte, M., and H. Laude, 1994 Sequence of the spike protein of the porcine epidemic diarrhoea virus. J Gen Virol 75 (Pt 5): 1195-1200.
Garabagi, F., E. Gilbert, A. Loos, M. D. McLean and J. C. Hall, 2012a Utility of the P19 suppressor of gene-silencing protein for production of therapeutic antibodies in Nicotiana expression hosts. Plant Biotechnology Journal 10: 1118-1128.
Garabagi, F., M. D. McLean and J. C. Hall, 2012b Transient and stable expression of antibodies in Nicotiana species. Methods in Molecular Biology 907: 389-408.
Gleba, Y., V. Klimyuk and S. Marillonnet, 2005 Magnifection—a new platform for expressing recombinant vaccines in plants. Vaccine 23: 2042-2048.
Grohs, B. M., Y. Niu, L. J. Veldhuis, S. Trabelsi, F. Garabagi et al., 2010 Plant-produced trastuzumab inhibits the growth of HER2 positive cancer cells. Journal of Agricultural and Food Chemistry 58: 10056-10063.
Hood, E. E., S. B. Gelvin, L. S. Melchers and A. Hoekema, 1993 New Agrobacterium helper plasmids for gene transfer to plants. Transgenic Research 2: 208-218.
Huang, Z., W. Phoolcharoen, H. Lai, K. Piensook, G. Cardineau et al., 2010 High-level rapid production of full-size monoclonal antibodies in plants by a single-vector DNA replicon system. Biotechnol Bioeng 106: 9-17.
Joshi, C. P., 1987 An inspection of the domain between putative TATA box and translation start site in 79 plant genes. Nucleic Acids Res 15: 6643-6653.
Kallolimath, S., C. Gruber, H. Steinkellner and A. Castilho, 2017 Promoter choice impacts the efficiency of plant glyco-engineering. Biotechnol J.
McLean, M. D., 2017 Trastuzumab Made in Plants Using vivoXPRESS® Platform Technology. J Drug Des Res 4(5): 1052. 4: 1052-1055.
McLean, M. D., and J. C. Hall, 2012 Biologics and biologic products. Ontario Society for Medical Technologists Advocate 19: 5-6.
Mor, T. S., Y. S. Moon, K. E. Palmer and H. S. Mason, 2003 Geminivirus vectors for high-level expression of foreign proteins in plant cells. Biotechnol Bioeng 81: 430-437.
Naim, F., K. Nakasugi, R. N. Crowhurst, E. Hilario, A. B. Zwart et al., 2012 Advanced engineering of lipid metabolism in Nicotiana benthamiana using a draft genome and the V2 viral silencing-suppressor protein. PLoS One 7: e52717.
Sainsbury, F., E. C. Thuenemann and G. P. Lomonossoff, 2009 pEAQ: versatile expression vectors for easy and quick transient expression of heterologous proteins in plants. Plant Biotechnol J 7: 682-693.
Strasser, R., A. Castilho, J. Stadlmann, R. Kunert, H. Quendler et al., 2009 Improved virus neutralization by plant-produced anti-HIV antibodies with a homogeneous beta1,4-galactosylated N-glycan profile. Journal of Biological Chemistry 284: 20479-20485.
Strasser, R., J. Stadlmann, M. Schahs, G. Stiegler, H. Quendler et al., 2008 Generation of glyco-engineered Nicotiana benthamiana for the production of monoclonal antibodies with a homogeneous human-like N-glycan structure. Plant Biotechnology Journal 6: 392-402.
Utiger, A., K. Tobler, A. Bridgen and M. Ackermann, 1995 Identification of the membrane protein of porcine epidemic diarrhea virus. Virus Genes 10: 137-148.
Nasab F P, Schultz B L, Gamarro F, Parodi A J, and Aebi M (2008). All in one: Leishmania major STT3 proteins substitute for the whole Oligosaccharyltransferase complex in Saccharomyces cerevisiae. Mol Biol Cell 19:3758-3768.
Zupan, J R, and P Zambryski. Transfer of T-DNA from Agrobacterium to the plant cell. Plant physiology vol. 107, 4 (1995): 1041-7.
Yadav, N S et al. Short direct repeats flank the T-DNA on a nopaline Ti plasmid. Proceedings of the National Academy of Sciences of the United States of America vol. 79, 20 (1982): 6322-6.
Slightom et al. (1986), Nucleotide sequence analysis of TL-DNA of Agrobacterium rhizogenes agropine type plasmid. Identification of open reading frames. The Journal of Biological Chemistry 261, 108-121

1. A plant T-DNA vector comprising a T-DNA region flanked by a Left Border sequence and a Right Border sequence, wherein the T-DNA region comprises a nucleic acid molecule encoding a protein of interest, optionally a post-translational modification (PTM) enzyme, and wherein the T-DNA region lacks a promoter sequence for the nucleic acid molecule.

2. The plant T-DNA vector of claim 1, wherein the T-DNA region lacks both a promoter sequence and a 5′ untranslated region (5′UTR) sequence for the nucleic acid molecule.

3. A plant T-DNA vector comprising a T-DNA region flanked by a Left Border sequence and/or a Right Border sequence, wherein the T-DNA region comprises a nucleic acid molecule encoding a protein of interest, optionally a post-translational modification (PTM) enzyme, and wherein
(a) the ATG start of the translation codon of the nucleic acid sequence encoding the protein of interest is directly adjacent to the Left Border sequence or the Right Border sequence;
(b) the ATG start of the translation codon of the nucleic acid sequence encoding the protein of interest is within 10, 9, 8, 7, 6, 5 or fewer nucleotides of the Left Border sequence or the Right Border sequence;
(c) the ATG start of the translation codon of the nucleic acid sequence encoding the protein of interest is directly adjacent to a UTR sequence, and the UTR sequence is directly adjacent to the Left Border sequence or the Right sequence region; or
(d) the ATG start of the translation codon of the nucleic acid sequence encoding the protein of interest is directly adjacent to a UTR sequence, and the UTR sequence is separated by an upstream sequence of 100 base pairs or less from the Left Border sequence or the Right Border sequence.

4. The plant T-DNA vector of claim 3, wherein the upstream sequence comprises a fragment of a promoter sequence.

5. The plant T-DNA vector of claim 4, wherein the fragment consists of no more than 3, 4, 5, 6, 7, 8, 9, 10, 15 or 20 contiguous base pairs of the promoter sequence.

6. The plant T-DNA vector of claim 3, wherein:
(a) the left border sequence comprises a sequence as set out in SEQ ID No: 23, or a sequence having at least 50, 60, 70, 75, 80, 85, 90, 95 or 99% sequence identity to SEQ ID No: 23.
(b) the right border sequence comprises SEQ ID No: 25, or a sequence having at least 50, 60, 70, 75, 80, 85, 90, 95 or 99% sequence identity to SEQ ID No: 25 and/or
(c) the UTR region comprises SEQ ID NO: 3, 5, 7 or 39, or a sequence having at least 50, 60, 70, 75, 80, 85, 90, 95 or 99% sequence identity to SEQ ID NO: SEQ ID NO: 3, 5, 7 or 39.

7. The plant T-DNA vector of claim 3, wherein the post-translational modification enzyme catalyzes the addition of oligosaccharide, galactose, fucose and/or sialic acid to a protein.

8. The plant T-DNA vector of claim 3, wherein the post-translational modification enzyme is GaIT, STT3D, FucT, a sialic acid synthesis enzyme or a transferase enzyme.

9. (canceled)

10. The plant T-DNA vector of claim 3, wherein the T-DNA region further comprises a second nucleic acid molecule encoding a recombinant protein.

11. The plant T-DNA vector of claim 10, wherein the recombinant protein is an antibody or fragment thereof, a therapeutic enzyme or a vaccine or a Virus Like Particle.

12-14. (canceled)

15. A kit comprising (a) the plant T-DNA vector of claim 1 and (b) a plant expression vector comprising a second nucleic acid molecule encoding a recombinant protein.

16. A genetically modified plant or plant cell comprising the plant T-DNA vector of claim 1.

17. The genetically modified plant or plant cell of claim 16, wherein the plant or plant cell further comprises a nucleic acid sequence encoding a recombinant protein.

18. The genetically modified plant or plant cell of claim 16, wherein the plant or plant cell is a tobacco plant or plant cell, optionally a Nicotiana plant or plant cell.

19. A method of obtaining a stable transgenic plant comprising (a) introducing the plant T-DNA vector of claim 1 into a plant or plant cell and (b) selecting a transgenic plant with a stable expression of the nucleic acid molecule.

20. A stable transgenic plant obtained by the method of claim 19.

21. The stable transgenic plant of claim 20, wherein the transgenic plant comprises a T-DNA insertion of the nucleic acid molecule at a single locus, optionally wherein the transgenic plant is homozygous for the T-DNA insertion.

22. The stable transgenic plant of claim 20, wherein the transgenic plant comprises a T-DNA insertion of the nucleic acid molecule at more than one locus.

23. A method of optimizing expression and/or glycosylation of a recombinant protein produced in a plant or plant cell, the method comprising:
(a) introducing into the plant or plant cell the plant T-DNA vector of claim 1,
(b) introducing a second nucleic acid molecule encoding the recombinant protein into the plant or plant cell, and
(c) growing the plant or plant cell to obtain a plant that expresses the recombinant protein.

24. A method of increasing the amount of galactosylation on a recombinant protein produced in a plant or plant cell, the method comprising:
(a) introducing into the plant or plant cell the plant T-DNA vector of claim 1,
(b) introducing a second nucleic acid molecule encoding the recombinant protein into the plant or plant cell, and
(c) growing the plant or plant cell to obtain a plant that expresses the recombinant protein,
and wherein the post-translational modification enzyme is GaIT.

25-33. (canceled)
